(19) United States
(12) Patent Application Publication
(10) Pub. No.: US 2021/0363245 A1
(43) Pub. Date: Nov. 25, 2021

(54) BICISTRONIC CHIMERIC ANTIGEN RECEPTORS TARGETING CD19 AND CD20 AND THEIR USES

(71) Applicant: The United States of America,as represented by the Secretary,Department of Health and Human Services, Bethesda, MD (US)

(72) Inventors: James N. Kochenderfer, Bethesda, MD (US); Shicheng Yang, Chapel Hill, NC (US)

(73) Assignee: The United States of America,as represented by the Secretary,Department of Health and Human Services, Bethesda, MD (US)

(21) Appl. No.: 17/276,959

(22) PCT Filed: Sep. 17, 2019
(86) PCT No.: PCT/US2019/051517

§ 371 Date: Mar. 17, 2021

Related U.S. Application Data
(60) Provisional application No. 62/732,263, filed on Sep. 17, 2018.

(51) Int. Cl. C07K 16/28; C07K 14/725; C07K 14/005; C07K 14/705; A61K 35/17; G01N 33/574; A61P 35/00

(52) U.S. Cl. CPC C07K 16/2803; C07K 14/7051; C07K 16/2887; C07K 14/005; C07K 14/70517; C07K 2317/622; A61K 35/17; G01N 33/57492; A61P 35/00; C07K 2319/03; C07K 2319/02; C07K 14/70521

(57) ABSTRACT

An embodiment of the invention provides nucleic acids comprising a nucleotide sequence encoding chimeric antigen receptor (CAR) amino acid constructs. Polypeptides, recombinant expression vectors, host cells, populations of cells, and pharmaceutical compositions relating to the CAR constructs are disclosed. Methods of detecting the presence of cancer in a mammal and methods of treating or preventing cancer in a mammal are also disclosed.

### CROSS-REFERENCE TO RELATED APPLICATION

This patent application claims the benefit of copending U.S. Provisional Patent Application No. 62/732,263, filed Sep. 17, 2018, which is incorporated by reference in its entirety herein.

### STATEMENT REGARDING FEDERALLY SPONSORED RESEARCH AND DEVELOPMENT

This invention was made with Government support under project number Z01 BC011417 by the National Institutes of Health, National Cancer Institute. The Government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: one 104,552 byte byte ASCII (text) file named “744443_ST25.txt” dated Sep. 13, 2019.

### BACKGROUND OF THE INVENTION

Cancer is a public health concern. Despite advances in treatments such as chemotherapy, the prognosis for many cancers, including hematological malignancies, may be poor. Accordingly, there exists an unmet need for additional treatments for cancer, particularly hematological malignancies.

### BRIEF SUMMARY OF THE INVENTION

An embodiment of the invention provides a nucleic acid comprising a nucleotide sequence encoding a chimeric antigen receptor (CAR) construct comprising: (a) a first CAR comprising a first antigen binding domain, a first transmembrane domain, and a first intracellular T cell signaling domain; (b) a second CAR comprising a second antigen binding domain, a second transmembrane domain, and a second intracellular T cell signaling domain; and (c) a cleavage sequence; wherein the cleavage sequence is positioned between the first and second CARs, wherein the first antigen binding domain of the first CAR has antigenic specificity for CD19, and wherein the second antigen binding domain of the second CAR has antigenic specificity for CD20.

Further embodiments of the invention provide related polypeptides encoded by the nucleic acids, recombinant expression vectors, host cells, populations of cells, and pharmaceutical compositions.

Additional embodiments of the invention provide related methods of detecting the presence of and treating or preventing cancer in a mammal.

### BRIEF DESCRIPTION OF THE DRAWINGS

FIGS. 1A-1J are schematics illustrating the structures of CARs. FIGS. 1A-1E illustrate bicistronic CARs. FIG. 1A illustrates that Hu1928-C2B8BB includes a leader sequence (SS) from human CD8α. After the SS is a scFv made up from N-terminus to C-terminus of Hu anti-CD19 scFv (including the heavy and light variable regions of Hu19 joined by a linker), human CD8α hinge and transmembrane domains, the intracellular T cell signaling domain of human CD28, the intracellular T cell signaling domain of human CD3ξ, a cleavage sequence that includes a F2A ribosomal skip and cleavage sequence (in this case, a foot-and-mouth disease virus [F2A] amino acid sequence), and the C2B8 anti-CD20 scFv (including the heavy and light variable regions of C2B8 joined by a linker). After the scFv, there are CD8α hinge and transmembrane domains followed by intracellular T cell signaling domain of human 4-1BB, followed by the intracellular T cell signaling domain CD3ξ. FIG. 1B illustrates that Hu1928-11B8BB has the same sequence as Hu1928-C2B8BB except the 11B8 light chain and heavy chain variable regions were substituted for the C2B8 light chain and heavy chain variable regions. FIG. 1C illustrates that Hu1928-8G6-5BB has the same sequence as Hu1928-C2B8BB except the 8G6 light chain and heavy chain variable regions were substituted for the C2B8 light chain and heavy chain variable regions. FIG. 1D illustrates that Hu1928-2.1.2BB has the same sequence as Hu1928-C2B8BB except the 2.1.2 light chain and heavy chain variable regions were substituted for the C2B8 light chain and heavy chain variable regions. FIG. 1E illustrates that Hu1928-GA101BB has the same sequence as Hu1928-C2B8BB except the GA101 light chain and heavy chain variable regions were substituted for the C2B8 light chain and heavy chain variable regions. FIGS. 1F-1J illustrate anti-CD20 CARs. FIG. 1F illustrates that C2B8-CD8BBZ includes a leader sequence (SS) from human CD8α. After the SS is a scFv made up from N-terminus to C-terminus of C2B8 (including the heavy and light variable regions of C2B8 joined by a linker), human CD8α hinge and transmembrane domains, CD8α hinge and transmembrane domains followed by intracellular T cell signaling domain of human 4-1BB, followed by the intracellular T cell signaling CD3ξ domain. FIG. 1G illustrates that 11B8-5CD8BBZ has the same sequence as C2B8-CD8BBZ except the 11B8 light chain and heavy chain variable regions were substituted for the C2B8 light chain and heavy chain variable regions. FIG. 1H illustrates that 8G6-5CD8BBZ has the same sequence as C2B8-CD8BBZ except the 8G6 light chain and heavy chain variable regions were substituted for the C2B8 light chain and heavy chain variable regions. FIG. 11 illustrates that 2.1.2-5CD8BBZ has the same sequence as C2B8-CD8BBZ except the 2.1.2 light chain and heavy chain variable regions were substituted for the C2B8 light chain and heavy chain variable regions. FIG. 1J illustrates that 1GA101-5CD8BBZ has the same sequence as C2B8-CD8BBZ except the GA101 light chain and heavy chain variable regions were substituted for the C2B8 light chain and heavy chain variable regions.

FIGS. 2A-2D are a set of plots showing T-cell expression of CAR Hu1928-C2B8BB (the CAR illustrated in FIG. 1A). Peripheral blood mononuclear cells were stimulated with the anti-CD3 monoclonal antibody OKT3. Two days later, the cells were transduced with gamma-retroviral vectors encoding the CARs Hu19-CD828Z (FIG. 2B), C2B8-CD828Z (FIG. 2C), Hu1928-C2B8BB (FIG. 2D). Nine days after transduction (day 11 of overall culture) the cells were stained with CD3 and an anti-CAR antibody. Plots were gated on live CD3+ lymphocytes. FIG. 2A is the plot from the untransduced control. FIGS. 2B and 2C are the plots from CARs Hu19-CD828Z (anti-CD19 CAR) and C2B8-CD828Z (anti-CD20 CAR), respectively.

FIG. 3 is a set of plots showing that the CAR-expressing CD8⁺ T cells degranulate in an antigen-specific manner. The T cells were left untransduced or were transduced with Hu19-CD828Z, C2B8-CD828Z, or Hu1928-C2B8BB. Eight days after transduction, the T cells were cultured for 4 hours with either the CD19⁺ target cells CD19-K562 or CD20⁺ target cells CD20-K562. Degranulation was measured by staining for CD107a. Plots were gated on live CD3⁺, CD8⁺ lymphocytes.

FIG. 4 is a set of plots showing that the CAR-expressing CD4⁺ T cells degranulate in an antigen-specific manner. The T cells were left untransduced or were transduced with Hu19-CD828Z, C2B8-CD828Z, or Hu1928-C2B8BB. Eight days after transduction, the T cells were cultured for 4 hours with either the CD19⁺ target cells CD19-K562 or CD20⁺ target cells CD20-K562. Degranulation was measured by staining for CD107a. Plots were gated on live CD3⁺, CD4⁺ lymphocytes.

FIG. 5 is a set of plots showing that the CAR T cells specifically recognize CD19 and/or CD20. Either CD8⁺ (top row) or CD4⁺ T cells (bottom row) expressing Hu1928-C2B8BB were co-cultured for 4 hours with the indicated target cells, and degranulation was assessed by staining for CD107a. The Hu1928-C2B8BB-expressing T cells degranulated to a greater degree when co-cultured with either CD19 or CD20-expressing target cells. Plots were gated on live, CD3⁺ lymphocytes and either CD8 (top row) or CD4 (bottom row).

FIG. 6 is a graph showing that Hu1928-C2B8BB-expressing T cells efficiently kill lymphoma cell line cells. The T cells were left untransduced (UT, open triangle pointing up) or were transduced with Hu19-CD828Z (open triangle pointing down), C2B8-CD828Z (open square), or Hu1928-C2B8BB (open circle). The T cells were co-cultured with cells of the CD19⁺, CD20⁺ lymphoma cell line Toledo (available from American Type Culture Collection [ATCC]) and with CCRF-CEM negative control cells that lack CD19 and CD20 expression. Cytotoxicity was determined as described in the examples.

FIGS. 7A-7D are a set of graphs showing Hu1828-C2B8-expressing T cells proliferate in response to CD19 and CD20. The T cells were transduced with Hu19-CD828Z, C2B8-CD828Z, or Hu1928-C2B8BB. Eleven days later, the CAR-expressing T cells were labeled with carboxyfluorescein diacetate succinimidyl ester (CFSE, Invitrogen) and cultured with irradiated CD19-K562 cells, CD20-K562 cells, or negative control NGFR-K562 cells (line with shading beneath). The co-cultures of T cells and irradiated target cells continued for 4 days, and then flow cytometry was performed on the cells to assess CFSE dilution as a measure of proliferation. The CAR-expressing T cells proliferated preferentially when exposed to cells expressing their target antigen (black lines). The cell counts on the y-axis also indicate that the number of T cells at the end of the 4-day culture period was higher when CAR T cells were exposed to target antigen(s). FIGS. 7A and 7B are graphs from cells that were transduced with Hu19-CD828Z, FIGS. 7C and 7D are graphs from cells that were transduced with Hu19-CD828Z, and FIGS. 7E and 7F are graphs from cells that were transduced with Hu1928-C2B8BB.

FIGS. 8A and 8B show CAR T-cell surface expression. Five days after transduction, expression of 4 different CARs was assessed (Hu19-CD828Z, C2B8-CD8BBZ, Hu1928-C2B8BB, and Hu1928-11B8BB). FIG. 8A shows staining with the anti-Hu19 antibody, which binds to the linker included in Hu19-CD828Z. Hu19-CD828Z bound to all T cells transduced with constructs including the Hu19-CD828Z CAR. FIG. 8B shows staining with an anti-rituximab antibody that binds to C2B8. The anti-rituximab antibody bound to the CAR constructs that contain C2B8. Plots were gated on live, CD3⁺ lymphocytes.

FIG. 9 shows that CD8⁺ CAR T cells degranulate in an antigen-specific manner. The T cells were transduced with either Hu19-CD828Z, C2B8-CD8BBZ, Hu1928-C2B8BB, or Hu1928-11B8BB. Five days later, the T cells were cultured for 4 hours with either CD19-K562 cells, CD20-K562 cells, or the negative control NGFR-K562 cells. Degranulation was assessed by CD107a degranulation. CD8⁺ T cells are shown. Plots were gated on live, CD8⁺, CD3+ lymphocytes.

FIG. 10 shows CD4⁺ CAR T cells degranulate in an antigen-specific manner. The T cells were transduced with either Hu19-CD828Z, C2B8-CD8BBZ, Hu1928-C2B8BB, or Hu1928-11B8BB. Five days later, these T cells were cultured for 4 hours with either CD19-K562 cells, CD20-K562 cells, or the negative control NGFR-K562 cells. Degranulation was assessed by CD107a degranulation. CD4⁺ T cells are shown. Plots were gated on live, CD4⁺, CD3⁺ lymphocytes.

FIGS. 11A-E show expression of anti-CD19 CARs in bicistronic constructs. The T cells were transduced with vectors encoding the indicated bicistronic CAR constructs, or left untransduced, and expression of the anti-CD19 CAR Hu19-CD828Z was evaluated with flow cytometry with the Kip-1 antibody. Plots were gated on live CD3⁺ lymphocytes. FIG. 11A shows the plot from cells that were untransduced. FIG. 11B shows the plot from the cells that were transduced with Hu1928-2.1.2BB. FIG. 11C shows the plot from the cells that were transduced with Hu1928-8G6-5BB. FIG. 11D shows the plot from the cells that were transduced with Hu1928-GA101BB. FIG. 11E shows the plot from the cells that were transduced with Hu1928-C2B8BB.

FIGS. 12A-E show expression of anti-CD20 CARs in bicistronic constructs. The T cells were transduced with vectors encoding the indicated bicistronic CAR constructs or left untransduced, and expression of the anti-CD20 CARs indicated by the second part of the CAR name after the hyphen was evaluated with flow cytometry with the Kip-4 antibody. Plots were gated on live CD3⁺ lymphocytes. FIG. 12A shows the plot from cells that were untransduced.

FIG. 12B shows the plot from when the expression of 2.1.2BB was evaluated. FIG. 12C shows the plot from when the expression of 8G6 was evaluated. FIG. 12D shows the plot from when GA101BB was evaluated. FIG. 12E shows the plot from when C2B8 was evaluated.

FIGS. 13A and 13B are a set of plots showing that CD4⁺ CAR T cells degranulate in a CD19-specific manner. The T cells transduced with the indicated bicistronic CAR constructs were cultured for 4 hours with either CD19-K562 cells or the negative control NGFR-K562 cells. Degranulation was assessed by CD107a degranulation. CD4⁺ T cells are shown. Plots were gated on live, CD4⁺, CD3⁺ lymphocytes. FIG. 13A shows the plots for (from left to right): (1) untransduced, NGFR-K562; (2) untransduced, CD19-K562; (3) Hu1928-2.1.2BB, NGFR-K562; (4) Hu1928-2.1.2BB, CD19-K562; (5) Hu1928-8G6-5BB, NGFR-K562; and (6) Hu1928-8G6-5BB, CD19-K562. FIG. 13B shows the plots for (from left to right): (1) Hu1928-GA101BB, NGFR-K562; (2) Hu1928-GA101BB, CD19-K562; (3) Hu1928-C2B8BB, NGFR-K562; and (4) Hu1928-C2B8BB, CD19-K562.

FIGS. 14A and 14B are a set of plots showing that CD4⁺ CAR T cells degranulate in a CD20-specific manner. The T cells transduced with the indicated bicistronic CAR constructs were cultured for 4 hours with either CD20-K562 cells or the negative control NGFR-K562 cells. Degranulation was assessed by CD107a degranulation. CD4⁺ T cells are shown. Plots were gated on live, CD4⁺, CD3⁺ lymphocytes. FIG. 14A shows the plots for (from left to right): (1) untransduced, NGFR-K562; (2) untransduced, CD20-K562; (3) Hu1928-2.1.2BB, NGFR-K562; (4) Hu1928-2.1.2BB, CD20-K562; (5) Hu1928-8G6-5BB, NGFR-K562; and (6) Hu1928-8G6-5BB, CD20-K562. FIG. 14B shows the plots for (from left to right): (1) Hu1928-GA101BB, NGFR-K562; (2) Hu1928-GA101BB, CD20-K562; (3) Hu1928-C2B8BB, NGFR-K562; and (4) Hu1928-C2B8BB, CD20-K562.

FIGS. 15A and 15B are a set of plots showing that CD8⁺ CAR T cells degranulate in a CD19-specific manner. The T cells transduced with the indicated bicistronic CAR constructs were cultured for 4 hours with either CD19-K562 cells or the negative control NGFR-K562 cells. Degranulation was assessed by CD107a degranulation. CD8⁺ T cells are shown. Plots were gated on live, CD8⁺, CD3⁺ lymphocytes. FIG. 15A shows the plots for (from left to right): (1) untransduced, NGFR-K562; (2) untransduced, CD19-K562; (3) Hu1928-2.1.2BB, NGFR-K562; (4) Hu1928-2.1.2BB, CD19-K562; (5) Hu1928-8G6-5BB, NGFR-K562; and (6) Hu1928-8G6-5BB, CD19-K562. FIG. 15B shows the plots for (from left to right): (1) Hu1928-GA101BB, NGFR-K562; (2) Hu1928-GA101BB, CD19-K562; (3) Hu1928-C2B8BB, NGFR-K562; and (4) Hu1928-C2B8BB, CD19-K562.

FIGS. 16A and 16B are a set of plots showing that CD8⁺ CAR T cells degranulate in a CD20-specific manner. The T cells transduced with the indicated bicistronic CAR constructs were cultured for 4 hours with either CD20-K562 cells or the negative control NGFR-K562 cells. Degranulation was assessed by CD107a degranulation. CD8⁺ T cells are shown. Plots were gated on live, CD8⁺, CD3⁺ lymphocytes. FIG. 16A shows the plots for (from left to right): (1) untransduced, NGFR-K562; (2) untransduced, CD20-K562; (3) Hu1928-2.1.2BB, NGFR-K562; (4) Hu1928-2.1.2BB, CD20-K562; (5) Hu1928-8G6-5BB, NGFR-K562; and (6) Hu1928-8G6-5BB, CD20-K562. FIG. 16B shows the plots for (from left to right): (1) Hu1928-GA101BB, NGFR-K562; (2) Hu1928-GA101BB, CD20-K562; (3) Hu1928-C2B8BB, NGFR-K562; and (4) Hu1928-C2B8BB, CD20-K562.

FIG. 17 is a graph showing that the constructs of the present invention can eradicate tumors in mice. The tumor volume in mm³ is shown on the y axis and the days after T cell infusion is on the x axis. The untransduced (open trianges) and SP6-CD828Z (open circles) transduced T cells allowed the tumor to increase in volume while the Hu1928-8G6-5BB (closed diamonds) and Hu1928-2.1.2BB (open squares) proved to be effective tumor treatments.

FIG. 18 is a graph showing that treatment with the CARs of the present invention can increase survival rate of mice. The percent survival is on the y axis and the days after T cell infursion is on the x axis. The mice treated with untransduced (open trianges) and SP6-CD828Z T (open circles) cells showed zero percent survival in less than 30 days while the Hu1928-8G6-5BB (closed diamonds) and Hu1928-2.1.2BB (open squares) proved to be effective tumor treatments with 100 percent survival after 50 days.

FIG. 19 is a schematic illustrating the generation of 2 separate CAR RNA molecules as it occurs in transduced T cells by the mechanism of ribosomal skipping caused by the presence of a 2A moiety according to an embodiment of the invention.

FIG. 20 is a set of plots showing expression of Hu19-CD828Z and Hu20-CD8BBZ on the surface of T cells five days after transduction with gamma-retroviruses encoding the Hu1928-2.1.2BB CAR construct. Gating was on CD4⁺ or CD8⁺ live, CD3⁺ lymphocytes. The CAR staining was performed with the Kip-1 antibody.

FIG. 21 is a set of plots showing the T cells from the same cultures shown in FIG. 20, but the CAR staining was performed with the Kip-4 antibody instead of the Kip-1 antibody.

FIG. 22 is a set of plots showing results of a representative CD107a assay after untransduced (UT) T cells, Hu1928-2.1.2BB T cells, Hu19-CD828Z T cells (Hu1928), and Hu20-CD8BBZ T cells (2.1.2BB) were cultured for 4 hours with target cells. The T cells degranulated specifically in response to target cells with Hu1928-2.1.2BB T cells degranulating in response to CD19⁺ and/or CD20⁺ target cells, Hu19-CD828Z T cells degranulating in response to CD19⁺ target cells, and Hu20-CD8BBZ degranulating in response to CD20⁺ target cells. Note that ST486 expresses low levels of CD19. FIG. 22 shows degranulation of CD8⁺ T cells.

FIG. 23 is a set of plots showing T cells from the same cultures shown in FIG. 22, but degranulation of CD4⁺ T cells instead of CD8⁺ T cells.

FIG. 24 is a set of graphs showing the results of a CFSE proliferation assay with T cells transduced with either Hu1928-2.1.2BB, Hu19-CD828Z, or Hu20-CD8BBZ. The area under the curves of the histograms is proportionate to the number of cells. The areas under the curves for NGFR-K562, either CD19-K562 (top row) or CD20-K562 (bottom row), and their overlaps, are as indicated in FIG. 24.

FIG. 25 is a graph showing the results of a cytotoxicity assay that compared survival of CD19⁺ and CD20⁺ Toledo human lymphoma cell line target cells relative to the survival of negative-control CCRF-CEM target cells that do not express CD19 or CD20.

FIG. 26 is a graph showing the results of a cytotoxicity assay that compared survival of T cells left untransduced or transduced with Hu1928-2.1.2BB or transduced with the negative-control CAR SP6-CD828Z. The human chronic lymphocytic leukemia cells were used as the CD19⁺ and CD20⁺ target cells.

FIG. 27 is a graph showing the tumor volume results of a dose-titration study. Four million ST486 cells were injected over 6 days to establish palpable intradermal tumors prior to CAR T cell infusion. Mice were treated with a single infusion of graded doses of Hu1928-2.1.2BB T cells as shown in FIG. 27.

FIG. 28 is a graph showing the survival rate results of the dose-titration experiment of FIG. 27.

FIG. 29 is a graph showing the tumor volume results of a study using a ST486 null (CD19−/−) cell line. Four million ST486 (CD19-/−) cells were injected over 6 days to establish palpable intradermal tumors prior to CAR T cell infusion. Mice were treated with a single infusion of of Hu1928-2.1.2BB T cells, Hu1928 T cells (Hu19-CD828Z), or 2.1.2BB T cells (2.1.2BB-CD8BBZ), as shown in FIG. 29.

FIG. 30 is a graph showing the survival rate results of the study of FIG. 29.

FIG. 31 is a graph showing the tumor volume results of a study using a NALM6 cell line (CD19⁺, CD20-negative). Four million NALM6 cells were injected intradermally into NSG to establish palpable intradermal tumors prior to CAR T cell infusion. Mice were left untreated or treated with a single infusion of Hu1928-2.1.2BB T cells, Hu1928 T cells, or 2.1.2BB T cells, as shown in FIG. 31.

FIG. 32 is a graph showing the survival rate results of the study of FIG. 31.

FIG. 33 is a graph showing the results of a study that measured the weight of mice used in a study. Solid tumors of ST486 cells were established in NSG mice and then the mice were infused with untransduced T cells or 5×10⁶ CAR⁺ T cells. The T cells expressed either Hu1928-2.1.2BB, Hu20-CD8BBZ, or Hu19-CD828Z.

FIG. 34 is a graph showing representative results from an immortalization study. The number of T cells transduced with MSGV1-Hu1928-2.1.2BB were observed in culture without exogenous interleukin-2 (IL-2). IL-2 was washed out of the culture on day 0.

### DETAILED DESCRIPTION OF THE INVENTION

An embodiment of the invention provides a nucleic acid comprising a nucleotide sequence encoding a chimeric antigen receptor (CAR) construct comprising: (a) a first CAR comprising a first antigen binding domain, a first transmembrane domain, and a first intracellular T cell signaling domain; (b) a second CAR comprising a second antigen binding domain, a second transmembrane domain, and a second intracellular T cell signaling domain; and (c) a cleavage sequence; wherein the cleavage sequence is positioned between the first and second CARs, wherein the first antigen binding domain of the first CAR has antigenic specificity for CD19, and wherein the second antigen binding domain of the second CAR has antigenic specificity for CD20.

A CAR is an artificially constructed hybrid protein or polypeptide containing an antigen binding domain of an antibody linked to T-cell signaling or T-cell activation domains. CARs have the ability to redirect T-cell specificity and reactivity toward a selected target in a non-MHC-restricted manner, exploiting the antigen-binding properties of monoclonal antibodies. The non-MHC-restricted antigen binding gives T-cells expressing CARs the ability to recognize an antigen independent of antigen processing, thus bypassing a major mechanism of tumor escape. Moreover, when expressed in T-cells, CARs advantageously do not dimerize with endogenous T-cell receptor (TCR) alpha and beta chains.

The first CAR has antigenic specificity for CD19 and the second CAR has antigenic specificity for CD20. The phrases “has antigenic specificity” and “elicit antigen-specific response,” as used herein, means that the CAR can specifically bind to and immunologically recognize an antigen, such that binding of the CAR to the antigen elicits an immune response.

CD19 (also known as B-lymphocyte antigen CD19, B4, and CVID3) is a cell surface molecule expressed only by B lymphocytes and follicular dendritic cells of the hematopoietic system. It is the earliest of the B-lineage-restricted antigens to be expressed and is present on most pre-B-cells and most non-T-cell acute lymphocytic leukemia cells and B-cell type chronic lymphocytic leukemia cells (Tedder and Isaacs, J. Immun., 143: 712-717 (1989)).

CD20 (also known as B-lymphocyte antigen CD20) is an activated-glycosylated phosphoprotein expressed on the surface of all B-cells. CD20 is found on B-cell lymphomas, hairy cell leukemia, B-cell chronic lymphocytic leukemia, transformed mycosis fungoides, and melanoma cancer stem cells.

The inventive bicistronic CAR constructs may provide any one or more of a variety of advantages. Although CAR T cells have been known to be a successful therapy, loss of CD19 expression after anti-CD19 CAR T-cell therapy has been found to be a mechanism for failure of this treatment approach (e.g., loss of CD19 expression has been detected in acute lymphoid leukemia and B-cell lymphomas). Further, some B-cell lymphoma cells lack CD19 expression. In some cases, CD19-negative malignancies retain CD20 expression. Loss of CD20 expression may also occur from malignant cells. The inventive bicistronic CAR constructs can target a malignancy that expresses CD19, CD20, or both CD19 and CD20. The inventive bicistronic CAR constructs may allow treatment of malignancies that lose expression of CD19 or CD20 if expression of one of the two antigens is retained. By targeting two antigens, CD19 and CD20, the inventive CAR constructs advantageously provide an alternative strategy for treating cancer.

Further, the inventive nucleic acids require only one gene therapy vector to engineer a patient's T cells to express two CARs: a first CAR that expresses CD19 and another CAR that expresses CD20. A single T cell can simultaneously express both CARs.

The first CAR comprises a first antigen binding domain. The first antigen binding domain recognizes and binds to CD19. The antigen binding domain of the CAR may comprise the antigen binding domain of an anti-CD19 antibody.

The second CAR comprises a second antigen binding domain. The second antigen binding domain recognizes and binds to CD20. The antigen binding domain of the CAR may comprise the antigen binding domain of an anti-CD20 antibody.

The first and second antigen binding domains may comprise any antigen binding portion of the anti-CD19 or anti-CD20 antibody, respectively. For example, the antigen binding domain may be a Fab fragment (Fab), F(ab′)2 fragment, diabody, triabody, tetrabody, single-chain variable region fragment (scFv), or a disulfide-stabilized variable region fragment (dsFv). In a preferred embodiment, the antigen binding domain is an scFv. An scFv is a truncated Fab fragment including the variable (V) domain of an antibody heavy chain linked to a V domain of an antibody light chain via a synthetic peptide, which can be generated using routine recombinant DNA technology techniques. The anti-CD19 or anti-CD20 antigen binding domains employed in the inventive CARs, however, are not limited to these exemplary types of antibody fragments.

The first antigen binding domain may comprise a light chain variable region and/or a heavy chain variable region of an anti-CD19 antibody. In an embodiment of the invention, the heavy chain variable region of the first antigen binding domain comprises a heavy chain complementarity determining region (CDR) 1, a heavy chain CDR2, and a heavy chain CDR3 of an anti-CD19 antibody. In an embodiment of the invention, the light chain variable region of the first antigen binding domain may comprise a light chain CDR1, a light chain CDR2, and a light chain CDR3 of an anti-CD19 antibody. In a preferred embodiment, the first antigen binding domain comprises all of a heavy chain CDR1, a heavy chain CDR2, a heavy chain CDR3, a light chain CDR1, a light chain CDR2, and a light chain CDR3 of an anti-CD19 antibody.

The second antigen binding domain may comprise a light chain variable region and/or a heavy chain variable region of an anti-CD20 antibody. In an embodiment of the invention, the heavy chain variable region of the second antigen binding domain comprises a heavy chain CDR1, a heavy chain CDR2, and a heavy chain CDR3 of an anti-CD20 antibody. In an embodiment of the invention, the light chain variable region of the second antigen binding domain may comprise a light chain CDR1, a light chain CDR2, and a light chain CDR3 of an anti-CD20 antibody. In a preferred embodiment, the second antigen binding domain comprises all of a light chain CDR1, a light chain CDR2, a light chain CDR3, a heavy chain CDR1, a heavy chain CDR2, and a heavy chain CDR3 of an anti-CD20 antibody.

In an embodiment of the invention, the first antigen binding domain of the CAR is the antigen binding domain of the scFv Hu19. The antigen binding domain of Hu19 specifically binds to CD19. The Hu19 scFv is described in Alabanza et al., Molecular Ther., 25: 2452-2465 (2017). The inventive first CAR may comprise all of the light chain CDR1, the light chain CDR2, the light chain CDR3, the heavy chain CDR1, the heavy chain CDR2, and the heavy chain CDR3 of Hu19.

In an embodiment of the invention, the second antigen binding domain of the CAR is the antigen binding domain of the antibody C2B8. The antigen binding domain of C2B8 specifically binds to CD20. The C2B8 antibody is described in U.S. Pat. No. 5,736,137, incorporated herein in its entirety. The inventive second CAR may comprise all of the light chain CDR1, the light chain CDR2, the light chain CDR3, the heavy chain CDR1, the heavy chain CDR2, and the heavy chain CDR3 of C2B8.

In an embodiment of the invention, the second antigen binding domain of the CAR is the antigen binding domain of the antibody 11B8. The antigen binding domain of 11B8 specifically binds to CD20. The 11B8 antibody is described in U.S. Patent Application 2004/0167319, incorporated herein in its entirety. The inventive second CAR may comprise all of the light chain CDR1, the light chain CDR2, the light chain CDR3, the heavy chain CDR1, the heavy chain CDR2, and the heavy chain CDR3 of 11B8.

In an embodiment of the invention, the second antigen binding domain of the CAR is the antigen binding domain of the antibody 8G6-5. The antigen binding domain of 8G6-5 specifically binds to CD20. The 8G6-5 antibody is described in U.S. Patent Application 2009/0035322, incorporated herein in its entirety. The inventive second CAR may comprise all of the light chain CDR1, the light chain CDR2, the light chain CDR3, the heavy chain CDR1, the heavy chain CDR2, and the heavy chain CDR3 of the antibody 8G6-5.

In an embodiment of the invention, the second antigen binding domain of the CAR is the antigen binding domain of the antibody 2.1.2. The antigen binding domain of 2.1.2 specifically binds to CD20. The 2.1.2 antibody is described in WO 2006/130458, incorporated herein in its entirety. The inventive second CAR may comprise all of the light chain CDR1, the light chain CDR2, the light chain CDR3, the heavy chain CDR1, the heavy chain CDR2, and the heavy chain CDR3 of the antibody 2.1.2.

In an embodiment of the invention, the second antigen binding domain of the CAR is the antigen binding domain of the antibody GA101. The antigen binding domain of GA101 specifically binds to CD20. The GA101 antibody is described in U.S. Pat. No. 9,539,251, incorporated herein in its entirety. The inventive second CAR may comprise all of the light chain CDR1, the light chain CDR2, the light chain CDR3, the heavy chain CDR1, the heavy chain CDR2, and the heavy chain CDR3 of the antibody GA101.

In an embodiment of the invention, the Hu19 antigen binding domain comprises a heavy chain variable region and a light chain variable region. The heavy chain variable region of the Hu19 antigen binding domain may comprise, consist of, or consist essentially of the amino acid sequence of SEQ ID NO: 6. The light chain variable region of the Hu19 antigen binding domain may comprise, consist of, or consist essentially of the amino acid sequence of SEQ ID NO: 4. Accordingly, in an embodiment of the invention, the Hu19 antigen binding domain comprises a heavy chain variable region comprising the amino acid sequence of SEQ ID NO: 6 and/or a light chain variable region comprising the amino acid sequence of SEQ ID NO: 4. Preferably, the Hu19 antigen binding domain comprises the amino acid sequences of both SEQ ID NOs: 6 and 4.

In an embodiment of the invention, the C2B8 antigen binding domain comprises a heavy chain variable region and a light chain variable region. The heavy chain variable region of the C2B8 antigen binding domain may comprise, consist of, or consist essentially of the amino acid sequence of SEQ ID NO: 18. The light chain variable region of the C2B8 antigen binding domain may comprise, consist of, or consist essentially of the amino acid sequence of SEQ ID NO: 17. Accordingly, in an embodiment of the invention, the C2B8 antigen binding domain comprises a heavy chain variable region comprising the amino acid sequence of SEQ ID NO: 18 and/or a light chain variable region comprising the amino acid sequence of SEQ ID NO: 17. Preferably, the C2B8 antigen binding domain comprises the amino acid sequences of both SEQ ID NOs: 17 and 18.

In an embodiment of the invention, the 11B8 antigen binding domain comprises a heavy chain variable region and a light chain variable region. The heavy chain variable region of the 11B8 antigen binding domain may comprise, consist of, or consist essentially of the amino acid sequence of SEQ ID NO: 13. The light chain variable region of the 11B8 antigen binding domain may comprise, consist of, or consist essentially of the amino acid sequence of SEQ ID NO: 12. Accordingly, in an embodiment of the invention, the 11B8 antigen binding domain comprises a heavy chain variable region comprising the amino acid sequence of SEQ ID NO: 13 and/or a light chain variable region comprising the amino acid sequence of SEQ ID NO: 12. Preferably, the 11B8 antigen binding domain comprises the amino acid sequences of both SEQ ID NOs: 12 and 13.

In an embodiment of the invention, the 8G6-5 antigen binding domain comprises a heavy chain variable region and a light chain variable region. The heavy chain variable region of the 8G6-5 antigen binding domain may comprise, consist of, or consist essentially of the amino acid sequence of SEQ ID NO: 26. The light chain variable region of the 8G6-5 antigen binding domain may comprise, consist of, or consist essentially of the amino acid sequence of SEQ ID NO: 25. Accordingly, in an embodiment of the invention, the 8G6-5 antigen binding domain comprises a heavy chain variable region comprising the amino acid sequence of SEQ ID NO: 26 and/or a light chain variable region comprising the amino acid sequence of SEQ ID NO: 25. Preferably, the 8G6-5 antigen binding domain comprises the amino acid sequences of both SEQ ID NOs: 25 and 26.

In an embodiment of the invention, the 2.1.2 antigen binding domain comprises a heavy chain variable region and a light chain variable region. The heavy chain variable region of the 2.1.2 antigen binding domain may comprise, consist of, or consist essentially of the amino acid sequence of SEQ ID NO: 22. The light chain variable region of the 2.1.2 antigen binding domain may comprise, consist of, or consist essentially of the amino acid sequence of SEQ ID NO: 21. Accordingly, in an embodiment of the invention, the 2.1.2 antigen binding domain comprises a heavy chain variable region comprising the amino acid sequence of SEQ ID NO: 22 and/or a light chain variable region comprising the amino acid sequence of SEQ ID NO: 21. Preferably, the 2.1.2 antigen binding domain comprises the amino acid sequences of both SEQ ID NOs: 21 and 22.

In an embodiment of the invention, the GA101 antigen binding domain comprises a heavy chain variable region and a light chain variable region. The heavy chain variable region of the GA101 antigen binding domain may comprise, consist of, or consist essentially of the amino acid sequence of SEQ ID NO: 30. The light chain variable region of the GA101 antigen binding domain may comprise, consist of, or consist essentially of the amino acid sequence of SEQ ID NO: 29. Accordingly, in an embodiment of the invention, the GA101 antigen binding domain comprises a heavy chain variable region comprising the amino acid sequence of SEQ ID NO: 30 and/or a light chain variable region comprising the amino acid sequence of SEQ ID NO: 29. Preferably, the GA101 antigen binding domain comprises the amino acid sequences of both SEQ ID NOs: 29 and 30.

The inventive second CAR may comprise a 11B8 antigen binding domain comprising one or more of a light chain CDR1 comprising, consisting of, or consisting essentially of the amino acid sequence of SEQ ID NO: 37; a light chain CDR2 comprising, consisting of, or consisting essentially of the amino acid sequence of SEQ ID NO: 38; and a light chain CDR3 comprising, consisting of, or consisting essentially of the amino acid sequence of SEQ ID NO: 39. Preferably, the 11B8 light chain comprises all of the amino acid sequences of SEQ ID NOs: 37-39.

The inventive second CAR may comprise a 11B8 antigen binding domain comprising one or more of a heavy chain CDR1 comprising, consisting of, or consisting essentially of the amino acid sequence of SEQ ID NO: 40; a heavy chain CDR2 comprising, consisting of, or consisting essentially of the amino acid sequence of SEQ ID NO: 41; and a heavy chain CDR3 comprising, consisting of, or consisting essentially of the amino acid sequence of SEQ ID NO: 42. Preferably, the 11B8 heavy chain comprises all of the amino acid sequences of SEQ ID NOs: 40-42.

In an embodiment, the 11B8 antigen binding domain comprises the amino acid sequences of all of SEQ ID NOs: 37-42.

The inventive second CAR may comprise a GA101 antigen binding domain comprising one or more of a light chain CDR1 comprising, consisting of, or consisting essentially of the amino acid sequence of SEQ ID NO: 43; a light chain CDR2 comprising, consisting of, or consisting essentially of the amino acid sequence of SEQ ID NO: 44; and a light chain CDR3 comprising, consisting of, or consisting essentially of the amino acid sequence of SEQ ID NO: 45. Preferably, the GA101 light chain comprises all of the amino acid sequences of SEQ ID NOs: 43-45.

The inventive second CAR may comprise a GA101 antigen binding domain comprising one or more of a heavy chain CDR1 comprising, consisting of, or consisting essentially of the amino acid sequence of SEQ ID NO: 46; a heavy chain CDR2 comprising, consisting of, or consisting essentially of the amino acid sequence of SEQ ID NO: 47; and a heavy chain CDR3 comprising, consisting of, or consisting essentially of the amino acid sequence of SEQ ID NO: 48. Preferably, the GA101 heavy chain comprises all of the amino acid sequences of SEQ ID NOs: 46-48.

In an embodiment, the GA101 antigen binding domain comprises all of the amino acid sequences of SEQ ID NOs: 43-48.

CDR sequences can be determined by one of skill in the art as a routine matter. Such methods and available resources are known in the art, for example see Wu, et al., J. Exp. Med., 132: 211-250 (1970), IMGT™, the international ImMunoGeneTics information system, and the freely available Paratome web server.

In an embodiment of the invention, the light chain variable region and the heavy chain variable region may be joined by an antigen binding domain linker peptide. The antigen binding domain linker peptide may be of any length and many comprise any amino acid sequence. For example, the antigen binding domain linker peptide may comprise or consist of any one or more of glycine, serine, lysine, proline, glutamic acid, and threonine, with or without other amino acid residues. In an embodiment of the invention, the antigen binding domain linker peptide may have a length of about 5 to about 100 amino acid residues, about 8 to about 75 amino acid residues, about 8 to about 50 amino acid residues, about 10 to about 25 amino acid residues, about 8 to about 30 amino acid residues, about 8 to about 40 amino acid residues, about 8 to about 50 amino acid residues, or about 12 to about 20 amino acid residues. In an embodiment of the invention, the antigen binding domain linker peptide has any of the foregoing lengths and consists of amino acid residues selected, independently, from the group consisting of glycine and serine. In an embodiment, the antigen binding domain linker peptide may comprise or consist of repeats of four glycines and one serine (G4S), for example, (G4S)³ (SEQ ID NO: 12). In an embodiment of the invention, the antigen binding domain linker peptide may comprise, consist, or consist essentially of, SEQ ID NO: 5 (GSTSGSGKPGSGEGSTKG). While the antigen binding domain may have a sequence from N-terminus to C-terminus of heavy-chain variable domain, linker, light-chain variable domain, in a preferred embodiment, the antigen binding domain has a sequence from N-terminus to C-terminus of light-chain variable domain, linker, heavy-chain variable domain.

In another embodiment, the each of the first and second CARs comprises a leader sequence (also referred to as a signal sequence). The leader sequence may be positioned at the amino terminus of one or both of the first and second antigen binding domains (e.g., one or both of the light chain variable region of the anti-CD19 antibody and the anti-CD20 antibody). The leader sequence may be a human leader sequence. The leader sequence may comprise any suitable amino acid sequence. In one embodiment, the leader sequence is a human granulocyte-macrophage colony-stimulating factor (GM-CSF) receptor leader sequence or a human CD8α leader sequence. For example, the antigen binding domain may comprise a human CD8α leader sequence comprising, consisting of, or consisting essentially of SEQ ID NO: 3. In an embodiment of the invention, while the leader sequence may facilitate expression of one or both of the first and second CARs on the surface of the cell, the presence of the leader sequence in one or both of the first and second expressed CARs may not be necessary in order for the CAR to function. In an embodiment of the invention, upon expression of one or both of the first and second CARs on the cell surface, all or a portion of the leader sequence may be cleaved off of the one or both of the first and second CARs. Accordingly, in an embodiment of the invention, the one or both of the first and second CARs lack a leader sequence.

In an embodiment of the invention, one or both of the first and second CARs comprise a hinge domain. One of ordinary skill in the art will appreciate that a hinge domain is a short sequence of amino acids that facilitates antibody flexibility (see, e.g., Woof et al., Nat. Rev. Immunol., 4(2): 89-99 (2004)). The hinge domain may be positioned between the antigen binding domain and the TM domain of one or both one or both of the first and second CARs. Preferably, the hinge domain is a human hinge domain. The hinge domain may comprise the hinge domain of human CD8α or human CD28. For example, the human hinge domain may comprise a sequence comprising, consisting of, or consisting essentially of the hinge domain of human CD8α.

The CAR may comprise a transmembrane (TM) domain. The TM domain can be any TM domain derived or obtained from any molecule known in the art. Preferably, the TM domain is a human TM domain. For example, the TM domain may comprise the TM domain of a human CD8α molecule or a human CD28 molecule. CD8 is a TM glycoprotein that serves as a co-receptor for the TCR, and is expressed primarily on the surface of cytotoxic T-cells. The most common form of CD8 exists as a dimer composed of a CD8α and CD8β chain. CD28 is expressed on T-cells and provides co-stimulatory signals for T-cell activation. CD28 is the receptor for CD80 (B7.1) and CD86 (B7.2). For example, the human TM domain may comprise a sequence comprising, consisting of, or consisting essentially of the TM domain of human CD8α.

The human CD8α hinge domain and human CD8α transmembrane domain may comprise, for example, a sequence comprising, consisting of, or consisting essentially of SEQ ID NO: 7.

One or both of the first and second CARs may comprise an intracellular (i.e., cytoplasmic) T-cell signaling domain. The intracellular T-cell signaling domain can be obtained or derived from a CD28 molecule, a CD3 zeta (ξ) molecule, an Fc receptor gamma (FcRγ) chain, a CD27 molecule, an OX40 molecule, a 4-1BB molecule, an inducible T-cell costimulatory protein (ICOS), or other intracellular signaling molecules known in the art, or modified versions of any of the foregoing. As discussed above, CD28 is a T-cell marker which is involved in T-cell co-stimulation. The intracellular T cell signaling domain of human CD28 may comprise, consist, or consist essentially of the amino acid sequence of SEQ ID NO: 8. CD3ξ associates with TCRs to produce a signal and contains immunoreceptor tyrosine-based activation motifs (ITAMs). The intracellular T cell signaling domain of human CD3ξ may comprise, consist, or consist essentially of the amino acid sequence of SEQ ID NO: 9. 4-1BB, also known as CD137, transmits a potent costimulatory signal to T-cells, promoting differentiation and enhancing long-term survival of T lymphocytes. The intracellular T cell signaling domain of human 4-1 in may comprise, consist, or consist essentially of the amino acid sequence of SEQ ID NO: 14. ICOS is a CD28-superfamily costimulatory molecule that is expressed on activated T cells. In a preferred embodiment, the CD28, CD3ξ, FcRγ, ICOS, 4-1BB, OX40, and CD27 are human.

One or both of the first and second CARs can comprise any one or more of the aforementioned TM domains and any one or more of the aforementioned intracellular T-cell signaling domains in any combination. For example, the inventive first CAR may comprise a CD8α hinge and TM domain and intracellular T-cell signaling domains of CD28 and CD3ξ. Alternatively, for example, the inventive second CAR may comprise a CD8α hinge and TM domain and intracellular T-cell signaling domains of 4-1BB and CD3ξ.

In one embodiment, the inventive CAR construct encodes, from the amino terminus to the carboxyl terminus, a CD8α leader sequence, an anti-CD19 scFv, human CD8α hinge and transmembrane domains, an intracellular T cell signaling domain of human CD28, an intracellular T cell signaling domain of the human CD3ξ molecule, a cleavage sequence, a CD8α leader sequence, an anti-CD20 scFv, human CD8α hinge and transmembrane domains, 4-1BB intracellular T cell signaling domain, and an intracellular T cell signaling domain of the human CD3ξ molecule.

The components of the bicistronic CAR constructs are set forth in Tables 1-5 below.

In one embodiment, the inventive first CAR comprises from the amino terminus to the carboxyl terminus, a leader sequence, an anti-CD19 scFv, human CD8α hinge and transmembrane domains, an intracellular T cell signaling domain of human CD28, and an intracellular T cell signaling domain of the human CD3ξ molecule.

In another embodiment, the inventive second CAR comprises from the amino terminus to the carboxyl terminus, a leader sequence, an anti-CD20 scFv, a human CD8α hinge and transmembrane domains, 4-1BB intracellular T cell signaling domain, and an intracellular T cell signaling domain of the human CD3ξ molecule.

Included in the scope of the invention are functional portions of the inventive CARs described herein. The term “functional portion” when used in reference to a CAR refers to any part or fragment of the CAR of the invention, which part or fragment retains the biological activity of the CAR of which it is a part (the parent CAR). Functional portions encompass, for example, those parts of a CAR that retain the ability to recognize target cells, or detect, treat, or prevent a disease, to a similar extent, the same extent, or to a higher extent, as the parent CAR. In reference to the parent CAR, the functional portion can comprise, for instance, about 10%, about 25%, about 30%, about 50%, about 60%, about 70%, about 80%, about 90%, about 95%, or more, of the parent CAR.

The functional portion can comprise additional amino acids at the amino or carboxy terminus of the portion, or at both termini, which additional amino acids are not found in the amino acid sequence of the parent CAR. Desirably, the additional amino acids do not interfere with the biological function of the functional portion, e.g., recognize target cells, detect cancer, treat or prevent cancer, etc. More desirably, the additional amino acids enhance the biological activity, as compared to the biological activity of the parent CAR.

Included in the scope of the invention are functional variants of the inventive CARs described herein. The term “functional variant” as used herein refers to a CAR, polypeptide, or protein having substantial or significant sequence identity or similarity to a parent CAR, which functional variant retains the biological activity of the CAR of which it is a variant. Functional variants encompass, for example, those variants of the CAR described herein (the parent CAR) that retain the ability to recognize target cells to a similar extent, the same extent, or to a higher extent, as the parent CAR. In reference to the parent CAR, the functional variant can, for instance, be at least about 30%, about 50%, about 75%, about 80%, about 90%, about 98% or more identical in amino acid sequence to the parent CAR.

A functional variant can, for example, comprise the amino acid sequence of the parent CAR with at least one conservative amino acid substitution. Alternatively or additionally, the functional variants can comprise the amino acid sequence of the parent CAR with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent CAR.

Amino acid substitutions of the inventive CARs are preferably conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gln, Ser, Thr, Tyr, etc.), an amino acid with a beta-branched side-chain substituted for another amino acid with a beta-branched side-chain (e.g., Ile, Thr, and Val), an amino acid with an aromatic side-chain substituted for another amino acid with an aromatic side chain (e.g., His, Phe, Trp, and Tyr), etc.

The CAR can consist essentially of the specified amino acid sequence or sequences described herein, such that other components, e.g., other amino acids, do not materially change the biological activity of the functional variant.

The CARs of embodiments of the invention (including functional portions and functional variants) can be of any length, i.e., can comprise any number of amino acids, provided that the CARs (or functional portions or functional variants thereof) retain their biological activity, e.g., the ability to specifically bind to antigen, detect diseased cells in a mammal, or treat or prevent disease in a mammal, etc. For example, the CAR can be about 50 to about 1000 amino acids long, such as 50, 70, 75, 100, 125, 150, 175, 200, 300, 400, 500, 600, 700, 800, 900, 1000 or more amino acids in length.

The CARs of embodiments of the invention (including functional portions and functional variants of the invention) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, 3-phenylserine p-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine.

The CARs of embodiments of the invention (including functional portions and functional variants) can be glycosylated, amidated, carboxylated, phosphorylated, esterified, N-acylated, cyclized via, e.g., a disulfide bridge, or converted into an acid addition salt and/or optionally dimerized or polymerized, or conjugated.

The CARs of embodiments of the invention (including functional portions and functional variants thereof) can be obtained by methods known in the art. The CARs may be made by any suitable method of making polypeptides or proteins. For example, CARs can be recombinantly produced using the nucleic acids described herein using standard recombinant methods. See, for instance, Green and Sambrook, Molecular Cloning: A Laboratory Manual, 4^{th} ed., Cold Spring Harbor Press, Cold Spring Harbor, N.Y. (2012). Alternatively, the CARs described herein (including functional portions and functional variants thereof) can be commercially synthesized by companies, such as Synpep (Dublin, Calif.), Peptide Technologies Corp. (Gaithersburg, Md.), and Multiple Peptide Systems (San Diego, Calif.). In this respect, the inventive CARs can be synthetic, recombinant, isolated, and/or purified.

Further provided by an embodiment of the invention is a nucleic acid comprising a nucleotide sequence encoding any of the CARs described herein (including functional portions and functional variants thereof). The nucleic acids of the invention may comprise a nucleotide sequence encoding any of the leader domains, hinge domains, antigen binding domains, cleavage sequences, TM domains, and intracellular T cell signaling domains described herein. Accordingly, an embodiment of the invention provides a nucleic acid comprising a nucleic acid comprising a nucleotide sequence encoding CAR construct comprising (a) a first CAR comprising a first antigen binding domain, a first transmembrane domain, and a first intracellular T cell signaling domain, (b) a second CAR comprising a second antigen binding domain, a second transmembrane domain, and a second intracellular T cell signaling domain, and (c) a cleavage sequence, wherein the cleavage sequence is positioned between the first and second CARs, wherein the first antigen binding domain of the first CAR has antigenic specificity for CD19, and wherein the second antigen binding domain of the second CAR has antigenic specificity for CD20.

In embodiments of the invention, the first and/or second CAR may be provided in combination with a regulatory element capable of modulating the anti-CD19 and/or anti-CD19 activity of a host cell expressing the CAR. The regulatory element may regulate the anti-CD19 and/or anti-CD20 activity of a host cell expressing the CAR. Accordingly, an embodiment of the invention provides a system comprising: (a) a nucleotide sequence encoding a first CAR, wherein the first CAR comprises a first antigen binding domain, a TM domain, and an intracellular T cell signaling domain, and wherein the first CAR has antigenic specificity for CD19; (b) a nucleotide sequence encoding a second CAR, wherein the second CAR comprises a second antigen binding domain, a TM domain, and an intracellular T cell signaling domain, and wherein the second CAR has antigenic specificity for CD20; (c) a cleavage sequence, and (d) a regulatory element capable of modulating the anti-CD19 and/or anti-CD20 activity of a host cell expressing the CAR. The regulatory element may regulate the anti-CD19 and/or anti-CD20 activity of a host cell expressing the first and/or second CAR. For example, the regulatory element may act as an “on” or “off” switch.

In an embodiment of the invention, the regulatory element downregulates the anti-CD19 and/or anti-CD20 activity of the host cell expressing the first and/or second CAR. For example, the regulatory element kills the host cell expressing the first and/or second CAR. In this regard, the regulatory element is a suicide gene. In an embodiment of the invention, the regulatory element is an inducible dimerization kill switch. An example of an inducible dimerization kill switch is the IC9 suicide gene. Another example of an inducible dimerization kill switch is an element which provides for small-molecule-induced dimerization of the intracellular signaling domain of Fas, which induces apoptosis via a caspase-8-dependent pathway. This approach may be used to induce apoptosis using a small molecule made by fusing two molecules of the drug calcineurin (Spencer et al., Curr. Biol., 6: 839-47 (1996); Belshaw et al., Chem. Biol., 3: 731-38 (1996)) or the FKBP/AP1903 dimerizer system described herein (Thomis et al., Blood, 97: 1249-57 (2001)).

In an embodiment of the invention, the regulatory element is a cell surface marker. The cell surface marker may be co-expressed with the first and/or second CAR. Administration of an antibody targeting the cell surface marker may reduce or eliminate the first and/or second CAR-expressing host cells. Such cell surface markers may be useful as a safety mechanism to deplete CAR-positive cells in vivo. In vivo depletion may occur by one or both of complement-mediated lysis of opsonized cells and antibody-mediated cell-dependent cytotoxicity. For example, cells transduced with a cell surface marker which is a CD8α stalk with two rituximab (anti-CD20) mimotopes can be depleted with rituximab (Philip et al., Blood, 124: 1277-87 (2014)). Other examples of cell surface markers which may be targeted for depletion by an antibody include CD20 (Griffioen et al., Haematologica, 94: 1316-20 (2009)), c-myc epitope tag (Kieback et al., PNAS, 105: 623-28 (2008)), and truncated versions of the human epidermal growth factor receptor. The truncated epidermal growth factor receptor may lack one or both of the ligand-binding and intracellular signaling domains but retain the epitope for cetuximab binding (Wang et al., Blood, 118: 1255-63 (2011)).

The regulatory element may be an inhibitory receptor. For example, antigen-specific inhibitory chimeric antigen receptors (iCARs) may preemptively constrain T cell responses. Such iCARs may selectively limit cytokine secretion, cytotoxicity, and proliferation induced through the endogenous T cell receptor or an activating chimeric receptor (Fedorov et al., Sci. Transl. Med., 5:215ra172 (2013)).

In an embodiment of the invention, the regulatory element upregulates the anti-CD19 and/or anti-CD20 activity of the host cell. In this regard, the regulatory element may act as an “on” switch to control expression or activity of the first and/or second CAR to occur where and when it is needed.

For example, the regulatory element may be an element which confers dependence on small-molecule ligands for cell survival or activity. An example of such an element may be a drug-responsive, ribozyme-based regulatory device linked to growth cytokine targets to control cell (e.g., T cell) proliferation (Chen et al., PNAS, 107(19): 8531-6 (2010)). Another example may be to design the antigen-binding and intracellular signaling components of the CAR to assemble only in the presence of a heterodimerizing small molecule (Wu et al., Science, 350(6258):aab4077 (2015)).

Other potential regulatory elements may include elements which control the location of transgene integration (Schumann et al., PNAS, 112(33): 10437-42 (2015)) or a genetic deletion which produces an auxotrophic cell (e.g., T cell).

In another embodiment of the invention, the nucleotide sequence encoding the first and/or second CAR is RNA. Introducing CAR mRNA into cells may result in transient expression of the CAR. With this approach, the mRNA may persist for a few days, but there may be an antitumor effect with minimal on-target toxicity (Beatty et al., Cancer Immunol. Res., 2(2): 112-20 (2014)).

In an embodiment of the invention, the first and/or second CAR is provided in combination with a suicide gene. The product of the suicide gene may, advantageously, provide on-demand reduction or elimination of anti-CD19 and/or anti-CD20 activity CAR-expressing cells.

As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine daminase, inducible caspase 9 (IC9) gene, purine nucleoside phosphorylase, and nitroreductase.

The suicide gene may be the IC9 gene. The product of the IC9 gene contains part of the proapoptotic protein human caspase 9 (“caspase 9 component”) fused to a binding domain derived from human FK-506 binding protein (FKBP12 component). Activation of the caspase 9 domain of IC9 is dependent on dimerization of IC9 proteins that occurs when a small molecule drug, rimiducid (AP1903), binds to the FKBP12 moiety of IC9. After caspase 9 is activated, the cells carrying the IC9 gene undergo apoptosis.

In an embodiment of the invention, the nucleic acid comprises a nucleotide sequence encoding a cleavage sequence that is positioned between the first and second CARs. In an embodiment of the invention, the cleavage sequence is cleavable. In this regard, the amino acid sequence encoded by the inventive nucleic acids may be cleaved such that two proteins are produced: a first protein encoded by the nucleotide sequence encoding the first CAR and a second protein encoded by the nucleotide sequence encoding the second CAR.

In an embodiment, the cleavable cleavage sequence comprises a “self cleaving” sequence. In an embodiment, the “self cleaving” sequence is a “self cleaving” 2A peptide. “Self cleaving” 2A peptides are described, for example, in Liu et al., Sci. Rep., 7(1): 2193 (2017), and Szymczak et al., Nature Biotechnol., 22(5): 589-594 (2004). 2A peptides are viral oligopeptides that mediate cleavage of polypeptides during translation in eukaryotic cells. The designation “2A” refers to a specific region of the viral genome. Without being bound to a particular theory or mechanism, it is believed that the mechanism of 2A-mediated “self cleavage” is ribosome skipping of the formation of a glycyl-prolyl peptide bond at the C-terminus of the 2A peptide. Different 2A peptides may comprise, at the C-terminus, the consensus amino acid sequence of GDVEX₁NPGP (SEQ ID NO: 49), wherein X₁ of SEQ ID NO: 49 is any naturally occurring amino acid residue. In an embodiment of the invention, the cleavable ribosomal skip sequence is a porcine teschovirus-1 2A (P2A) amino acid sequence, equine rhinitis A virus (E2A) amino acid sequence, thosea asigna virus 2A (T2A) amino acid sequence, or foot-and-mouth disease virus (F2A) amino acid sequence. In an embodiment of the invention, the ribosomal skip sequence is a 2A peptide amino acid sequence comprising, consisting, or consisting essentially of, the amino acid sequence of (F2A).

In an embodiment, the cleavable cleavage sequence comprises an enzyme-cleavable sequence. In an embodiment, the enzyme-cleavable sequence is a furin-cleavable sequence. Exemplary furin-cleavable sequences are described in Duckert et al., Protein Engineering, Design & Selection, 17(1): 107-112 (2004) and U.S. Pat. No. 8,871,906, each of which is incorporated herein by reference. In an embodiment of the invention, the furin-cleavable sequence is represented by the formula P4-P3-P2-P1 (Formula I), wherein P4 is an amino acid residue at the amino end, P1 is an amino acid residue at the carboxyl end, P1 is an arginine or a lysine residue, and the sequence is cleavable at the carboxyl end of P1 by furin. In another embodiment of the invention, the furin-cleavable sequence of Formula I (i) further comprises amino acid residues represented by P6-P5 at the amino end, (ii) further comprises amino acid residues represented by P1′-P2′ at the carboxyl end, (iii) wherein if P1 is an arginine or a lysine residue, P2′ is tryptophan, and P4 is arginine, valine or lysine, provided that if P4 is not arginine, then P6 and P2 are basic residues, and (iv) the sequence is cleavable at the carboxyl end of P1 by furin. In an embodiment of the invention, the furin-cleavable sequence comprises R-X₁-X₂-R, wherein X₁ is any naturally occurring amino acid and X₂ is arginine or lysine.

In an embodiment of the invention, the cleavage sequence comprises an enzyme-cleavable sequence and any “self cleaving” sequence. In an embodiment of the invention, the cleavage sequence comprises an enzyme-cleavable sequence (e.g., a furin cleavable sequence), a spacer (e.g., SGSG [SEQ ID NO: 50]), and a “self cleaving” sequence (e.g., F2A). In an embodiment of the invention, the cleavage sequence is an amino acid sequence comprising, consisting, or consisting essentially of, the amino acid sequence of (SEQ ID NO: 10).

In an embodiment, the nucleic acid sequence may comprise, consist of, or consist essentially of the nucleotide sequence of any one of SEQ ID NO: 1 (Hu1928-11B8BB), SEQ ID NO: 15 (Hu1928-C2B8BB), SEQ ID NO: 19 (Hu1928-2.1.2BB), SEQ ID NO: 23 (Hu1928-8G6BB), or SEQ ID NO: 27 (Hu1928-GA101BB).

In an embodiment, the nucleic acid sequence may encode a sequence that comprises, consists of, or consists essentially of SEQ ID NO: 2 (Hu1928-11B8BB), SEQ ID NO: 16 (Hu1928-C2B8BB), SEQ ID NO: 20 (Hu1928-2.1.2BB), SEQ ID NO: 24 (Hu1928-8G6BB), or SEQ ID NO: 28 (Hu1928-GA101BB). Another embodiment of the invention provides a nucleic acid comprising a nucleotide sequence encoding an anti-CD19 CAR comprising an antigen binding domain, a TM domain, and an intracellular T cell signaling domain, wherein the antigen binding domain has antigenic specificity for CD19. The anti-CD19 CAR may be as described herein with respect to other aspects of the invention.

Another embodiment of the invention provides a nucleic acid comprising a nucleotide sequence encoding an anti-CD20 CAR comprising an antigen binding domain, a TM domain, and an intracellular T cell signaling domain, wherein the antigen binding domain has antigenic specificity for CD20. The anti-CD20 CAR may be as described herein with respect to other aspects of the invention.

A further embodiment of the invention provides a nucleic acid, wherein the CAR construct comprises exactly two CARs being the first and second CARs, respectively.

“Nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. In some embodiments, the nucleic acid does not comprise any insertions, deletions, inversions, and/or substitutions. However, it may be suitable in some instances, as discussed herein, for the nucleic acid to comprise one or more insertions, deletions, inversions, and/or substitutions.

The nucleic acids of an embodiment of the invention may be recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

A recombinant nucleic acid may be one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques, such as those described in Green and Sambrook, supra. The nucleic acids can be constructed based on chemical synthesis and/or enzymatic ligation reactions using procedures known in the art. See, for example, Green and Sambrook, supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, Colo.) and Synthegen (Houston, Tex.).

The nucleic acid can comprise any isolated or purified nucleotide sequence which encodes any of the CARs or functional portions or functional variants thereof. Alternatively, the nucleotide sequence can comprise a nucleotide sequence which is degenerate to any of the sequences or a combination of degenerate sequences.

An embodiment of the invention also provides an isolated or purified nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions may hybridize under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the inventive CARs (alone or in combination with a suicide gene). It is generally appreciated that conditions can be rendered more stringent by the addition of increasing amounts of formamide.

The invention also provides a nucleic acid comprising a nucleotide sequence that is at least about 70% or more, e.g., about 80%, about 90%, about 91%, about 92%, about 93%, about 94%, about 95%, about 96%, about 97%, about 98%, or about 99% identical to any of the nucleic acids described herein.

In an embodiment, the nucleic acids of the invention can be incorporated into a recombinant expression vector. In this regard, an embodiment of the invention provides recombinant expression vectors comprising any of the nucleic acids of the invention. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the invention are not naturally-occurring as a whole. However, parts of the vectors can be naturally-occurring. The inventive recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. Preferably, the non-naturally occurring or altered nucleotides or internucleotide linkages do not hinder the transcription or replication of the vector.

In an embodiment, the recombinant expression vector of the invention can be any suitable recombinant expression vector, and can be used to transform or transfect any suitable host cell. Suitable vectors include those designed for propagation and expansion or for expression or both, such as plasmids and viruses. The vector can be selected from the group consisting of the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λZapII (Stratagene), λEMBL4, and λNM1149, also can be used. Examples of plant expression vectors include pBI01, pBI101.2, pBI101.3, pBI121 and pBIN19 (Clontech). Examples of animal expression vectors include pEUK-Cl, pMAM, and pMAMneo (Clontech). The recombinant expression vector may be a viral vector, e.g., a retroviral vector (e.g., a gamma-retroviral vector) or a lentiviral vector.

In an embodiment, the recombinant expression vectors of the invention can be prepared using standard recombinant DNA techniques described in, for example, Sambrook and Green, supra. Constructs of expression vectors, which are circular or linear, can be prepared to contain a replication system functional in a prokaryotic or eukaryotic host cell. Replication systems can be derived, e.g., from ColEl, 2μ plasmid, λ, SV40, bovine papilloma virus, and the like.

The recombinant expression vector may comprise regulatory sequences, such as transcription and translation initiation and termination codons, which are specific to the type of host cell (e.g., bacterium, fungus, plant, or animal) into which the vector is to be introduced, as appropriate, and taking into consideration whether the vector is DNA- or RNA-based. The recombinant expression vector may comprise restriction sites to facilitate cloning. In addition to the inventive nucleic acid sequence encoding the CARs (alone or in combination with a suicide gene), the recombinant expression vector preferably comprises expression control sequences, such as promoters, enhancers, polyadenylation signals, transcription terminators, internal ribosome entry sites (IRES), and the like, that provide for the expression of the nucleic acid sequence in a host cell.

The recombinant expression vector can include one or more marker genes, which allow for selection of transformed or transfected host cells. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or nonnative promoter operably linked to the nucleotide sequence encoding the CARs (including functional portions and functional variants thereof) (alone or in combination with a suicide gene), or to the nucleotide sequence which is complementary to or which hybridizes to the nucleotide sequence encoding the CARs (alone or in combination with a suicide gene). The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, or a promoter found in the long-terminal repeat of the murine stem cell virus.

The inventive recombinant expression vectors can be designed for either transient expression, for stable expression, or for both. Also, the recombinant expression vectors can be made for constitutive expression or for inducible expression.

An embodiment of the invention further provides a host cell comprising any of the recombinant expression vectors described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5a E. coli cells, Chinese hamster ovarian cells, monkey VERO cells, COS cells, HEK293 cells, and the like. For purposes of amplifying or replicating the recombinant expression vector, the host cell may be a prokaryotic cell, e.g., a DH5c cell. For purposes of producing a recombinant CAR, the host cell may be a mammalian cell. The host cell may be a human cell. The host cell can be of any cell type, can originate from any type of tissue, and can be of any developmental stage. The host cell may be a peripheral blood lymphocyte (PBL) or a peripheral blood mononuclear cell (PBMC).

In an embodiment of the invention, the host cell is a T cell. For purposes herein, the T cell can be any T cell, such as a cultured T cell, e.g., a primary T cell, or a T cell from a cultured T cell line, e.g., Jurkat, SupT1, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. The T cell may be a human T cell. The T cell may be a T cell isolated from a human. The T cell can be any type of T cell and can be of any developmental stage, including but not limited to, CD4⁺/CD8⁺ double positive T cells, CD4⁺ helper T cells, e.g., Th₁ and Th₂ cells, CD8⁺ T cells (e.g., cytotoxic T cells), tumor infiltrating cells, memory T cells, naïve T cells, and the like. The T cell may be a CD8⁺ T cell or a CD4⁺ T cell.

In an embodiment of the invention, the host cell is a natural killer (NK) cell. NK cells are a type of cytotoxic lymphocyte that plays a role in the innate immune system. NK cells are defined as large granular lymphocytes and constitute the third kind of cells differentiated from the common lymphoid progenitor which also gives rise to B and T lymphocytes (see, e.g., Immunobiology, 9^{th} ed., Janeway et al., eds., Garland Publishing, New York, N.Y. (2016)). NK cells differentiate and mature in the bone marrow, lymph node, spleen, tonsils, and thymus. Following maturation, NK cells enter into the circulation as large lymphocytes with distinctive cytotoxic granules. NK cells are able to recognize and kill some abnormal cells, such as, for example, some tumor cells and virus-infected cells, and are thought to be important in the innate immune defense against intracellular pathogens. As described above with respect to T-cells, the NK cell can be any NK cell, such as a cultured NK cell, e.g., a primary NK cell, or an NK cell from a cultured NK cell line, or an NK cell obtained from a mammal. If obtained from a mammal, the NK cell can be obtained from numerous sources, including but not limited to blood, bone marrow, lymph node, the thymus, or other tissues or fluids. NK cells can also be enriched for or purified. The NK cell preferably is a human NK cell (e.g., isolated from a human). NK cell lines are available from, e.g., the American Type Culture Collection (ATCC, Manassas, Va.) and include, for example, NK-92 cells (ATCC CRL-2407), NK92MI cells (ATCC CRL-2408), and derivatives thereof.

Also provided by an embodiment of the invention is a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cell, a muscle cell, a brain cell, etc. Alternatively, the population of cells can be a substantially homogeneous population, in which the population comprises mainly host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment of the invention, the population of cells is a clonal population comprising host cells comprising a recombinant expression vector as described herein.

The inventive recombinant expression vectors encoding the CARs may be introduced into a cell by “transfection,” “transformation,” or “transduction.” “Transfection,” “transformation,” or “transduction,” as used herein, refer to the introduction of one or more exogenous polynucleotides into a host cell by using physical or chemical methods. Many transfection techniques are known in the art and include, for example, calcium phosphate DNA co-precipitation; DEAE-dextran; electroporation; cationic liposome-mediated transfection; tungsten particle-facilitated microparticle bombardment; and strontium phosphate DNA co-precipitation. Phage or viral vectors can be introduced into host cells, after growth of infectious particles in suitable packaging cells, many of which are commercially available.

Included in the scope of the invention are conjugates, e.g., bioconjugates, comprising any of the inventive CARs (including any of the functional portions or variants thereof), nucleic acids, recombinant expression vectors, host cells, or populations of host cells. Conjugates, as well as methods of synthesizing conjugates in general, are known in the art.

CARs (including functional portions and variants thereof) (alone or in combination with a suicide gene product), nucleic acids, systems, protein(s) and combination(s) of proteins encoded by the nucleic acids, recombinant expression vectors, and host cells (including populations thereof), all of which are collectively referred to as “inventive CAR materials” hereinafter, can be isolated and/or purified. The term “isolated” as used herein means having been removed from its natural environment. The term “purified” or “isolated” does not require absolute purity or isolation; rather, it is intended as a relative term. Thus, for example, a purified (or isolated) host cell preparation is one in which the host cell is more pure than cells in their natural environment within the body. Such host cells may be produced, for example, by standard purification techniques. In some embodiments, a preparation of a host cell is purified such that the host cell represents at least about 50%, for example at least about 70%, of the total cell content of the preparation. For example, the purity can be at least about 50%, can be greater than about 60%, about 70% or about 80%, or can be about 100%.

The inventive CAR materials can be formulated into a composition, such as a pharmaceutical composition. In this regard, an embodiment of the invention provides a pharmaceutical composition comprising any of the inventive CAR materials and a pharmaceutically acceptable carrier. The inventive pharmaceutical compositions containing any of the inventive CAR materials can comprise more than one inventive CAR material, e.g., a CAR and a nucleic acid, or two or more different CARs. Alternatively, the pharmaceutical composition can comprise an inventive CAR material in combination with other pharmaceutically active agents or drugs, such as chemotherapeutic agents, e.g., asparaginase, busulfan, carboplatin, cisplatin, cyclophosphamide, daunorubicin, doxorubicin, fludarabine, fluorouracil, gemcitabine, hydroxyurea, methotrexate, paclitaxel, rituximab, vinblastine, vincristine, etc. In a preferred embodiment, the pharmaceutical composition comprises the inventive host cell or populations thereof.

Preferably, the carrier is a pharmaceutically acceptable carrier. With respect to pharmaceutical compositions, the carrier can be any of those conventionally used for the particular inventive CAR material under consideration. Such pharmaceutically acceptable carriers are well-known to those skilled in the art and are readily available to the public. It is preferred that the pharmaceutically acceptable carrier be one which has no detrimental side effects or toxicity under the conditions of use.

The choice of carrier will be determined in part by the particular inventive CAR material, as well as by the particular method used to administer the inventive CAR material. In a preferred embodiment, the CARs are expressed by a host cell, which is preferably a T cell or an NK cell, and host cells expressing the CARs are administered to a patient. These cells could be autologous or allogeneic in relation to the recipient of the cells. A nucleic acid encoding the CARs may be introduced to the cells by any of a variety of methods of genetic modification including, but not limited to, transduction with a gamma-retrovirus, a lentivirus, or a transposon system. There are a variety of suitable formulations of the pharmaceutical composition of the invention. Suitable formulations may include any of those for parenteral, subcutaneous, intravenous, intramuscular, intratumoral, intraarterial, intrathecal, or interperitoneal administration. More than one route can be used to administer the inventive CAR materials, and in certain instances, a particular route can provide a more immediate and more effective response than another route.

Preferably, the inventive CAR material is administered by injection, e.g., intravenously. When the inventive CAR material is a host cell expressing the inventive CARs (or functional variant thereof), the pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, Ill.), PLASMA-LYTE A (Baxter, Deerfield, Ill.), about 5% dextrose in water, or Ringer's lactate. In an embodiment, the pharmaceutically acceptable carrier is supplemented with human serum albumen.

The composition can employ time-released, delayed release, and sustained release delivery systems such that the delivery of the inventive composition occurs prior to, and with sufficient time to cause, sensitization of the site to be treated. Many types of release delivery systems are available and known to those of ordinary skill in the art. Such systems can avoid repeated administrations of the composition, thereby increasing convenience to the subject and the physician, and may be particularly suitable for certain composition embodiments of the invention.

Without being bound to a particular theory or mechanism, it is believed that by eliciting an antigen-specific response against CD19 and/or CD20, the first and/or second CARs provide for one or more of the following: targeting and destroying CD19 and/or CD20-expressing cancer cells, reducing or eliminating cancer cells, facilitating infiltration of immune cells to tumor site(s), and enhancing/extending anti-cancer responses.

It is contemplated that the first and/or second CARs materials can be used in methods of treating or preventing a disease, e.g., cancer, in a mammal. Without being bound to a particular theory or mechanism, the first and/or second CARs have biological activity, e.g., ability to recognize antigen, e.g., CD19 and/or CD20, such that the first and/or second CAR when expressed by a cell is able to mediate an immune response against the cell expressing the antigen, e.g., CD19 and/or CD20, for which the first and/or second CAR is specific. In this regard, an embodiment of the invention provides a method of treating or preventing cancer in a mammal, comprising administering to the mammal any of the CARs (including functional portions and variants thereof) (alone or in combination with a suicide gene product), nucleic acids, systems, protein(s) (including combination(s) of proteins) encoded by the nucleic acids, recombinant expression vectors, host cells (including populations thereof) and/or pharmaceutical compositions of the invention in an amount effective to treat or prevent cancer in the mammal. In a preferred embodiment, the method comprises infusing the mammal with host cells transduced with the inventive CAR construct.

One or more isolated host cells expressing the first and/or second CARs described herein can be contacted with a population of cancer cells that express CD19 and/or CD20 ex vivo, in vivo, or in vitro. “Ex vivo” refers to methods conducted within or on cells or tissue in an artificial environment outside an organism with minimum alteration of natural conditions. In contrast, the term “in vivo” refers to a method that is conducted within living organisms in their normal, intact state, while an “in vitro” method is conducted using components of an organism that have been isolated from its usual biological context. The inventive method preferably involves ex vivo and in vivo components. In this regard, for example, the isolated host cells described above can be cultured ex vivo under conditions to express the first and/or second CARs, and then directly transferred into a mammal (preferably a human) affected by a CD19 and/or CD20-positive cancer, e.g., lymphoma. Such a cell transfer method is referred to in the art as “adoptive cell transfer (ACT),” in which immune-derived cells are transferred into a recipient to transfer the functionality of the immune-derived cells to the host. The immune-derived cells may have originated from the recipient or from another individual. Adoptive cell transfer methods may be used to treat various types of cancers, including hematological cancers such as myeloma.

Once the composition comprising host cells expressing the inventive first and second CAR-encoding nucleic acid sequence, or a vector comprising the inventive first and second CAR-encoding nucleic acid sequence, is administered to a mammal (e.g., a human), the biological activity of the first and/or second CAR can be measured by any suitable method known in the art. In accordance with the inventive method, the first CAR binds to CD19 and/or the second CAR binds to CD20 on the cancer, and the cancer cells are destroyed. Binding of the first CAR to CD19 and/or the second CAR to CD20 on the surface of cancer cells can be assayed using any suitable method known in the art, including, for example, ELISA (enzyme-linked immunosorbent assays) and flow cytometry. The ability of the CARs to destroy cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). The biological activity of the first and/or second CAR also can be measured by assaying expression of certain cytokines, such as CD107a, IFNγ, IL-2, and TNF.

An embodiment of the invention further comprises lymphodepleting the mammal prior to administering the inventive CAR material. Examples of lymphodepletion include, but may not be limited to, nonmyeloablative lymphodepleting chemotherapy, myeloablative lymphodepleting chemotherapy, total body irradiation, etc. For example, a lymphodepleting chemotherapy regimen can be administered to the mammal prior to administering the inventive CAR material to the mammal. In an embodiment, cyclophosphamide and/or fludarabine are administered to a mammal prior to administering the inventive CAR material. In an embodiment, cyclophosphamide and/or fludarabine are administered for three consecutive days to a mammal prior to administering the inventive CAR material. In a further embodiment, cyclophosphamide is administered at a dose of from about 1 to about 100 mg/m² (e.g., from about 50 to about 950, from about 100 to about 900, from about 200 to about 800, from about 300 to about 700, from about 400 to about 600, from about 450 to about 550, from about 300 to about 500, about 300, about 400, or about 500 mg/m²). In a further embodiment, fludarabine is administered at a dose of from about 1 to about 100 mg/m² (e.g., from about 5 to about 80, from about 10 to about 70, from about 15 to about 60, from about 20 to about 50, from about 25 to about 40, from about 27 to about 33, or about 30 mg/m²). In some embodiments, the inventive CAR material can be administered (e.g., infused) about 72 hours after the last dose of chemotherapy.

For purposes of the inventive methods, wherein host cells or populations of cells are administered, the cells can be cells that are allogeneic or autologous to the mammal. Preferably, the cells are autologous to the mammal.

An “effective amount” or “an amount effective to treat” refers to a dose that is adequate to prevent or treat cancer in an individual. Amounts effective for a therapeutic or prophylactic use will depend on, for example, the stage and severity of the disease or disorder being treated, the age, weight, and general state of health of the patient, and the judgment of the prescribing physician. The size of the dose will also be determined by the particular CAR material selected, method of administration, timing and frequency of administration, the existence, nature, and extent of any adverse side-effects that might accompany the administration of a particular CAR material, and the desired physiological effect. It will be appreciated by one of skill in the art that various diseases or disorders (e.g., cancer) could require prolonged treatment involving multiple administrations, perhaps using the inventive CAR materials in each or various rounds of administration. By way of example and not intending to limit the invention, the dose of the inventive CAR material can be about 0.001 to about 1000 mg/kg body weight of the subject being treated/day, from about 0.01 to about 10 mg/kg body weight/day, about 0.01 mg to about 1 mg/kg body weight/day. In an embodiment of the invention, the dose may be from about 1×10⁴ to about 1×100 cells expressing the first and/or second CAR per kg body weight. When the inventive CAR material is a host cell, an exemplary dose of host cells may be a minimum of one million cells (1 million cells/dose to as many as 10¹¹ cells/dose), e.g., 1×10⁹ cells. When the inventive CAR material is a nucleic acid packaged in a virus, an exemplary dose of virus may be 1 ng/dose.

For purposes of the invention, the amount or dose of the inventive CAR material administered should be sufficient to effect a therapeutic or prophylactic response in the subject or animal over a reasonable time frame. For example, the dose of the inventive CAR material should be sufficient to bind to antigen, or detect, treat or prevent disease, e.g., cancer, in a period of from about 2 hours or longer, e.g., about 12 to about 24 or more hours, from the time of administration. In certain embodiments, the time period could be even longer. The dose will be determined by the efficacy of the particular inventive CAR material and the condition of the animal (e.g., human), as well as the body weight of the animal (e.g., human) to be treated.

For purposes of the invention, an assay, which comprises, for example, comparing the extent to which target cells are lysed and/or IFNγ is secreted by T cells expressing the first and/or second CAR upon administration of a given dose of such T cells to a mammal, among a set of mammals of which is each given a different dose of the T cells, could be used to determine a starting dose to be administered to a mammal. The extent to which target cells are lysed and/or IFNγ is secreted upon administration of a certain dose can be assayed by methods known in the art.

When the inventive CAR materials are administered with one or more additional therapeutic agents, one or more additional therapeutic agents can be coadministered to the mammal. By “coadministering” is meant administering one or more additional therapeutic agents and the inventive CAR materials sufficiently close in time such that the inventive CAR materials can enhance the effect of one or more additional therapeutic agents, or vice versa. In this regard, the inventive CAR materials can be administered first and the one or more additional therapeutic agents can be administered second, or vice versa. Alternatively, the inventive CAR materials and the one or more additional therapeutic agents can be administered simultaneously. An exemplary therapeutic agent that can be co-administered with the CAR materials is IL-2. It is believed that IL-2 enhances the therapeutic effect of the inventive CAR materials. Without being bound by a particular theory or mechanism, it is believed that IL-2 enhances therapy by enhancing the in vivo expansion of the numbers of cells expressing the first and/or second CARs.

The mammal referred to herein can be any mammal. As used herein, the term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes). Preferably, the mammal is a human.

With respect to the inventive methods, the cancer can be any cancer. In an embodiment of the invention, the cancer is a CD19 and/or CD20-expressing cancer. In an embodiment of the invention, the cancer is leukemia and/or lymphoma.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive method can include treatment or prevention of one or more conditions or symptoms of the disease, e.g., cancer, being treated or prevented. Also, for purposes herein, “prevention” can encompass delaying the onset of the disease, e.g., cancer, or a symptom or condition thereof or preventing the recurrence of the disease, e.g., cancer.

Another embodiment of the invention provides any of the first and/or second CARs (including functional portions and variants thereof) (alone or in combination with a suicide gene product), nucleic acids, systems, protein(s) (including combination(s) of proteins) encoded by the nucleic acids, recombinant expression vectors, host cells (including populations thereof) and/or pharmaceutical compositions described herein with respect to other aspects of the invention for use in a method of treating or preventing cancer in a mammal. Still another embodiment of the invention provides the use of any of the first and/or second CARs (including functional portions and variants thereof) (alone or in combination with a suicide gene product), nucleic acids, systems, protein(s) (including combination(s) of proteins) encoded by the nucleic acids, recombinant expression vectors, host cells (including populations thereof) and/or pharmaceutical compositions described herein with respect to other aspects of the invention in the manufacture of a medicament for the treatment or prevention of cancer in a mammal. The cancer may be any of the cancers described herein.

A further embodiment of the invention provides one or more polypeptide(s) encoded by the nucleic acids of the invention.

Another embodiment of the invention provides methods of detecting the presence of cancer in a mammal, comprising (a) contacting a sample comprising one or more cells from the mammal with nucleic acids, protein(s) (including combination(s) of proteins) encoded by the nucleic acids, recombinant expression vectors, host cells (including populations thereof) and/or pharmaceutical compositions of the invention, thereby forming a complex, and (b) detecting the complex, wherein detection of the complex is indicative of the presence of cancer in the mammal.

The following examples further illustrate the invention but, of course, should not be construed as in any way limiting its scope.

The following includes certain aspects of the invention.

1. A nucleic acid comprising a nucleotide sequence encoding a chimeric antigen receptor (CAR) construct comprising:

(a) a first CAR comprising

a first antigen binding domain,

a first transmembrane domain, and

a first intracellular T cell signaling domain;

(b) a second CAR comprising

a second antigen binding domain,

a second transmembrane domain, and

a second intracellular T cell signaling domain; and

(c) a cleavage sequence;
wherein the cleavage sequence is positioned between the first and second CARs,
wherein the first antigen binding domain of the first CAR has antigenic specificity for CD19, and
wherein the second antigen binding domain of the second CAR has antigenic specificity for CD20.

2. The nucleic acid according to aspect 1, wherein the cleavage sequence comprises any one of the following: porcine teschovirus-1 2A (P2A) amino acid sequence, equine rhinitis A virus (E2A) amino acid sequence, thosea asigna virus 2A (T2A) amino acid sequence, foot-and-mouth disease virus (F2A) amino acid sequence, or a furin-cleavable amino acid sequence, modified versions of any of the foregoing, or any combination of the foregoing.

3. The nucleic acid according to aspect 1 or 2, wherein the cleavage sequence comprises a foot-and-mouth disease virus (F2A) amino acid sequence.

4. The nucleic acid according to any one of aspects 1-3, wherein the cleavage sequence comprises an amino acid sequence comprising SEQ ID NO: 10.

5. The nucleic acid according to any one of aspects 1-4, wherein the first antigen binding domain comprises the six CDRs of Hu19.

6. The nucleic acid according to any one of aspects 1-5, wherein the first antigen binding domain comprises a first variable region comprising the amino acid sequence of SEQ ID NO: 4 and a second variable region comprising the amino acid sequence of SEQ ID NO: 6.

7. The nucleic acid according to any one of aspects 1-6, wherein the first antigen binding domain comprises single-chain variable fragment Hu19.

8. The nucleic acid according to any one of aspects 1-7, wherein the second antigen binding domain comprises the six CDRs of 11B8, C2B8, 2.1.2, 8G6, or GA101.

9. The nucleic acid according to any one of aspects 1-7, wherein the second antigen binding domain comprises an antigen binding domain of antibody C2B, 11B8, 8G6, 2.1.2, or GA101.

10. The nucleic acid according to any one of aspects 1-9, wherein one or both of the first and second transmembrane domain(s) comprises a CD8 transmembrane domain.

11. The nucleic acid according to any one of aspects 1-10, wherein one or both of the first and second CARs comprises a hinge domain.

12. The nucleic acid according to any one of aspects 1-11, wherein one or both of the first and second intracellular T cell signaling domain(s) comprises any one of the following: a human CD28 protein, a human CD3-zeta protein, a human FcRγ protein, a CD27 protein, an OX40 protein, a human 4-1BB protein, a human inducible T-cell costimulatory protein (ICOS), modified versions of any of the foregoing, or any combination of the foregoing.

13. The nucleic acid according to any one of aspects 1-12, wherein one or both of the first and second intracellular T cell signaling domain(s) comprises a CD28 intracellular T cell signaling sequence.

14. The nucleic acid according to aspect 13, wherein the CD28 intracellular T cell signaling sequence comprises the amino acid sequence of SEQ ID NO: 8.

15. The nucleic acid according to any one of aspects 1-14, wherein one or both of the first and second intracellular T cell signaling domain(s) comprises a CD3 zeta (ξ) intracellular T cell signaling sequence.

16. The nucleic acid according to aspect 15, wherein the CD3ξ intracellular T cell signaling sequence comprises the amino acid sequence of SEQ ID NO: 9.

17. The nucleic acid according to any one of aspects 1-16, wherein the CAR construct comprises a CD8 leader domain.

18. The nucleic acid according to aspect 17, wherein the CD8 leader domain sequence comprises the amino acid sequence of SEQ ID NO: 3.

19. The nucleic acid according to any one of aspects 1-18, wherein the CAR construct comprises exactly two CARs being the first and second CARs, respectively.

20. The nucleic acid of any one of aspects 1-19, which encodes a CAR construct comprising the amino acid sequence of any one of SEQ ID NOs: 2, 16, 20, 24, or 29.

21. One or more polypeptide(s) encoded by the nucleic acid of any one of aspects 1-20.

22. A recombinant expression vector comprising the nucleic acid of any one of aspects 1-20.

23. An isolated host cell comprising the recombinant expression vector of aspect 22.

24. A population of cells comprising at least one host cell of aspect 23.

25. A pharmaceutical composition comprising the nucleic acid of any one of aspects 1-20, the one or more polypeptide(s) of aspect 21, the recombinant expression vector of aspect 22, the host cell of aspect 23, or the population of cells of aspect 24, and a pharmaceutically acceptable carrier.

26. A method of detecting the presence of cancer in a mammal, comprising:

(a) contacting a sample comprising one or more cells from the mammal with the nucleic acid of any one of aspects 1-20, the one or more polypeptide(s) of aspect 21, the recombinant expression vector of aspect 22, the host cell of aspect 23, the population of cells of aspect 24, or the pharmaceutical composition of aspect 25, thereby forming a complex, and

(b) detecting the complex, wherein detection of the complex is indicative of the presence of cancer in the mammal.

27. The nucleic acid of any one of aspects 1-20, the one or more polypeptide(s) of aspect 21, the recombinant expression vector of aspect 22, the host cell of aspect 23, the population of cells of aspect 24, or the pharmaceutical composition of aspect 25 for use in the treatment or prevention of cancer in a mammal.

28. The host cell of aspect 23 or the population of cells of aspect 24 for the use of aspect 27.

29. The host cell of aspect 23 or the population of cells of aspect 24 for the use of aspect 27 or 28, wherein the host cell or population of cells is autologous in relation to the mammal.

30. The host cell of aspect 23 or the population of cells of aspect 24 for the use of aspect 27 or 28, wherein the host cell or population of cells is allogeneic in relation to the mammal.

31. The nucleic acid of any one of aspects 1-20, the one or more polypeptide(s) of aspect 21, the recombinant expression vector of aspect 22, the host cell of aspect 23, the population of cells of aspect 24, or the pharmaceutical composition of aspect 25, for the use of any one of aspects 27-30, wherein the cancer is a hematological malignancy.

The following examples further illustrate the invention but, of course, should not be construed as in any way limiting its scope.

### EXAMPLES

The following materials and methods were employed in the experiments described in Examples 1-18.

Cell Lines

K562 cells were transduced to express CD19 (CD19-K562) or low-affinity nerve growth factor (NFGR-K562) (Kochenderfer et al., J. Immunother., 32(7): 689-702 (2009)). K562 cells were also transduced to express CD20. The K562 trasductions were carried out by standard methods with the MSGV1 gamma-retroviral vector (Hughes, et al., Human Gene Therapy, 16(4): 457-472 (2005)). The NGFR-K562 cells served as CD19-negative control cells. CCRF-CEM cells (ATCC) also served as negative control cells. CD19⁺ NALM6 is an acute lymphoid leukemia cell line (DSMZ, Braunschweig, Germany). Toledo, ST486, and SU-DHL4 are all CD19⁺ cell lines (ATCC). ST486 null (CD19−/−) cell line had CD19 expression abrogated by CRISPR/Cas9. All of the human samples mentioned were obtained from patients enrolled in IRB-approved clinical trials at the National Cancer Institute.

### CAR Construction

Five bicistronic anti-CD19/anti-CD20 CARs constructs were designed. The sequence of each CAR followed this pattern from the N-terminus to the C-terminus: leader sequence (SS) (e.g., from human CD8α, an anti-CD19 antigen binding domain (e.g., a scFv made up from N-terminus to C-terminus of an anti-CD19 scFv comprising the heavy and light chains of an anti-CD19 antibody joined by a linker sequence), a human CD8α hinge and transmembrane domains, an intracellular T cell signaling domain of human CD28, an intracellular T cell signaling domain of human CD3ξ, a cleavage sequence that includes a F2A ribosomal skip sequence and a foot-and-mouth disease virus (F2A) amino acid sequence, an anti-CD20 antigen binding domain (e.g., a scFv made up from N-terminus to C-terminus of an anti-CD20 scFv comprising the heavy and light chains of an anti-CD20 antibody joined by a linker sequence), a CD8α hinge and transmembrane domains, an intracellular T cell signaling domain of human 4-1BB, and an intracellular T cell signaling domain of CD3ξ. The CARs are the same except that the CD20 antigen binding domains are created from different scFvs. ScFvs from antibodies 11B8, C2B8, 8G6-5, 2.1.2, and GA101 were used. The specific sequences of each component of the synthesized CAR constructs are below in Tables 1-5.

TABLE 1
Hu1928-11B8BB

| | SEQ | |
|---|---|---|
| | ID | |
| Description | NO: | Sequence |
| CD8α SS | 3 | MALPVTALLLPLALL |
| | | LHAARP |
| CD19 scFv: | | |
| LC | 4 | EIVLTQSPGTLSLSP |
| | | GERATLSCRASQSVS |
| | | SSYLAWYQQKPGQAP |
| | | RLLIYGASSRATGIP |
| | | DRFSGSGSGTDFTLT |
| | | ISRLEPEDFAVYYCQ |
| | | QYGSSRFTFGPGTKV |
| | | DIK |
| Linker | 5 | GSTSGSGKPGSGEGS |
| | | TKG |
| HC | 6 | QVQLVQSGAEVKKPG |
| | | SSVKVSCKDSGGTFS |
| | | SYAISWVRQAPGQGL |
| | | EWMGGIIPIFGTTNY |
| | | AQQFQGRVTITADES |
| | | TSTAYMELSSLRSED |
| | | TAVYYCAREAVAADW |
| | | LDPWGQGTLVTVSS |
| CD8α | 7 | FVPVFLPAKPTTTPA |
| | | PRPPTPAPTIASQPL |
| | | SLRPEACRPAAGGAV |
| | | HTRGLDFACDIYIWA |
| | | PLAGTCGVLLLSLVI |
| | | TLYCNHRN |
| CD28 | 8 | RSKRSRLLHSDYMNM |
| | | TPRRPGPTRKHYQPY |
| | | APPRDFAAYRS |
| CD3ζ | 9 | RVKFSRSADAPAYQQ |
| | | GQNQLYNELNLGRRE |
| | | EYDVLDKRRGRDPEM |
| | | GGKPRRKNPQEGLYN |
| | | ELQKDKMAEAYSEIG |
| | | MKGERRRGKGHDGLY |
| | | QGLSTATKDTYDALH |
| | | MQALPPR |
| Cleavage | 10 | RAKRSGSGAPVKQTL |
| sequence | | NFDLLKLAGDVESNP |
| | | GP |
| CD20 scFv: | | |
| LC | 11 | EIVLTQSPATLSLSP |
| | | GERATLSCRASQSVS |
| | | SYLAWYQQKPGQAPR |
| | | LLIYDASNRATGIPA |
| | | RFSGSGSGTDFTLTI |
| | | SSLEPEDFAVYYCQQ |
| | | RSDWPLTFGGGTKVE |
| | | IK |
| Linker | 12 | GGGGSGGGGSGGGGS |
| HC | 13 | EVQLVQSGGGLVHPG |
| | | GSLRLSCTGSGFTFS |
| | | YHAMHWVRQAPGKGL |
| | | EWVSIIGTGGVTYYA |
| | | DSVKGRFTISRDNVK |
| | | NSLYLQMNSLRAEDM |
| | | AVYYCARDYYGAGSF |
| | | YDGLYGMDVWGQGTT |
| | | VTVSS |
| CD8α | 7 | FVPVFLPAKPTTTPA |
| | | PRPPTPAPTIASQPL |
| | | SLRPEACRPAAGGAV |
| | | HTRGLDFACDIYIWA |
| | | PLAGTCGVLLLSLVI |
| | | TLYCNHRN |
| 4-1BB | 14 | KRGRKKLLYIFKQPF |
| | | MRPVQTTQEEDGCSC |
| | | RFPEEEEGGCEL |
| CD3ζ | 9 | RVKFSRSADAPAYQQ |
| | | GQNQLYNELNLGRRE |
| | | EYDVLDKRRGRDPEM |
| | | GGKPRRKNPQEGLYN |
| | | ELQKDKMAEAYSEIG |
| | | MKGERRRGKGHDGLY |
| | | QGLSTATKDTYDALH |
| | | MQALPPR |

TABLE 2
Hu1928-C2B8BB

| | SEQ | |
|---|---|---|
| | ID | |
| Description | NO: | Sequence |
| CD8α SS | 3 | MALPVTALLLPLALL |
| | | LHAARP |
| CD19 scFv: | | |
| LC | 4 | EIVLTQSPGTLSLSP |
| | | GERATLSCRASQSVS |
| | | SSYLAWYQQKPGQAP |
| | | RLLIYGASSRATGIP |
| | | DRFSGSGSGTDFTLT |
| | | ISRLEPEDFAVYYCQ |
| | | QYGSSRFTFGPGTKV |
| | | DIK |
| Linker | 5 | GSTSGSGKPGSGEGS |
| | | TKG |
| HC | 6 | QVQLVQSGAEVKKPG |
| | | SSVKVSCKDSGGTFS |
| | | SYAISWVRQAPGQGL |
| | | EWMGGIIPIFGTTNY |
| | | AQQFQGRVTITADES |
| | | TSTAYMELSSLRSED |
| | | TAVYYCAREAVAADW |
| | | LDPWGQGTLVTVSS |
| CD8α | 7 | FVPVFLPAKPTTTPA |
| | | PRPPTPAPTIASQPL |
| | | SLRPEACRPAAGGAV |
| | | HTRGLDFACDIYIWA |
| | | PLAGTCGVLLLSLVI |
| | | TLYCNHRN |
| CD28 | 8 | RSKRSRLLHSDYMNM |
| | | TPRRPGPTRKHYQPY |
| | | APPRDFAAYRS |
| CD3ζ | 9 | RVKFSRSADAPAYQQ |
| | | GQNQLYNELNLGRRE |
| | | EYDVLDKRRGRDPEM |
| | | GGKPRRKNPQEGLYN |
| | | ELQKDKMAEAYSEIG |
| | | MKGERRRGKGHDGLY |
| | | QGLSTATKDTYDALH |
| | | MQALPPR |
| Cleavage | 10 | RAKRSGSGAPVKQTL |
| sequence | | NFDLLKLAGDVESNP |
| | | GP |
| CD20 scFv: | | |
| LC | 17 | QIVLSQSPAILSASP |
| | | GEKVTMTCRASSSVS |
| | | YIHWFQQKPGSSPKP |
| | | WIYATSNLASGVPVR |
| | | FSGSGSGTSYSLTIS |
| | | RVEAEDAATYYCQQW |
| | | TSNPPTFGGGTKLEI |
| | | K |
| Linker | 12 | GGGGSGGGGSGGGGS |
| HC | 18 | QVQLQQPGAELVKPG |
| | | ASVKMSCKASGYTFT |
| | | SYNMHWVKQTPGRGL |
| | | EWIGAIYPGNGDTSY |
| | | NQKFKGKATLTADKS |
| | | SSTAYMQLSSLTSED |
| | | SAVYYCARSTYYGGD |
| | | WYFNVWGAGTTVTVS |
| | | A |
| CD8α | 7 | FVPVFLPAKPTTTPA |
| | | PRPPTPAPTIASQPL |
| | | SLRPEACRPAAGGAV |
| | | HTRGLDFACDIYIWA |
| | | PLAGTCGVLLLSLVI |
| | | TLYCNHRN |
| 4-1BB | 14 | KRGRKKLLYIFKQPF |
| | | MRPVQTTQEEDGCSC |
| | | RFPEEEEGGCEL |
| CD3ζ | 9 | RVKFSRSADAPAYQQ |
| | | GQNQLYNELNLGRRE |
| | | EYDVLDKRRGRDPEM |
| | | GGKPRRKNPQEGLYN |
| | | ELQKDKMAEAYSEIG |
| | | MKGERRRGKGHDGLY |
| | | QGLSTATKDTYDALH |
| | | MQALPPR |

TABLE 3
Hu1928-2.1.2BB

| | SEQ | |
|---|---|---|
| | ID | |
| Description | NO: | Sequence |
| CD8α SS | 3 | MALPVTALLLPLAL |
| | | LLHAARP |
| CD19 scFv: | | |
| LC | 4 | EIVLTQSPGTLSLSP |
| | | GERATLSCRASQSVS |
| | | SSYLAWYQQKPGQAP |
| | | RLLIYGASSRATGIP |
| | | DRFSGSGSGTDFTLT |
| | | ISRLEPEDFAVYYCQ |
| | | QYGSSRFTFGPGTKV |
| | | DIK |
| Linker | 5 | GSTSGSGKPGSGEGS |
| | | TKG |
| HC | 6 | QVQLVQSGAEVKKPG |
| | | SSVKVSCKDSGGTFS |
| | | SYAISWVRQAPGQGL |
| | | EWMGGIIPIFGTTNY |
| | | AQQFQGRVTITADES |
| | | TSTAYMELSSLRSED |
| | | TAVYYCAREAVAADW |
| | | LDPWGQGTLVTVSS |
| CD8α SS | 3 | MALPVTALLLPLAL |
| | | LLHAARP |
| CD19 scFv: | | |
| CD8α | 7 | FVPVFLPAKPTTTP |
| | | APRPPTPAPTIASQ |
| | | PLSLRPEACRPAAG |
| | | GAVHTRGLDFACDI |
| | | YIWAPLAGTCGVLL |
| | | LSLVITLYCNHRN |
| CD28 | 8 | RSKRSRLLHSDYMN |
| | | MTPRRPGPTRKHYQ |
| | | PYAPPRDFAAYRS |
| CD3ζ | 9 | RVKFSRSADAPAYQ |
| | | QGQNQLYNELNLGR |
| | | REEYDVLDKRRGRD |
| | | PEMGGKPRRKNPQE |
| | | GLYNELQKDKMAEA |
| | | YSEIGMKGERRRGK |
| | | GHDGLYQGLSTATK |
| | | DTYDALHMQALPPR |
| Cleavage | 10 | RAKRSGSGAPVKQT |
| sequence | | LNFDLLKLAGDVES |
| | | NPGP |
| CD20 scFv: | | |
| LC | 21 | DIVMTQTPHSSPVTL |
| | | GQPASISCRSSQSLV |
| | | SRDGNTYLSWLQQRP |
| | | GQPPRLLIYKISNRF |
| | | SGVPNRFSGSGAGTD |
| | | FTLKISRVKAEDVGV |
| | | YYCMQATQFPLTFGQ |
| | | GTRLEIK |
| Linker | 12 | GGGGSGGGGSGGGGS |
| HC | 22 | EVQLVQSGAEVKKPG |
| | | ESLKISCKGSGYSFT |
| | | SYWIGWVRQMPGKGL |
| | | EWMGIIYPGDSDTRY |
| | | SPSFQGQVTISADKS |
| | | ISTAYLQWSSLKASD |
| | | TAMYYCARQGDFWSG |
| | | YGGMDVWGQGTTVTV |
| | | SS |
| CD8α | 7 | FVPVFLPAKPTTTPA |
| | | PRPPTPAPTIASQPL |
| | | SLRPEACRPAAGGAV |
| | | HTRGLDFACDIYIWA |
| | | PLAGTCGVLLLSLVI |
| | | TLYCNHRN |
| 4-1BB | 14 | KRGRKKLLYIFKQPF |
| | | MRPVQTTQEEDGCSC |
| | | RFPEEEEGGCEL |
| CD3ζ | 9 | RVKFSRSADAPAYQQ |
| | | GQNQLYNELNLGRRE |
| | | EYDVLDKRRGRDPEM |
| | | GGKPRRKNPQEGLYN |
| | | ELQKDKMAEAYSEIG |
| | | MKGERRRGKGHDGLY |
| | | QGLSTATKDTYDALH |
| | | MQALPPR |

TABLE 4
Hu1928-8G6-5BB

| | SEQ | |
|---|---|---|
| | ID | |
| Description | NO: | Sequence |
| CD8α SS | 3 | MALPVTALLLPLAL |
| | | LLHAARP |
| CD19 scFv: | | |
| LC | 4 | EIVLTQSPGTLSLS |
| | | PGERATLSCRASQS |
| | | VSSSYLAWYQQKPG |
| | | QAPRLLIYGASSRA |
| | | TGIPDRFSGSGSGT |
| | | DFTLTISRLEPEDF |
| | | AVYYCQQYGSSRFT |
| | | FGPGTKVDIK |
| Linker | 5 | GSTSGSGKPGSGEG |
| | | STKG |
| HC | 6 | QVQLVQSGAEVKKP |
| | | GSSVKVSCKDSGGT |
| | | FSSYAISWVRQAPG |
| | | QGLEWMGGIIPIFG |
| | | TTNYAQQFQGRVTI |
| | | TADESTSTAYMELS |
| | | SLRSEDTAVYYCAR |
| | | EAVAADWLDPWGQG |
| | | TLVTVSS |
| CD8α | 7 | FVPVFLPAKPTTTP |
| | | APRPPTPAPTIASQ |
| | | PLSLRPEACRPAAG |
| | | GAVHTRGLDFACDI |
| | | YIWAPLAGTCGVLL |
| | | LSLVITLYCNHRN |
| CD28 | 8 | RSKRSRLLHSDYMN |
| | | MTPRRPGPTRKHYQ |
| | | PYAPPRDFAAYRS |
| CD3ζ | 9 | RVKFSRSADAPAYQ |
| | | QGQNQLYNELNLGR |
| | | REEYDVLDKRRGRD |
| | | PEMGGKPRRKNPQE |
| | | GLYNELQKDKMAEA |
| | | YSEIGMKGERRRGK |
| | | GHDGLYQGLSTATK |
| | | DTYDALHMQALPPR |
| Cleavage | 10 | RAKRSGSGAPVKQT |
| skip | | LNFDLLKLAGDVES |
| sequence | | NPGP |
| CD20 scFv: | | |
| LC | 25 | EIVMTQSPATLSMS |
| | | PGERATLSCRASQS |
| | | VSRNLAWYQQKVGQ |
| | | APRLLISGASTRAT |
| | | GIPARFSGSGSGTE |
| | | FTLTINSLQSEDFA |
| | | VYYCQQSNDWPLTF |
| | | GQGTRLEIK |
| Linker | 12 | GGGGSGGGGSGGGG |
| | | S |
| HC | 26 | EVQLAESGGDLVQS |
| | | GRSLRLSCAASGIT |
| | | FHDYAMHWVRQPPG |
| | | KGLEWVSGISWNSD |
| | | YIGYADSVKGRFTI |
| | | SRDNAKKSLYLQMN |
| | | SLRPDDTALYYCVK |
| | | DFHYGSGSNYGMDV |
| | | WGQGTTVTVSS |
| CD8α SS | 3 | MALPVTALLLPLAL |
| | | LLHAARP |
| CD19 scFv: | | |
| CD8α | 7 | FVPVFLPAKPTTTP |
| | | APRPPTPAPTIASQ |
| | | PLSLRPEACRPAAG |
| | | GAVHTRGLDFACDI |
| | | YIWAPLAGTCGVLL |
| | | LSLVITLYCNHRN |
| 4-1BB | 14 | KRGRKKLLYIFKQP |
| | | FMRPVQTTQEEDGC |
| | | SCRFPEEEEGGCEL |
| CD3ζ | 9 | RVKFSRSADAPAYQ |
| | | QGQNQLYNELNLGR |
| | | REEYDVLDKRRGRD |
| | | PEMGGKPRRKNPQE |
| | | GLYNELQKDKMAEA |
| | | YSEIGMKGERRRGK |
| | | GHDGLYQGLSTATK |
| | | DTYDALHMQALPPR |

TABLE 5
Hu1928-GA101BB

| | SEQ | |
|---|---|---|
| | ID | |
| Description | NO: | Sequence |
| CD8α SS | 3 | MALPVTALLLPLAL |
| | | LLHAARP |
| CD19 scFv: | | |
| LC | 4 | EIVLTQSPGTLSLS |
| | | PGERATLSCRASQS |
| | | VSSSYLAWYQQKPG |
| | | QAPRLLIYGASSRA |
| | | TGIPDRFSGSGSGT |
| | | DFTLTISRLEPEDF |
| | | AVYYCQQYGSSRFT |
| | | FGPGTKVDIK |
| Linker | 5 | GSTSGSGKPGSGEG |
| | | STKG |
| HC | 6 | QVQLVQSGAEVKKP |
| | | GSSVKVSCKDSGGT |
| | | FSSYAISWVRQAPG |
| | | QGLEWMGGIIPIFG |
| | | TTNYAQQFQGRVTI |
| | | TADESTSTAYMELS |
| | | SLRSEDTAVYYCAR |
| | | EAVAADWLDPWGQG |
| | | TLVTVSS |
| CD8α | 7 | FVPVFLPAKPTTTP |
| | | APRPPTPAPTIASQ |
| | | PLSLRPEACRPAAG |
| | | GAVHTRGLDFACDI |
| | | YIWAPLAGTCGVLL |
| | | LSLVITLYCNHRN |
| CD28 | 8 | RSKRSRLLHSDYMN |
| | | MTPRRPGPTRKHYQ |
| | | PYAPPRDFAAYRS |
| CD3ζ | 9 | RVKFSRSADAPAYQ |
| | | QGQNQLYNELNLGR |
| | | REEYDVLDKRRGRD |
| | | PEMGGKPRRKNPQE |
| | | GLYNELQKDKMAEA |
| | | YSEIGMKGERRRGK |
| | | GHDGLYQGLSTATK |
| | | DTYDALHMQALPPR |
| CD8α SS | 3 | MALPVTALLLPLAL |
| | | LLHAARP |
| Cleavage | 10 | RAKRSGSGAPVKQT |
| sequence | | LNFDLLKLAGDVES |
| | | NPGP |
| CD20 scFv: | | |
| LC | 29 | DIVMTQTPLSLPVT |
| | | PGEPASISCRSSKS |
| | | LLHSNGITYLYWYL |
| | | QKPGQSPQLLIYQM |
| | | SNLVSGVPDRFSGS |
| | | GSGTDFTLKISRVE |
| | | AEDVGVYYCAQNLE |
| | | LPYTFGGGTKVEIK |
| Linker | 12 | GGGGSGGGGSGGGG |
| | | S |
| HC | 30 | QVQLVQSGAEVKKP |
| | | GSSVKVSCKASGYA |
| | | FSYSWINWVRQAPG |
| | | QGLEWMGRIFPGDG |
| | | DTDYNGKFKGRVTI |
| | | TADKSTSTAYMELS |
| | | SLRSEDTAVYYCAR |
| | | NVFDGYWLVYWGQG |
| | | TLVTVSS |
| CD8α | 7 | FVPVFLPAKPTTTP |
| | | APRPPTPAPTIASQ |
| | | PLSLRPEACRPAAG |
| | | GAVHTRGLDFACDI |
| | | YIWAPLAGTCGVLL |
| | | LSLVITLYCNHRN |
| 4-1BB | 14 | KRGRKKLLYIFKQP |
| | | FMRPVQTTQEEDGC |
| | | SCRFPEEEEGGCEL |
| CD3ζ | 9 | RVKFSRSADAPAYQ |
| | | QGQNQLYNELNLGR |
| | | REEYDVLDKRRGRD |
| | | PEMGGKPRRKNPQE |
| | | GLYNELQKDKMAEA |
| | | YSEIGMKGERRRGK |
| | | GHDGLYQGLSTATK |
| | | DTYDALHMQALPPR |

The anti-CD19 CAR, Hu19-CD828Z, containing variable region sequences of a fully-human antibody, a CD28 costimulatory domain, and a CD3ξ T-cell activation domain was used (Alabanza, et al., Molecular Therapy, 25(11): 2452-2465 (2017)). A scFv designated Hu19 was designed containing a light chain variable region (SEQ ID NO: 4), a linker peptide (GSTSGSGKPGSGEGSTKG [SEQ ID NO: 5]), and a heavy chain variable region (SEQ ID NO: 6). The scFv also included a human CD8α leader sequence (SEQ ID NO: 3). A DNA sequence encoding a CAR with the following components from 5′ to 3′ was designed: Hu19 scFv, part of the hinge region and the transmembrane region of the human CD8α molecule (SEQ ID NO: 7), the intracellular T cell signaling domain of the human CD28 (SEQ ID NO: 8), and the intracellular T cell signaling domain of human CD3ξ (SEQ ID NO: 9). The DNA sequence was synthesized using Invitrogen GENEAR™ Gene Synthesis (ThermoFisher Scientific) and named CAR Hu19-CD828Z. The Hu19-CD828Z sequence was inserted into the MSGV1 gamma-retroviral backbone to form MSGV1-Hu19-CD828Z using standard methods (Hughes, et al., Human Gene Therapy, 16: 457-72, (2005)).

To form a construct with the ability to recognize both CD19 and CD20, Hu19-CD828Z was incorporated into bicistronic constructs also encoding a separate CAR targeting CD20. Five anti-CD20 CAR constructs were made. The first construct included the CD8α leader sequence followed by the Hu19-CD828Z CAR sequence as described above. Next, an F2A-containing ribosomal skip cleavage sequence was added, followed one of the five anti-CD20 CARs. One of the anti-CD20 CARs was designated C2B8-CD8BBZ. This CAR contained CD8α leader sequence followed by a scFv made up of the C2B8 heavy and light chain variable regions linked by a linker made up of 3 repeats of 4 glycines and 1 serine (G4S)³. The wild-type murine C2B8 variable region sequences were used. C2B8 is also known as rituximab. After the scFv, CD8α hinge and transmembrane domains were added followed by the intracellular T cell signaling domains of human 4-1BB and human CD3ξ. The entire CAR construct including the Hu19-CD828Z and C2B8-CD8BBZ components with an intervening F2A-containing sequence was designated Hu1928-C2B8BB. The DNA sequence encoding Hu1928-C2B8BB was synthesized and cloned into the MSGV1 gamma-retroviral backbone.

As noted above, four more CAR constructs were designed and synthesized as described above. These variable regions used to create the scFv regions came from one of 3 fully-human antibodies, 11B8, 2.1.2, or 8G6-5, and one CAR had variable regions from the humanized antibody GA101. The anti-CD20 CARs were designated 11B8BB, 8G6-5BB, 2.1.2BB, and GA101BB. These CARs all had identical sequences except for their different scFvs. In each case, the variable regions were linked by a (G4S)³ linker.

Four bicistronic CAR constructs, Hu1928-11B8BB, Hu1928-2.1.2BB, Hu1928-8G6-5BB, Hu1928-GA101BB were synthesized by using the same process (see Tables 2-5). A fragment encoding the following components from 5′ to 3′ was synthesized by Invitrogen GENEART™ Gene Synthesis: BlpI restriction site, part of CD8α hinge and transmembrane domains, a CD28 moiety, the intracellular T cell signaling domain of CD3c, a furin site, a 4 amino acid spacer (SGSG [SEQ ID NO: 50]), an F2A site, CD8α leader sequence, anti-CD20 light chain variable region, (G4S)³ linker, anti-CD20 heavy chain variable region, CD8α hinge and transmembrane domains, 4-1BB moiety, CD3ξ intracellular T cell signaling domain, and finally, a SnaBI restriction site. This DNA fragment was ligated into BlpI/SnaBI-digested Hu1928_C2B8BB.

Five anti-CD20 CARs with the anti-CD20 C2B8 scFv were created to serve as controls in experiments. C2B8-CD828Z contains a CD28 costimulatory domain. The other anti-CD20 CARs contain 4-1BB costimulatory domains, these CARs also all include a CD8α leader sequence and CD3ξ T-cell activation domain. These CARs were all components of the bicistronic CAR constructs described above, and they were designed and constructed as described above. These CARs had one of five scFvs: C2B8, 11B8, 8G6-5, 2.1.2, and GA101. The CARs containing each of these CARs were C2B8-CD8BBZ, 11B8-CD8BBZ, 8G6-5-CD8BBZ, 2.1.2-CD8BBZ, and GA101-CD8BBZ.

### T-Cell Culture

PBMC were thawed and washed in T cell medium that contained AIM V™ medium (Invitrogen) plus 5% AB serum (Valley Biomedical, Winchester, Va.), 100 U/mL penicillin, and 100 μg/mL streptomycin. Prior to transductions, PBMC were suspended at a concentration of 1×10⁶ cells/mL in T cell medium plus 50 ng/mL of the anti-CD3 monoclonal antibody OKT3 (Ortho, Bridgewater, N.J.) and 300 IU/mL of IL-2. After transductions, T cells were maintained in T-cell medium plus IL-2.

### Gamma-Retroviral Transductions

To produce replication-incompetent gamma-retroviruses, packaging cells were transfected with plasmids encoding CARs along with a plasmid encoding the RD 114 envelope protein (see Kochenderfer et al., J. Immunother., 32(7): 689-702 (2009)). Gamma retroviral transduction of T cells was performed 2 days after initiation of T-cell cultures.

### CAR Detection on T Cells

An APC-labeled antibody designated Kip-1 that specifically binds to the linker component of the Hu19-CD828Z CAR was used to detect this CAR and C2B8-CD828Z. A commercially available anti-rituxumab antibody was used to detect C2B8-containing CARs other than C2B8-CD828Z. A PE-labeled antibody designated Kip-4 was used to detect anti-CD20 CARs. Kip-4 binds to the (G4S)³ linker. Staining for CD3, CD4, and CD8 was performed by using standard methods. Flow cytometry was performed by standard methods (i.e., FLOWJO™ software, Tree Star, Inc., Ashland, Oreg.). Dead cells were excluded by using 7-AAD (BD Biosciences).

### Interferon-Gamma and Tumor Necrosis Factor Alpha ELISAs

One-hundred thousand BCMA⁺ or BCMA-negative target cells were combined with 100,000 CAR-transduced T cells in duplicate wells of a 96 well round bottom plate in 200 μL of AIM V™ medium (Invitrogen) plus 5% human serum. The plates were incubated at 37° C. for 18-20 hours. Following the incubation, ELISAs for IFNγ were performed by using standard methods. Soluble BCMA protein (ORIGENE™) was added to some ELISAs at the start of the co-culture to determine if soluble BCMA had an impact on the ability of CAR T cells to recognize the targets.

### CD107a Assay

For each T cell culture that was tested, two tubes were prepared. One tube contained BCMA-K562 cells, and the other tube contained NGFR-K562 cells. Both tubes contained CAR-transduced T cells, 1 ml of AIM V™ medium (Invitrogen) plus 5% human AB serum, a titrated concentration of an anti-CD107a antibody (eBioscience, clone eBioH4A3, ThermoFisher Scientific), and 1 μL of GOLGISTOP™ (a protein transport inhibitor containing monensin, BD Biosciences). All tubes were incubated at 37° C. for 4 hours and then stained for CD3, CD4, and CD8.

### Proliferation Assays

Cocultures were set up in 24-well plates. Target cells included in cocultures were either 0.5×10⁶ irradiated BCMA-K562 cells or 0.5×10⁶ irradiated NGFR-K562 cells. The cocultures also included 1×10⁶ T cells from cultures that had been transduced with either anti-bcma2 or SP6. The T cells were labeled with carboxyfluorescein diacetate succinimidyl ester (CFSE, Invitrogen) as previously described (see, e.g., Mannering, et al., J. Immunol. Methods, 283: 173-183 (2003)). The medium used in the cocultures was AIM V^{T}M (Invitrogen) plus 5% human AB serum. IL-2 was not added to the medium. Four days after initiation, the live cells in each coculture were counted with trypan blue for dead cell exclusion, and flow cytometry was performed by Protein L staining.

### Cytotoxicity Assays

Cytotoxicity assays were conducted as previously described (see Kochenderfer et al., J. Immunother., 32(7): 689-702 (2009)). Cytotoxicity was measured by comparing survival of BMCA⁺ target cells relative to the survival of negative-control CCRF-CEM cells. Both of these cell types were combined in the same tubes with CAR-transduced T cells. CCRF-CEM negative control cells were labeled with the fluorescent dye 5-(and-6)-(((4-chloromethyl)benzoyl)amino) tetramethylrhodamine (CMTMR) (Invitrogen), and BMCA⁺ target cells were labeled with CFSE. Cocultures were set up in sterile 5 mL test tubes (BD Biosciences) in duplicate at multiple T cell to target cell ratios. The target cells contained in the tubes were 50,000 BMCA⁺ target cells along with 50,000 CCRF-CEM negative-control cells. The cultures were incubated for 4 hours at 37° C. Immediately after the incubation, 7AAD (7-amino-actinomycin D) (BD Biosciences) was added, and flow cytometry acquisition was performed. For each T cell plus target-cell culture, the percent survival of BMCA⁺ target cells was determined by dividing the percent live BMCA⁺ cells by the percent live CCRF-CEM negative control cells. The corrected percent survival of BMCA⁺ target cells was calculated by dividing the percent survival of BMCA⁺ target cells in each T cell plus target cell culture by the ratio of the percent live BMCA⁺ target cells to percent live CCRF-CEM negative-control cells in tubes containing only BMCA⁺ target cells and CCRF-CEM cells without effector T cells. This correction was necessary to account for variation in the starting cell numbers and for spontaneous target cell death. Cytotoxicity was calculated as follows: the percent cytotoxicity of BMCA⁺ target cells=100-corrected percent survival of BMCA⁺ target cells.

### Example 1

This example illustrates the preparation of bicistronic constructs that encode first and second CARs that target CD19 and CD20, respectively.

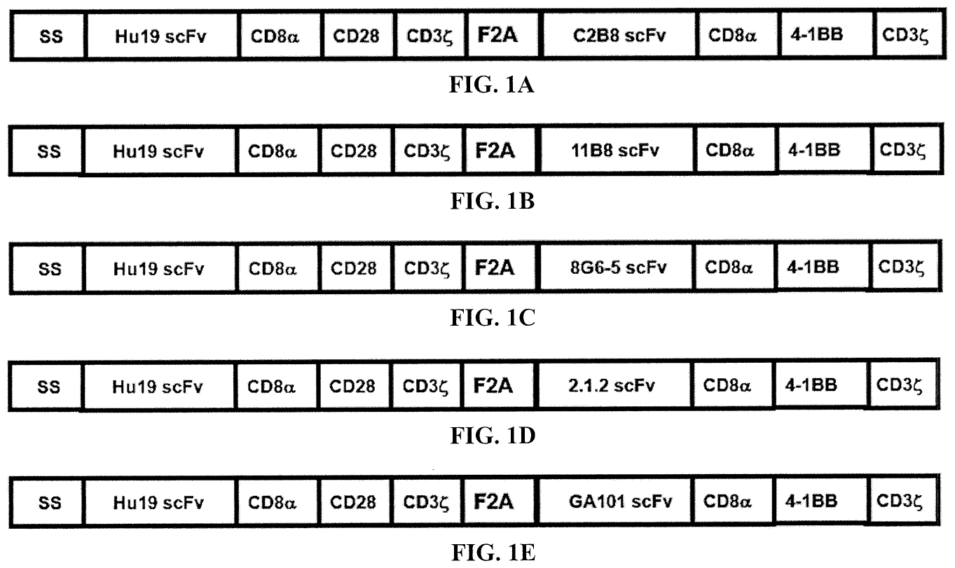

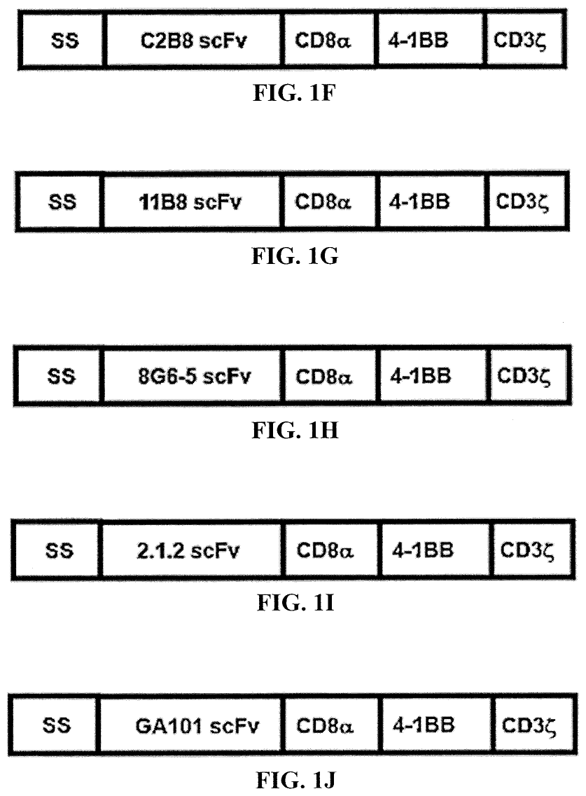

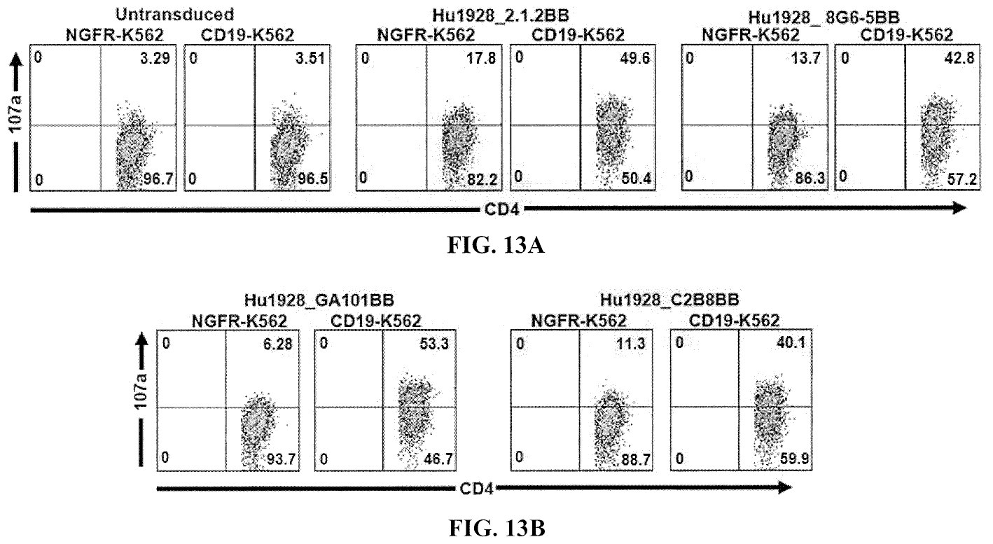

Bicistronic constructs were constructed as indicated above (see also FIGS. 1A-1J and 19). The CAR constructs were expressed with a gamma-retroviral vector. FIG. 2D shows T-cell expression of CAR Hu1928-C2B8BB (the CAR illustrated in FIG. 1A). FIG. 2A is the plot from the untransduced control. FIGS. 2B and 2C are the plots from CARs Hu19-CD828Z (anti-CD19 CAR) and C2B8-CD828Z (anti-CD20 CAR), respectively.

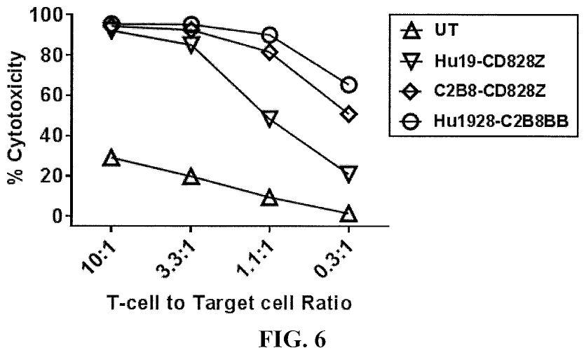

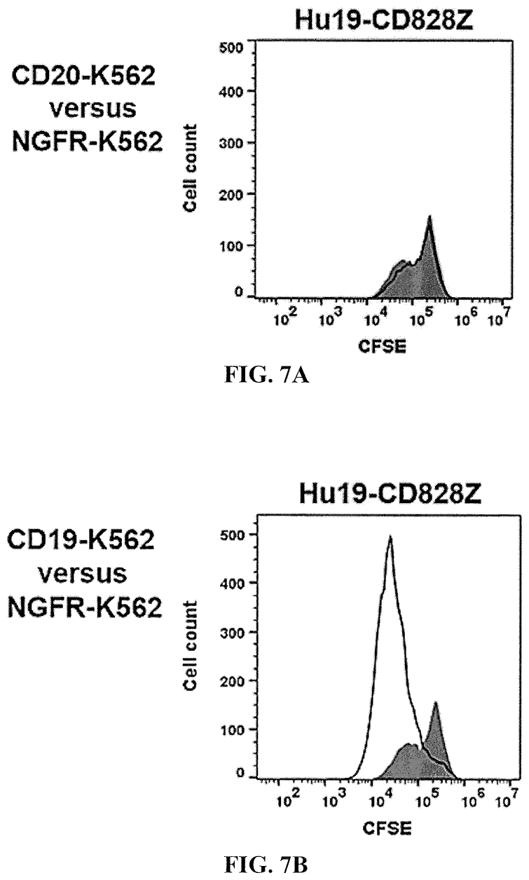

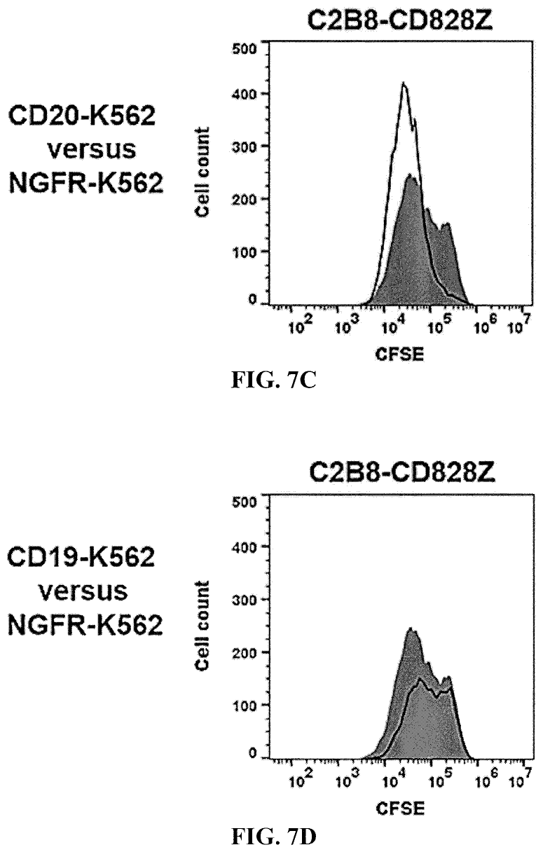

FIGS. 8A and 8B show CAR T-cell surface expression of Hu19-CD828Z, C2B8-CD8BBZ, Hu1928-C2B8BB, and Hu1928-11B8BB. FIG. 8A shows staining with the anti-Hu19 antibody, which binds to the linker included in Hu19-CD828Z. Hu19-CD828Z bound to all T cells transduced with constructs including the Hu19-CD828Z CAR. FIG. 8B shows staining with an anti-rituximab antibody that binds to C2B8. The anti-rituximab antibody bound to the CAR constructs that contain C2B8.

### Example 2

This example demonstrates that the first and second CARs encoded by the bicistronic constructs specifically recognize CD19 and CD20, respectively.

The CARs described in Example 1 were analyzed and it was found that they successfully triggered antigen-specific release of cytokines, as indicated below in Tables 6-9. The tables show that the indicated CARs were expressed on the surface of CAR T cells. Tables 6, 8, and 9 show that high levels of IFNγ were produced when the CAR T cells were cultured with target cells and that very low levels of IFNγ were produced when the CAR T cells were cultured with BAMC-negative target cells. CAR-expressing T cells cultured alone produced very low levels of IFNγ. Similarly, Table 7 shows that high levels of IL-2 were produced when the CAR T cells were cultured with target cells and that very low levels of IL-2 were produced when the CAR T cells were cultured with BAM/C-negative target cells.

TABLE 6
Antigen-specific IFNγ production after overnight co-culture with target cells

| | | | | Target cells | | | | | | |
|---|---|---|---|---|---|---|---|---|---|---|
| | | | | | | SU- | | | T- | |
| | CD19- | CD20- | | | | DHL- | NGFR- | CCRF- | cells | % CAR |
| T cells | K562 | K562 | Nalm6 | Toledo | ST486 | 4 | K562 | CEM | alone | + |
| UT | 347 | 305 | 29 | 59 | 135 | 41 | 330 | 21 | 16 | 0.3 |
| Hu19- | 21172 | 43 | 6941 | 4573 | 745 | 2279 | 241 | 25 | 26 | 59.5 |
| CD828Z | | | | | | | | | | |
| C2B8- | 1054 | 56741 | 1289 | 7767 | 14262 | 15870 | 735 | 440 | 512 | 77.1 |
| CD828Z | | | | | | | | | | |
| Hu1928- | 21473 | 35285 | 8690 | 13577 | 10321 | 11361 | 260 | 61 | 72 | 66.3 |
| C2B8BB | | | | | | | | | | |

All values are IFNγ in pg/ml except the last column, which is the percentage of each culture that expressed the Indicated CAR by flow cytometry.
Nalm6, Toledo, ST486 all express both CD19 and CD20
NGFR-K562 and CCRF-CEM lack both CD19 and CD20

TABLE 7
Antigen-specific IL-2 production after overnight co-culture with target cells (Patient 1)

| | | | | Target cells | | | | | | |
|---|---|---|---|---|---|---|---|---|---|---|
| | | | | | | SU- | | | T- | |
| | CD19- | CD20- | | | | DHL- | NGFR- | CCRF- | cells | % CAR |
| T cells | K562 | K562 | Nalm6 | Toledo | ST486 | 4 | K562 | CEM | alone | + |
| Untransduced | <16 | <16 | <16 | <16 | <16 | <16 | <16 | <16 | <16 | |
| Hu19- | 616 | <16 | 111 | 55 | <16 | <16 | <16 | <16 | <16 | |
| CD828Z | | | | | | | | | | |
| C2B8- | <16 | 4625 | <16 | 104 | 215 | 353 | <16 | <16 | <16 | |
| CD828Z | | | | | | | | | | |
| Hu1928- | 704 | 1424 | 161 | 403 | 129 | 175 | <16 | <16 | <16 | |
| C2B8BB | | | | | | | | | | |

All values are IL-2 in pg/ml except the last column, which is the percentage of each culture that expressed the Indicated CAR by flow cytometry.
Nalm6, Toledo, ST486 all express both CD19 and CD20
NGFR-K562 and CCRF-CEM lack both CD19 and CD20

TABLE 8
Antigen-specific IFNγ production after overnight co-culture with target cells (Patient 2)

| | | | | Target cells | | | | | | |
|---|---|---|---|---|---|---|---|---|---|---|
| | | | | | | SU- | | | T- | |
| | CD19- | CD20- | | | | DHL- | NGFR- | CCRF- | cells | % CAR |
| T cells | K562 | K562 | Nalm6 | Toledo | ST486 | 4 | K562 | CEM | alone | + |
| Untransduced | 101 | 211 | 161 | 251 | 214 | 197 | 66 | 57 | 56 | 0.2 |
| Hu19- | 31904 | 21 | 5697 | 6468 | 3613 | 5186 | 30 | 18 | 35 | 72.5 |
| CD828Z | | | | | | | | | | |
| Hu1928- | 16420 | 30713 | 6299 | 9285 | 8420 | 6634 | 23 | 24 | 27 | 58.6 |
| C2B8BB | | | | | | | | | | |
| Hu1928- | 6485 | 17241 | 1728 | 4788 | 4581 | 4010 | 12 | 11 | 17 | 62.7 |
| 11B8BB | | | | | | | | | | |
| C2B8- | 55 | 36499 | 800 | 6588 | 9074 | 7433 | 45 | 70 | 225 | 54.5 |
| CD828Z | | | | | | | | | | |

All values are IFNγ in pg/ml except the last column, which is the percentage of each culture that expressed the Indicated CAR by flow cytometry.
Nalm6, Toledo, ST486 all express both CD19 and CD20
NGFR-K562 and CCRF-CEM lack both CD19 and CD20

TABLE 9
Antigen-specific IFNy production after overnight co-culture
with target cells

| | | Target cells | | | | T- | |
|---|---|---|---|---|---|---|---|
| Effector | CD19- | CD20- | | NGFR- | | cells | % |
| T cells | K562 | K562 | ST486 | K562 | CEM | Alone | CAR+ |
| Un- | 45.2 | 40.2 | 439.6 | 46.2 | 9.3 | 9.2 | 0.0 |
| trans- | | | | | | | |
| duced | | | | | | | |
| Hu1928- | 60107.2 | 80247.4 | 22306.8 | 383.3 | 665.0 | 564.9 | 63.6 |
| 2.1.2BB | | | | | | | |
| Hu1928- | 51671.7 | 79912.1 | 24461.3 | 160.6 | 154.1 | 157.6 | 53.5 |
| 8G6-5BB | | | | | | | |
| Hu1928- | 57137.1 | 71880.0 | 27711.9 | 40.8 | 8.5 | 4.9 | 55.6 |
| GA101BB | | | | | | | |
| Hu1928- | 53172.8 | 78206.5 | 30321.8 | 92.1 | 35.1 | 47.4 | 48.6 |
| C2B8BB | | | | | | | |

All values are IFNγ in pg/mL except the last column, which is the percentage of each culture that expressed the Indicated CAR by flow cytometry.
CD19-K562 expresses CD19 and CD20-K562 expresses CD20
ST486 expresses both CD19 and CD20
NGFR-K562 and CCRF-CEM lack both CD19 and CD20

### Example 3

This example demonstrates that the first and second CARs encoded by the bicistronic constructs undergo CD19 and CD20 specific degranulation.

CAR T cells or untransduced T cells were assessed for CD107a upregulation, which is a marker of degranulation. The T cells transduced with the indicated bicistronic CAR constructs were cultured for 4 hours with either CD20-K562 cells, CD19-K562 cells, or the negative control NGFR-K562 cells.

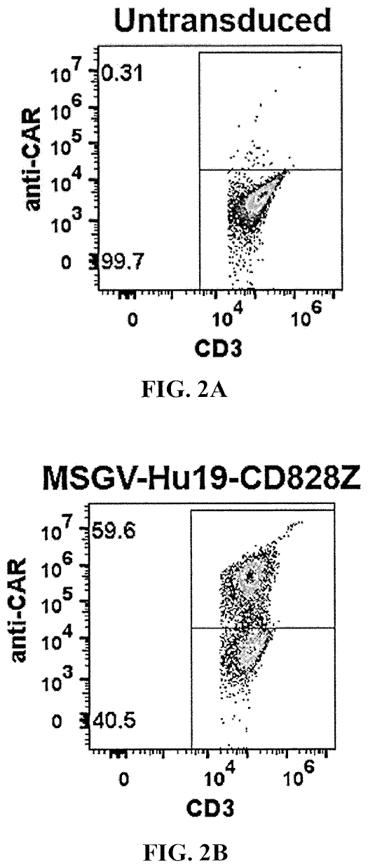

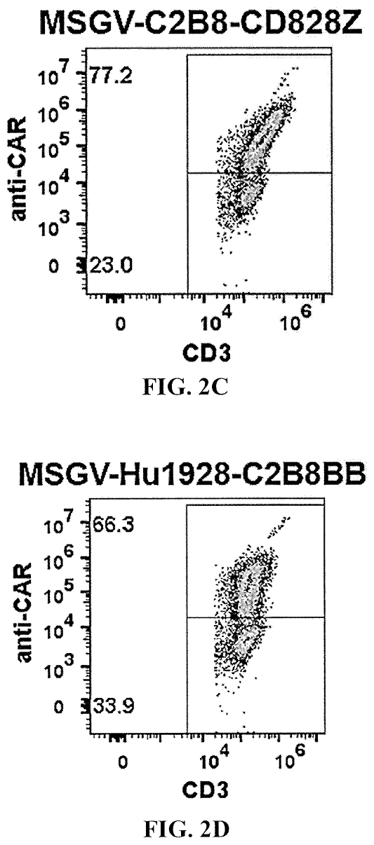

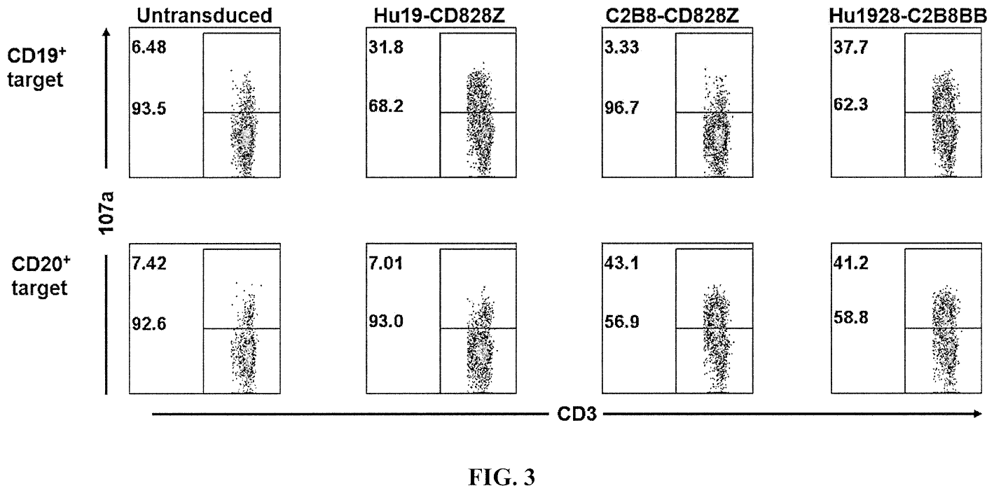

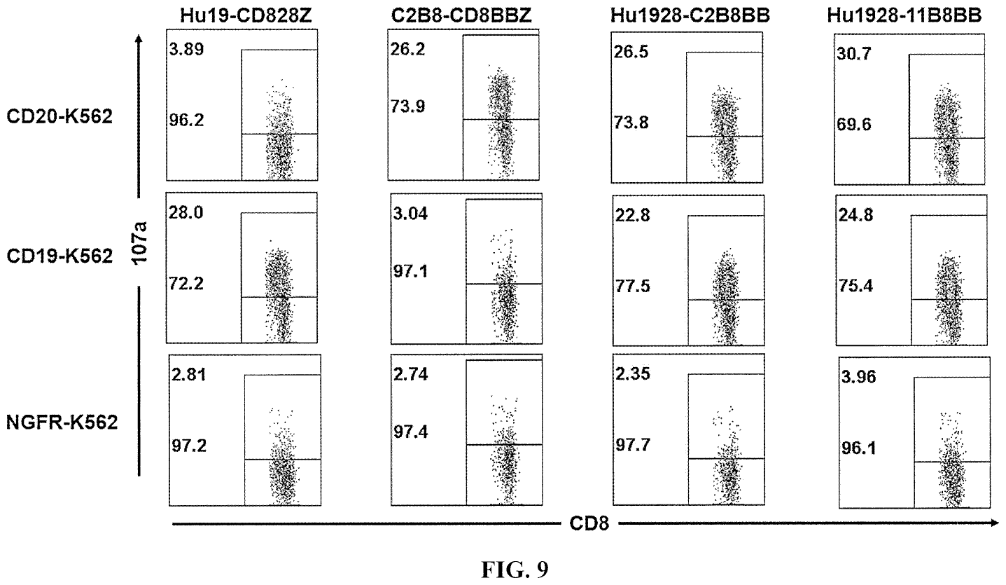

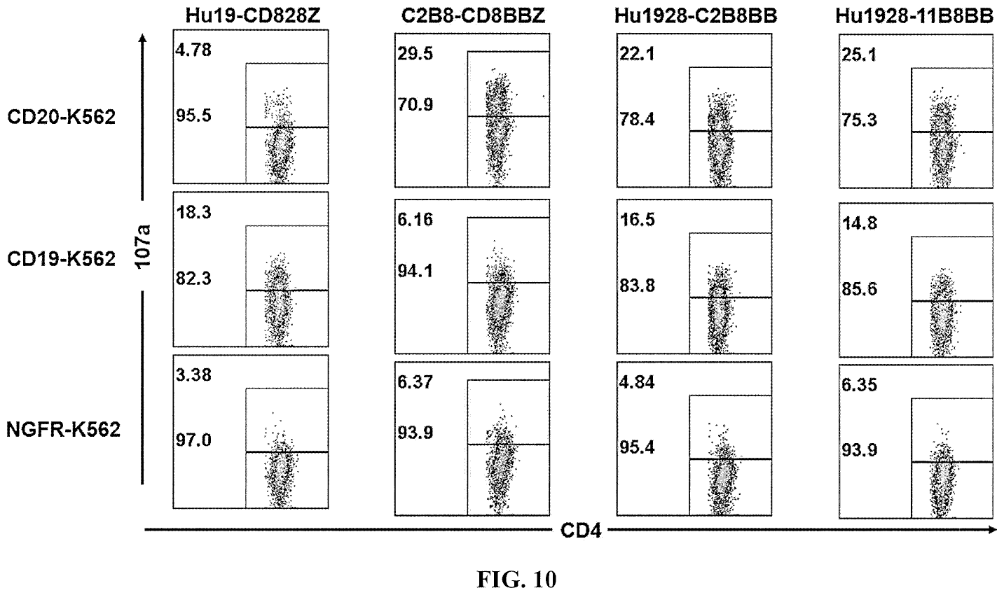

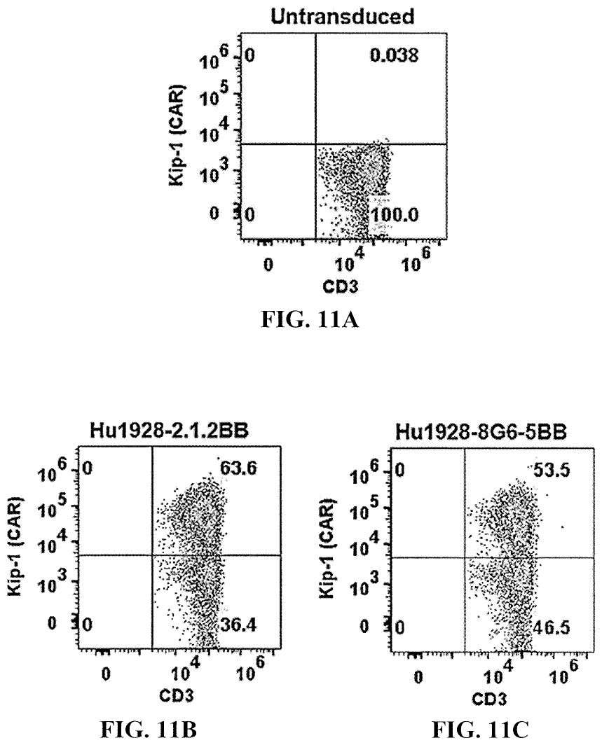

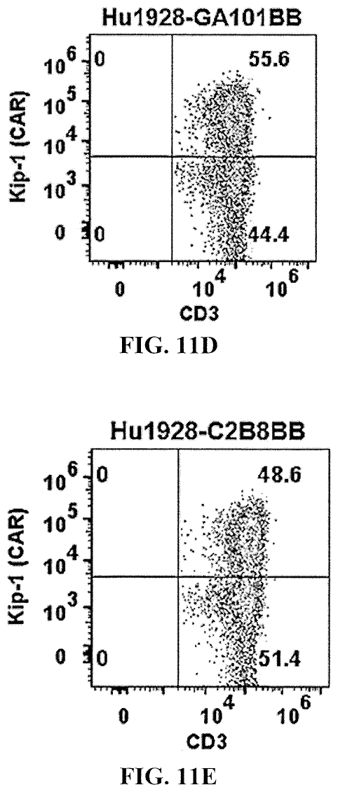

FIG. 3 shows that the CAR-expressing CD8⁺ T cells degranulate in an antigen-specific manner in response to Hu19-CD828Z, C2B8-CD828Z, or Hu1928-C21B811. FIG. 4 shows that the CAR-expressing CD4⁺ T cells degranulate in an antigen-specific manner in response to Hu19-CD828Z, C2B8-CD828Z, or Hu1928-C21B811. FIG. 5 shows that the CAR-expressing T cells specifically recognize CD19 and/or CD20. The Hu1928-C2B8BB-expressing T cells degranulated to a greater degree when co-cultured with either CD19 or CD20-expressing target cells. Further, the CD4⁺ CAR T cells (FIGS. 13A-B and 14A-B) and CD8+ CAR T cells (FIGS. 15A-B and 16A-B) degranulated in response to the CD19⁺ (FIGS. 13A-B and 15A-B) and CD20⁺ (FIGS. 14A-B and 16A-B) cells.

### Example 4

This example demonstrates that the T cells which express the anti-CD19/anti-CD20 bicistronic CAR constructs successfully kill lymphoma cells.

T cells were untransduced or were transduced with Hu19-CD828Z, C2B8-CD828Z, or Hu1928-C2B8BB. The T cells were co-cultured with cells of the CD19⁺, CD20⁺ lymphoma cell line Toledo and with CCRF-CEM negative control cells that lack CD19 and CD20 expression.

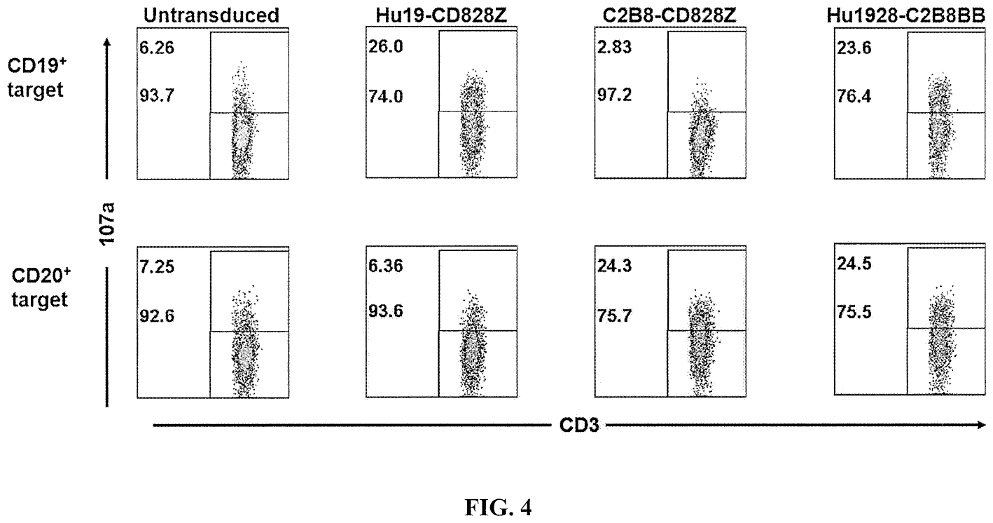

As shown in FIG. 6, Hu1928-C2B8BB-expressing T cells efficiently kill lymphoma cell line cells.

### Example 5

This example illustrates the cytotoxicity and proliferation of T cells expressing CD19/anti-CD20 bicistronic CAR constructs.

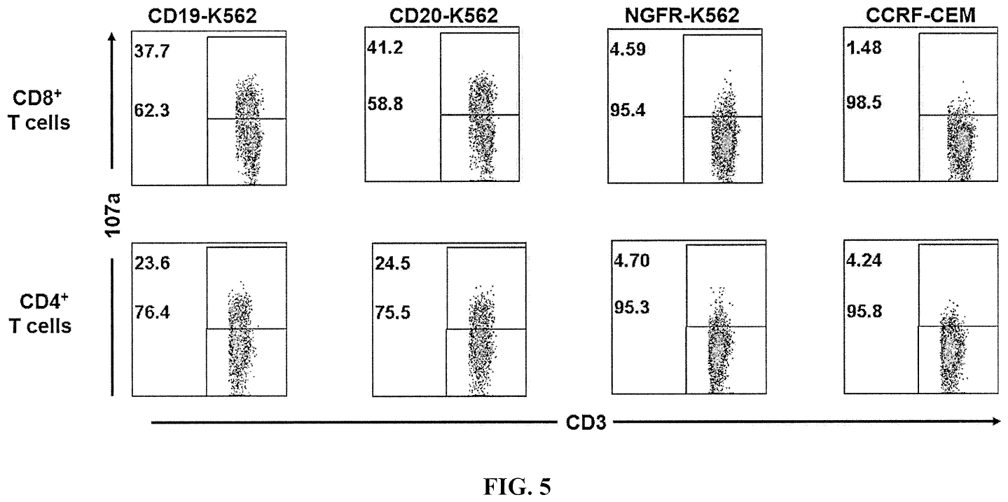

A cytotoxicity assessment of anti-CD19/anti-CD20 CAR construct-transduced T cells revealed that the CAR-expressing T cells proliferated preferentially when exposed to cells expressing their target antigen. As seen in FIGS. 7A-7D, the cell counts on the y-axis indicate that the number of T cells at the end of the culture period was higher when CAR T cells were exposed to target antigen(s). FIGS. 7A and 7B are graphs from cells that were transduced with Hu19-CD828Z, FIGS. 7C and 7D are graphs from cells that were transduced with Hu19-CD828Z, and FIGS. 7E and 7F are graphs from cells that were transduced with Hu1928-C2B8BB.

### Example 6

This example illustrates that the CD19/anti-CD20 bicistronic CAR constructs are expressed on primary human T cells.

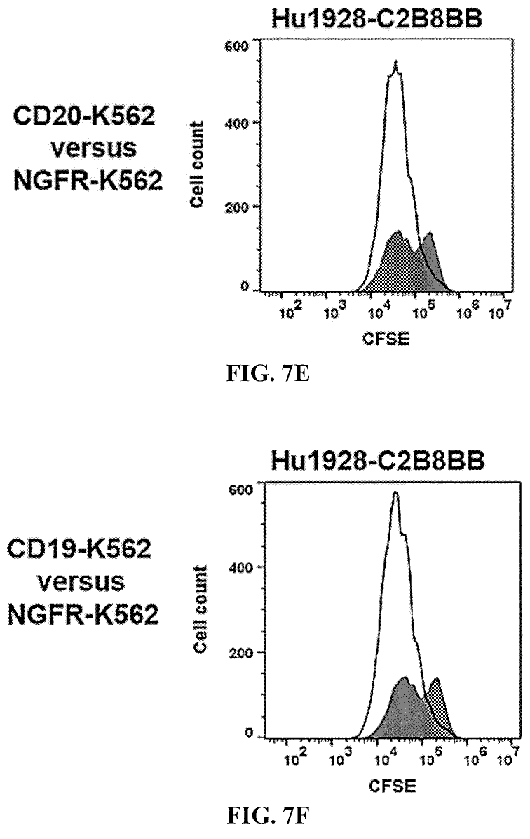

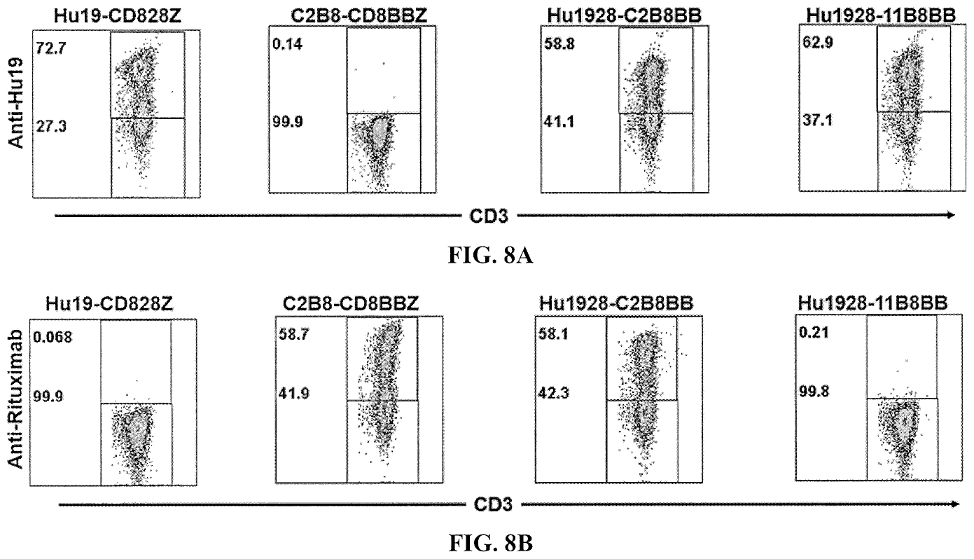

APC-labeled antibodies designated Kip-1 and Kip-4 were used to detect the CARs. FIG. 11B shows the plot from the cells that were transduced with Hu1928-2.1.2BB. FIG. 11C shows the plot from the cells that were transduced with Hu1928-8G6-5BB. FIG. 11D shows the plot from the cells that were transduced with Hu1928-GA101BB. FIG. 11E shows the plot from the cells that were transduced with Hu1928-C2B8BB. FIG. 12B shows the plot from when the expression of 2.1.2BB was evaluated. FIG. 12C shows the plot from when the expression of 8G6 was evaluated. FIG. 12D shows the plot from when GA101BB was evaluated. FIG. 12E shows the plot from when C2B8 was evaluated.

### Example 7

This example illustrates that the CD19/anti-CD20 bicistronic CAR constructs are effective at treating cancer.

ST486 (ATCC) tumors (B lymphocyte, Burkitt's lymphoma) were established in immunocompromised NOD scid gamma mice (NSG mice, The Jackson Laboratory). Four million tumor cells were allowed to grow for six days and then 4 million CAR T cells were injected into the mice.

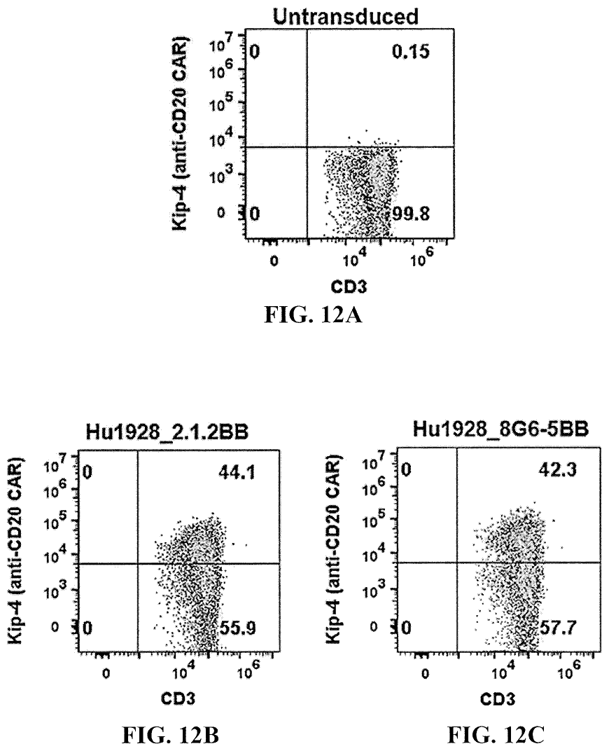

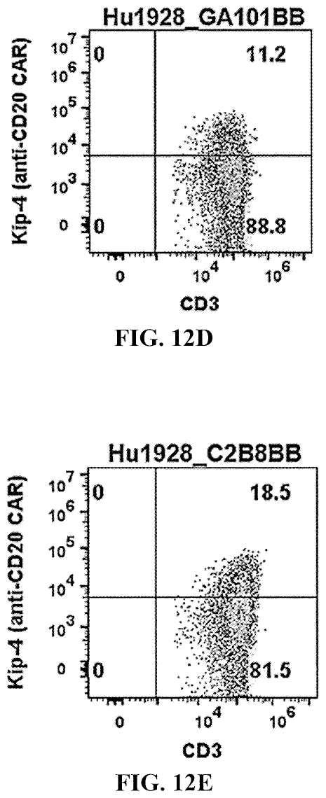

FIG. 17 shows that the constructs of the present invention eradicated tumors in mice. As seen in FIG. 17, the untransduced (open trianges) and SP6-CD828Z (open circles) transduced T cells allowed the tumors to increase in volume while the Hu1928-8G6-5BB (closed diamonds) and Hu1928-2.1.2BB (open squares) proved to be effective tumor treatments. FIG. 18 shows that treatment with the CARs of the present invention can increase survival rate of mice. As seen in FIG. 18, mice treated with untransduced (open trianges) and SP6-CD828Z (open circles) T cells showed zero percent survival in less than 30 days while the Hu1928-8G6-5BB (closed diamonds) and Hu1928-2.1.2BB (open squares) proved to be effective tumor treatments with 100 percent survival after 50 days.

### Example 8

This example illustrates that CD19/anti-CD20 bicistronic CAR constructs are expressed on the cell surface of T-cells after transduction.

T cells that were transduced with MSGV1-Hu1928-2.1.2BB were stained with 2 monoclonal antibodies. One of these antibodies, Kip-1 binds to the linker included in the Hu19 scFv of Hu19-CD828Z, and the other antibody, Kip-4, binds to the linker included in the Hu20 scFv of Hu20-CD8BBZ.

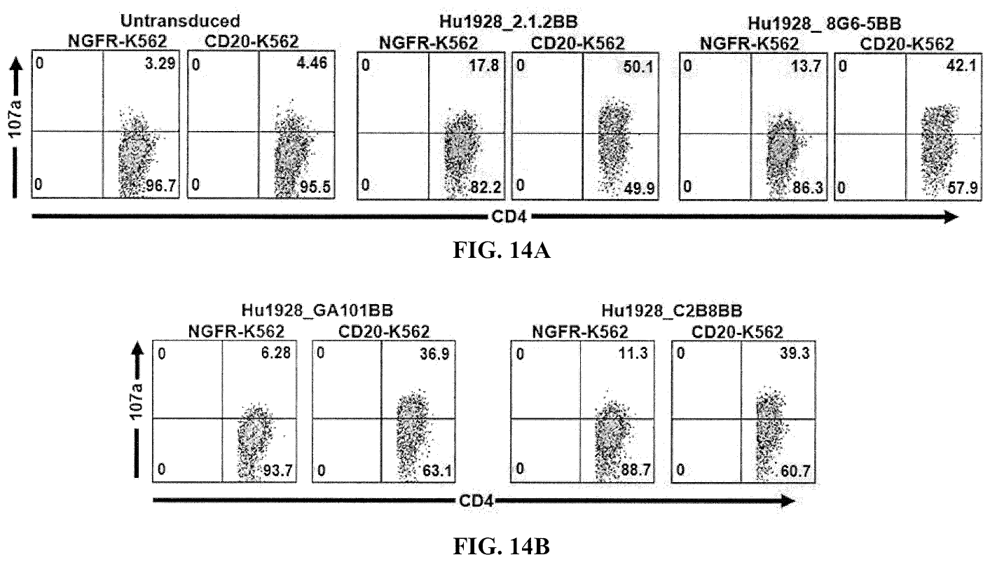

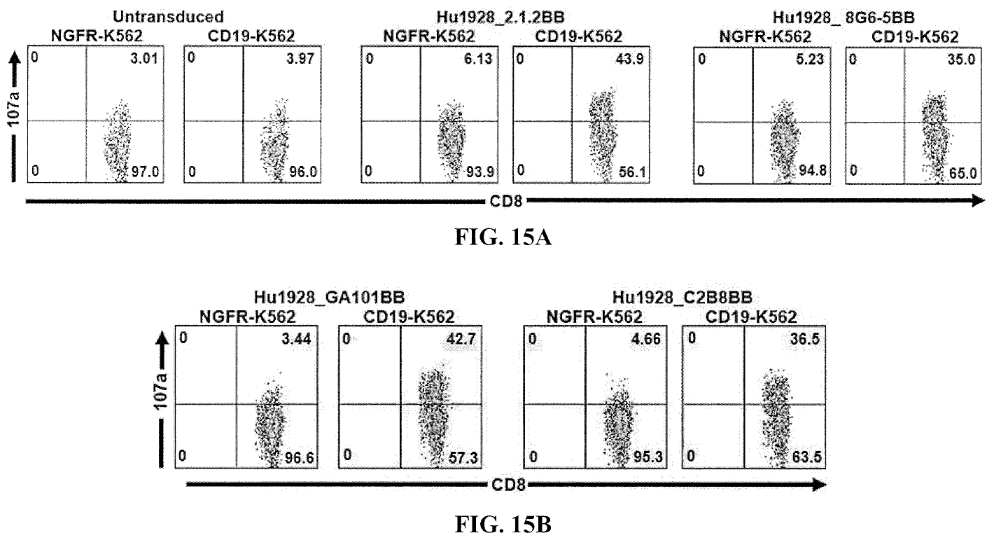

FIG. 20 shows expression of Hu19-CD828Z and Hu20-CD8BBZ on the surface of T cells five days after transduction. In this study, unselected PBMC were started in culture on day 0 by stimulating with an anti-CD3 monoclonal antibody in IL-2-containing medium. Transductions were carried out 2 days after the cultures were started, and the T cells were assessed for CAR expression 6 days later, when cells had been in culture for a total of 8 days. The plots in FIGS. 20 and 21 are gated on CD4⁺ or CD8⁺ live, CD3⁺ lymphocytes. FIG. 20 shows T cells stained with the Kip-1 antibody and FIG. 21 shows T cells stained with the Kip-4 antibody.

As seen in FIGS. 20 and 21, Hu19-CD828Z and Hu20-CD8BBZ are both present on the surface of T-cells after transduction with these CARs.

### Example 9

This example illustrates the CD20-binding specificity of CD19/anti-CD20 bicistronic CAR constructs.

HEK293 cells were transfected to express 5,647 human plasma membrane proteins. This allowed for screening against these human proteins for reactivity with antibody-based reagents (screening was performed by a third party, Retrogenix™). Untransduced human T cells and T cells from the same donor that expressed Hu20-CD8BBZ were used. The Hu20-CD8BBZ T cells were labeled and then used to screen the 5,647 human plasma membrane proteins.

The only differences in binding between the Hu20-CD8BBZ T cells and the untransduced T cells was for CD20, which was expected for this anti-CD20 CAR, and CD27. CD27 binding was very weak and inconsistent, but nonetheless, 293T cells were transduced and assessed for reactivity against Hu20-CD8BBZ T cells in an IFNγ ELISA.

No release above background was found when Hu20-CD8BBZ T cells were exposed to CD27⁺ target cells; therefore, it was determined that the Hu20-CD8BBZ CAR does not functionally recognize CD27. This study shows that the CD19/anti-CD20 bicistronic CAR constructs have desirably high specificity and are therefore unlikely to destroy normal tissues.

### Example 10

This example illustrates the specific degranulation of Hu1928-2.1.2BB-expressing T cells.

Degranulation of T cells is a prerequisite for perforin and granzyme-mediated cytotoxicity. Five tubes were prepared for each T-cell culture that was tested. The tubes contained target cells as follows: CD19 and CD20-negative NGFR-K562 cells, CD19⁺ CD19-K562 cells, CD20⁺ CD20-K562 cells, and ST486 cells that express CD20 and relatively low levels of CD19. All of the tubes contained CAR-transduced T cells, 1 ml of AIM-V medium+5% human AB serum, a titrated concentration of an anti-CD107a antibody (eBioscience, clone eBioH4A3), and 1 μL of GOLGISTOP™ (monesin, BD Biosciences). All of the tubes were incubated at 37° C. for 4 hours and then stained for CD3, CD4, and CD8

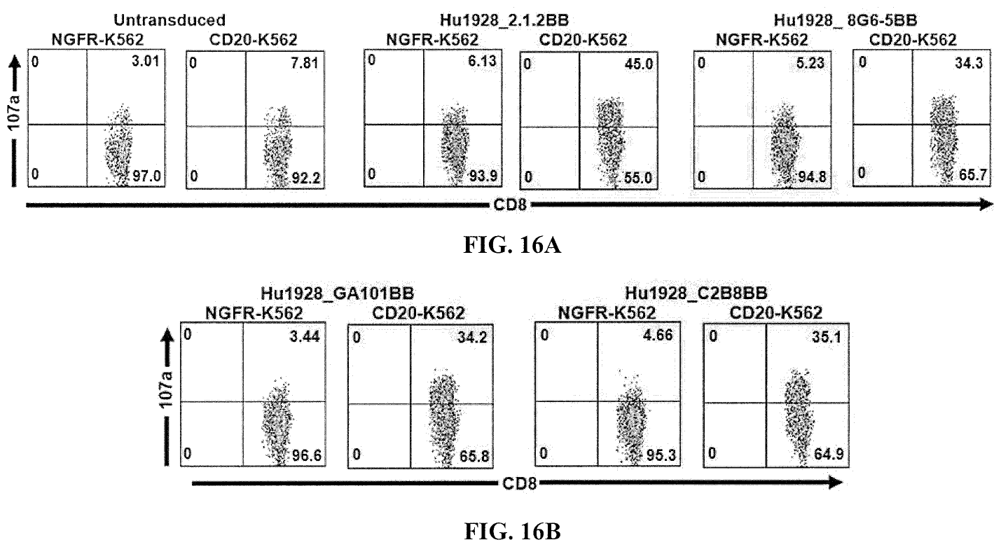

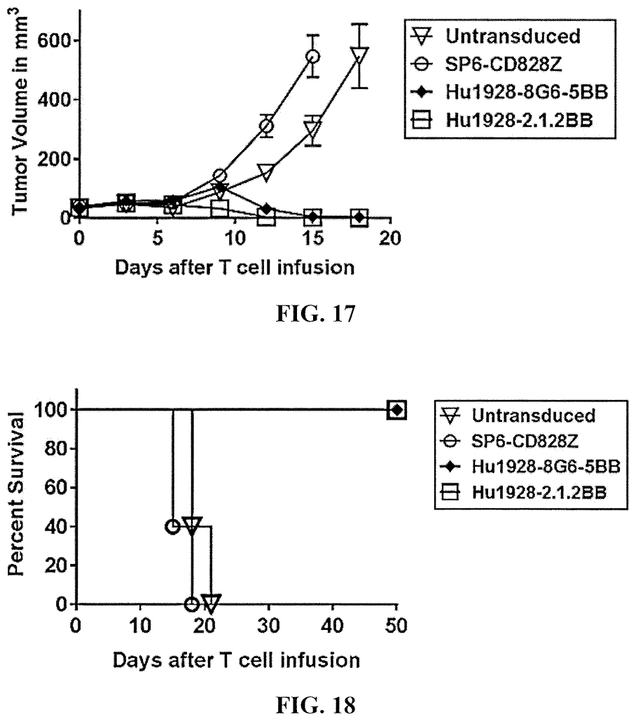

FIGS. 22 and 23 shows a representative CD107a assay in which untransduced (UT) T cells, Hu1928-2.1.2BB T cells, Hu19-CD828Z T cells (Hu1928), and Hu20-CD8BBZ T cells (2.1.2BB) were cultured for 4 hours with target cells. The T cells degranulated specifically in response to target cells with Hu1928-2.1.2BB T cells degranulating in response to CD19⁺ and/or CD20⁺ target cells, Hu19-CD828Z T cells degranulating in response to CD19⁺ target cells, and Hu20-CD8BBZ degranulating in response to CD20⁺ target cells. ST486 expresses low levels of CD19. FIG. 22 shows degranulation of CD8⁺ T cells and FIG. 23 shows degranulation of CD4⁺ T cells.

As explained above, this study shows that Hu1928-2.1.2BB-expressing T cells degranulate specifically in response to CD19⁺ and/or CD20⁺ target cells.

### Example 11

This example illustrates the in vitro proliferation of Hu1928-2.1.2BB-expressing T cells.

Cocultures were set up in 24-well plates. Target cells included in cocultures were either 0.5×10⁶ irradiated CD19-K562 cells, 0.5×10⁶ irradiated CD20-K562 cells, or 0.5×10⁶ irradiated NGFR-K562 cells. The cocultures also included 1×10⁶ T cells from cultures that had been transduced with either Hu1928-2.1.2BB or Hu19-CD828Z or Hu20-CD8BBZ. The T cells were labeled with CFSE. The medium used in the cocultures was AIM V+5% human AB serum. IL-2 was not added to the medium. Four days after initiation, the live cells in each coculture were counted by using trypan blue for dead cell exclusion, and flow cytometry was performed.

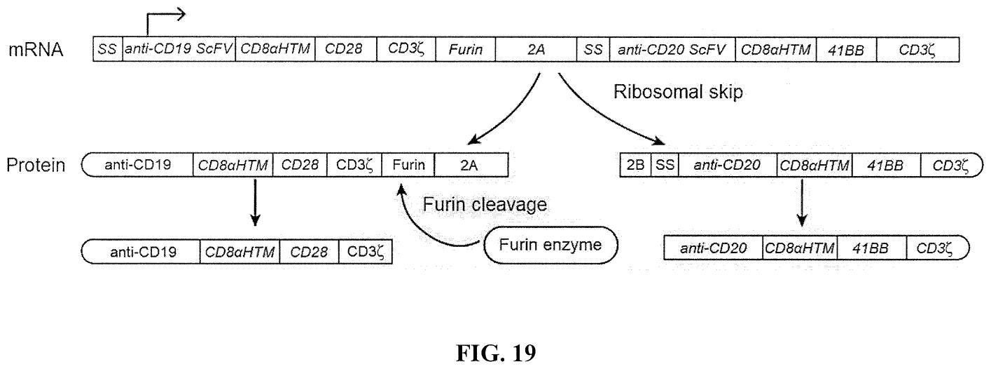

FIG. 24 shows results of this CFSE proliferation assay. The area under the curves of the histograms is proportionate to the number of cells. The histograms are labeled to indicate whether the T cells were stimulated with CD19-K562 cells, CD20-K562 cells, or NGFR-K562.

This study shows that T cells expressing Hu1928-2.1.2BB diluted CFSE, indicating proliferation, when cultured with either CD19⁺ target cells or CD20⁺ target cells. Although proliferation of Hu1928-2.1.2BB was greater when CD19⁺ or CD20⁺ target cells were present, there was some dilution of CFSE when the Hu1928-2.1.2BB-expressing T cells were cultured with NGFR-K562 cells, which lack expression of both CD19 and CD20. Hu19-CD828Z-expressing T cells diluted CFSE, indicating proliferation, only when cultured with CD19⁺ target cells. T cells expressing CARs with a CD28 moiety and no 4-1BB moiety were much more dependent on exposure to the relevant antigen for proliferation compared with CARs containing a 4-1BB moiety. Hu20-CD8BBZ T cells diluted CFSE to a greater extent when cultured with CD20⁺ target cells than when cultured with CD19⁺ target cells.

### Example 12

This example illustrates the cytotoxicity of T cells expressing anti-CD19/anti-CD20 bicistronic CAR constructs.

Cytotoxicity was measured by comparing the survival of CD19⁺ and CD20⁺ Toledo human lymphoma cell line target cells relative to the survival of negative-control CCRF-CEM target cells that do not express CD19 or CD20. Both target cell types were combined in the same tubes with CAR-transduced T cells. CCRF-CEM negative-control cells were labeled with the fluorescent dye 5-(and-6)-(((4-chloromethyl)benzoyl)amino) tetramethylrhodamine (CMTMR) (Invitrogen), and Toledo CD19⁺ and CD20⁺ target cells were labeled with CFSE. Cocultures were set up in sterile 5 mL test tubes in duplicate at multiple T cell to target cell ratios. The target cells contained in the tubes were 50,000 CD19⁺ and CD20⁺ Toledo target cells along with 50,000 CCRF-CEM negative-control cells. The cultures were incubated for 4 hours at 37° C. Immediately after the incubation, 7AAD (7-amino-actinomycin D) was added, and flow cytometry acquisition was performed. For each T cell plus target-cell culture, the percent survival of Toledo target cells was determined by dividing the percent live Toledo cells by the percent live CCRF-CEM negative-control cells. The corrected percent survival of Toledo target cells was calculated by dividing the percent survival of Toledo target cells in each T cell plus target cell culture by the ratio of the percent live Toledo target cells to percent live CCRF-CEM negative-control cells in tubes containing only Toledo target cells and CCRF-CEM cells without effector T cells. This correction was necessary to account for variation in the starting cell numbers and for spontaneous target cell death. Cytotoxicity was calculated as follows: the percent cytotoxicity of Toledo target cells=100-corrected percent survival of Toledo target cells. This method was used to compare the cytotoxicity of untransduced T cells (UT) and T cells expressing one of 3 different CARs: Hu1928-2.1.2BB, Hu19-CD828Z, and Hu20-CD8BBZ.

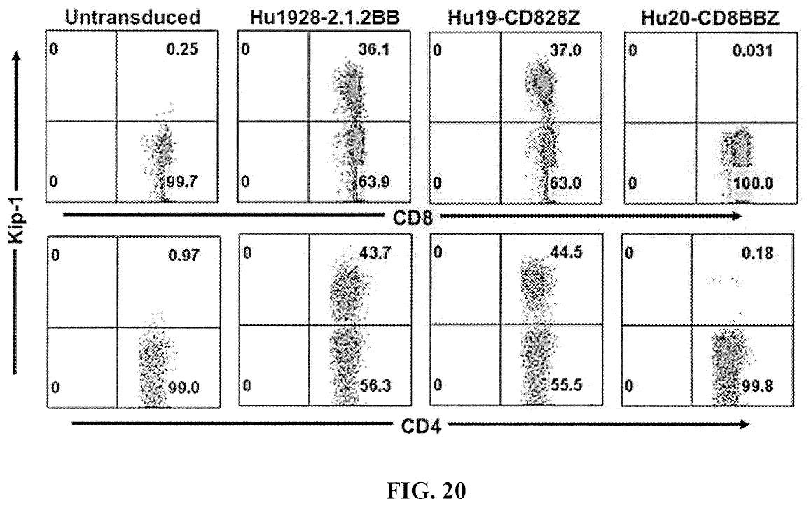

As seen in FIG. 25, T cells expressing Hu1928-2.1.2BB, Hu19-CD828Z, or Hu20-CD8BBZ killed human lymphoma cell line target cells expressing CD19 and CD20.

### Example 13

This example illustrates the in vitro CD20-binding specificity of anti-CD19/anti-CD20 bicistronic CAR constructs.

CAR-expressing T cells or untransduced T cells from the same patient were cultured with target cells overnight, and then a standard IFNγ enzyme-linked immunosorbent assay (ELISA) was performed. The T cells were then evaluated to see if they were activated, as indicated by IFNγ release, when the T cells were cultured with target cells (see Tables 10-12 below). The CAR T cells specifically reacted with target cells expressing CD19 and/or CD20, which is indicated by much higher levels of IFNγ release when the T cells are cultured with targets expressing CD19 and/or CD20 compared with when the T cells are cultured with target cells expressing neither CD19 nor CD20.

K562 cells were transduced to express CD19 (CD19-K562), low-affinity nerve growth factor (NFGR-K562), or CD20. All of these genes were transferred to K562 cells by standard methods with the MSGV gamma-retroviral vector. The NGFR-K562 cells served as CD19-negative control cells. NALM6 and ST486 cell lines were used as well as the following CD19-negative cell lines: melanoma cell line 624, the leukemia cell line NGFR-K562, the T-cell leukemia cell line CCRF-CEM; A549 (a lung carcinoma cell line); MDA-MB231 (a breast cancer cell line), Tc71 (a Ewings sarcoma cell line), COL0205 (a colon carcinoma cell line), U251 (a glioblastoma cell line), Panc10.05 (a pancreatic carcinoma cell line), HepG2 (hepatocellular carcinoma), and A431-H9 (an epidermoid (skin) carcinoma cell line that was transduced with the gene for mesothelin). Reactivity of CAR T cells with human primary cells was also assessed (Table 12). The following primary human cells were obtained from Lonza: renal proximal tubular epithelial cells, skeletal muscle cells, hepatic cells, renal cortical epithelial cells, and mammary epithelial cells. In each experiment, the result for effector T cell cultured alone was also given.

ELISA assays were performed on culture supernatant from overnight co-cultures of T cells plus target cells expressing CD19 and/or CD20 or target cells negative for both CD19 and CD20. In the data shown in Table 10, T cells from a patient were either left untransduced or transduced with genes encoding Hu1928-2.1.2BB, Hu19-CD828Z, or Hu20-CD8BBZ.

Table 11 shows IFNγ release by either Hu1928-2.1.2BB CAR T cells or untransduced T cells when these T cells were cultured overnight with CD19-K562, CD20-K562, or a panel of human cell lines that were negative for both CD19 and CD20.

Table 12 shows IFNγ release when a panel of primary human cells were cultured with T cells from a patient that were either left untransduced or transduced with genes encoding Hu1928-2.1.2BB, Hu19-CD828Z, or Hu20-CD8BBZ.

The percentage of T cells that expressed each CAR is listed on the extreme right column of each table below. This number was determined by staining the CAR-transduced T cells and the untransduced T cells with the Kip-1 antibody or the Kip-4 antibody. The cells were analyzed by flow cytometry, and the percentages of untransduced T cells that stained with the appropriate antibody was subtracted from the percentage of CAR-transduced T cells that stained with Kip-1 or Kip-4 to obtain the percent CAR⁺ T cells.

T cells transduced with anti-CD19 and/or anti-CD20 CARs produced large amounts of IFNγ when they were cultured overnight with cell lines expressing the appropriate target antigen. Hu1928-2.1.2BB T cells did not release IFNγ in response to cell lines that were negative for both CD19 and CD20 (see Tables 10-12). All cytokine values in Tables 10-12 are IFNγ levels in picograms/mL.

One potential problem with the use of anti-CD20 CARs is possible blocking by serum anti-CD20 antibodies that a patient may have previously received. Anti-CD20 monoclonal antibodies, such as rituximab, might block binding of CAR T cells to lymphoma cells. Prior reports have assessed rituximab levels in the serum of patients and found that the median serum rituximab concentration to be 38.3 μg/mL (Rufener, et al., Cancer Immunology Research, 4: 509-519 (2016)) in patients who had received rituximab in the past 4 months.

In view of this, the impact of soluble rituximab on anti-CD20 CAR T cells was accessed by performing ELISA assays in which Hu1928-2.1.2BB CAR T cells, CD20⁺ target cells, and graded concentrations of rituximab were added together in overnight cultures. After the cultures, IFNγ ELISAs were performed on the culture supernatant. Rituximab did decrease IFNγ release in a dose-dependent manner, but it never eliminated the ability of the CAR T cells to recognize lymphoma (see Table 13). All cytokine values in Table 13 are μg/mL. The rituximab concentrations are at the top of the table. Target cells used were ST486−/− cells that express CD20 but not CD19. Human IgG was added to some wells as a control.

TABLE 10

| | | | | | | | | T- | % |
|---|---|---|---|---|---|---|---|---|---|
| | CD19- | CD20- | | | ST486- | NGFR- | CCRF- | cells | CAR |
| | K562 | K562 | NALM6 | ST486 | CD19-/- | K562 | CEM | Alone | + |
| Untransduced | 441 | 368 | 18 | 390 | 197 | 530 | 12 | 7 | 0 |
| Hu1928- | 10103 | 21162 | 6685 | 7970 | 8317 | 781 | 88 | 52 | 44 |
| 2.1.2BB | | | | | | | | | |
| Hu19- | 13280 | 808 | 6524 | 1433 | 641 | 918 | 39 | 20 | 36 |
| CD828Z | | | | | | | | | |
| Hu20- | 171 | 4014 | 1647 | 4620 | 4506 | 115 | 137 | 128 | 48 |
| CD8BBZ | | | | | | | | | |

All values are IFNγ in μg/ml except the last column, which is the percentage of each culture that expressed the Indicated CAR by flow cytometry.

T cells were cultured with the indicated target cells overnight, and an IFNγ ELISA was performed.

CD19-K562 expresses CD19 and CD20-K562 expresses CD20; all other targets listed lack both CD19 and CD20.

TABLE 11

| | | | | | | | | | | | | T- | |
|---|---|---|---|---|---|---|---|---|---|---|---|---|---|
| | K562- | K562- | A431- | Panc- | | Colo- | Hep- | MDA- | | | | cells | % CAR |
| T cells | CD19 | CD20 | H9 | 10.05 | U251 | 205 | G2 | MB231 | A549 | Tc71 | 624 | Alone | + |
| Un- | <12 | <12 | <12 | <12 | 76 | <12 | <12 | 873 | 66 | 15 | <12 | <12 | 0.0 |
| trans- | | | | | | | | | | | | | |
| duced | | | | | | | | | | | | | |
| Hu1928- | 9343 | 6127 | 79 | 88 | 124 | 27 | 90 | 459 | 75 | 97 | 104 | 74 | 51 |
| 2.1.2BB | | | | | | | | | | | | | |

TABLE 12

| | | | | Proximal | | | Renal | Mammary | T- | % |
|---|---|---|---|---|---|---|---|---|---|---|
| | CD19- | CD20- | NGFR- | tubular | Skeletal | Hepatic | cortical | epith. | cells | CAR |
| T cells | K562 | K562 | K562 | cells | muscle | cells | epith. | Cells | Alone | + |
| Un- | 56 | 31 | 40 | 54 | 15 | 36 | 108 | <12 | <12 | 0.0 |
| trans- | | | | | | | | | | |
| duced | | | | | | | | | | |
| Hu1928- | 5536 | 10929 | 109 | 128 | 79 | 35 | 174 | 144 | 106 | 91.1 |
| 2.1.2BB | | | | | | | | | | |
| Hu19- | 13946 | 118 | 85 | 38 | 27 | 20 | 107 | 19 | 15 | 74.4 |
| CD828Z | | | | | | | | | | |
| Hu20- | 255 | 10197 | 280 | 268 | 146 | 48 | 374 | 251 | 224 | 86.0 |
| CD8BBZ | | | | | | | | | | |

All values are IFNγ in μg/ml except the last column, which is the percentage of each culture that expressed the Indicated CAR by flow cytometry.

T cells were cultured with the indicated primary human target cells overnight, and an IFNγ ELISA was performed.

CD19-K562 expresses CD19 and CD20-K562 expresses CD20; all other targets listed lack both CD19 and CD20.

TABLE 13

| Average pg/mL | 100 | 50 | 25 | 12.5 | 6.2 | 0 | |
|---|---|---|---|---|---|---|---|
| IFNg | ug/ml | ug/ml | ug/ml | ug/ml | ug/ml | ug/ml | % CAR+ |
| Human IgG | 5921 | 6514 | 5470 | 5591 | 5841 | 6490 | 57.3 |
| Rituximab | 1661 | 2925 | 4165 | 5309 | 6165 | 6388 | 57.3 |

T cells transduced with the Hu1928-2.1.2BB produced large amounts of IFNγ when they were cultured overnight with cell lines expressing either CD19 or CD20 but only small amounts of IFNγ when cultured with human cell lines or primary human cells that lacked expression of both CD19 and CD20. The results show that Hu1928-2.1.2BB CART cells specifically recognized target cells expressing either CD19 or CD20 or both CD19 and CD20. Hu1928-2.1.2BB T cells did not specifically recognize any of a variety of cell lines and primary cells that lacked CD19 and CD20 expression. In addition, the constituent CARs of the Hu1928-2.1.2BB construct are the anti-CD19 CAR Hu19-CD828Z and the anti-CD20 CAR Hu20-CD8BBZ. Hu19-CD828Z specifically recognized CD19⁺ targets while Hu20-CD8BBZ specifically recognized CD20⁺ targets.

Presence of rituximab in culture media along with CAR T cells and target cells expressing CD20 did partially block IFNγ release from Hu1928-2.1.2BB T cells, but a substantial amount of IFNγ was released at all rituximab concentrations including concentrations equal to the concentrations of rituximab found in patient blood after patients received rituximab clinically. These results from this one study possibly imply that rituximab can partially block recognition of lymphoma cells by Hu1928-2.1.2BB T cells.

### Example 14

This example illustrates that anti-CD19/anti-CD20 bicistronic CAR constructs kill primary leukemia cells in vitro.

T cells left untransduced or transduced with Hu1928-2.1.2BB or transduced with the negative-control CAR SP6-CD828Z were assessed in a cytotoxicity assay as described in Example 12 above except that primary human chronic lymphocytic leukemia cells were used as the CD19⁺ and CD20⁺ target cells.

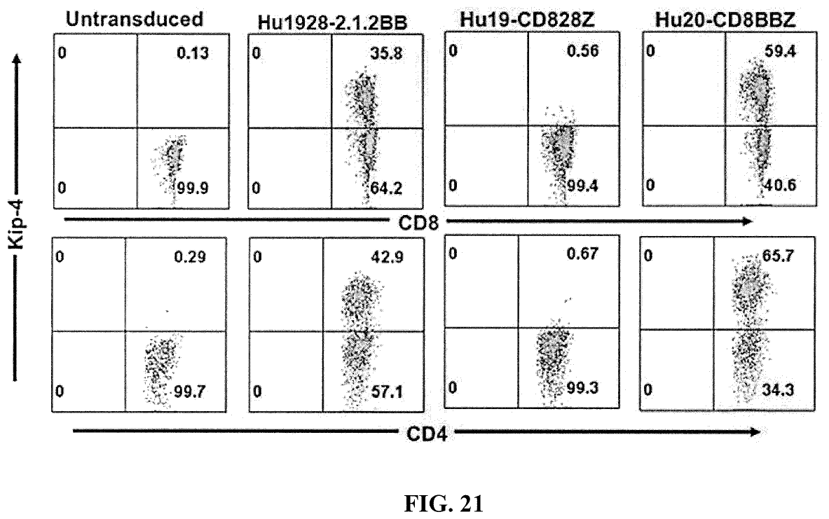

FIG. 26 shows that T cells expressing Hu1928-2.1.2BB could specifically kill primary chronic lymphocytic leukemia cells.

### Example 15

This example illustrates that anti-CD19/anti-CD20 bicistronic CAR constructs are effective at eradicating tumor.

This study evaluated the in vivo anti-tumor efficacy and toxicity of human T cells expressing Hu1928-2.1.2BB and the dose-response curves of Hu1928-2.1.2BB-expressing CAR T cells in mice.

Immunocompromised Nod-Scid common γ-chain knockout (NSG, NOD.Cg-Prkdc^{scid} Il2rg^{tm1Wjl}/SzJ) from The Jackson Laboratory mice were used. There were 5 mice in all experimental groups. In all mouse experiments, mice received only 1 infusion of CAR T cells and no other interventions. After CAR T-cell infusion, tumors were measured with calipers every 3 days. The longest length and the length perpendicular to the longest length and the tumor thickness were multiplied together and then divided by 2 to obtain the tumor volume in mm³. When the longest length reached 15 mm, the mice were sacrificed.

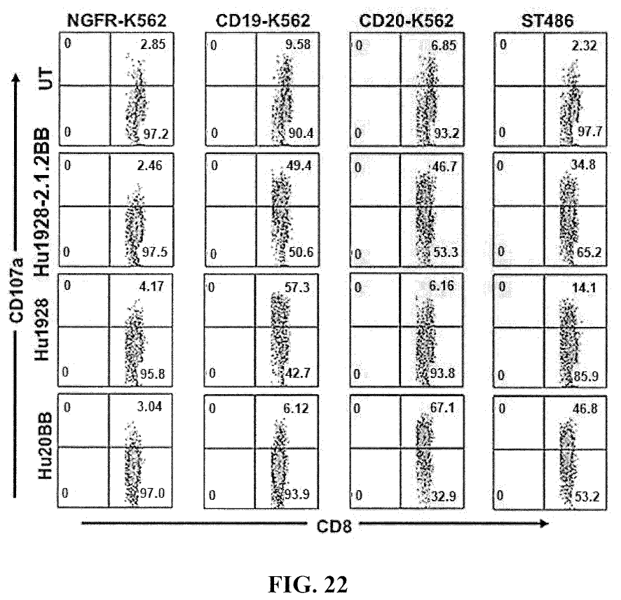

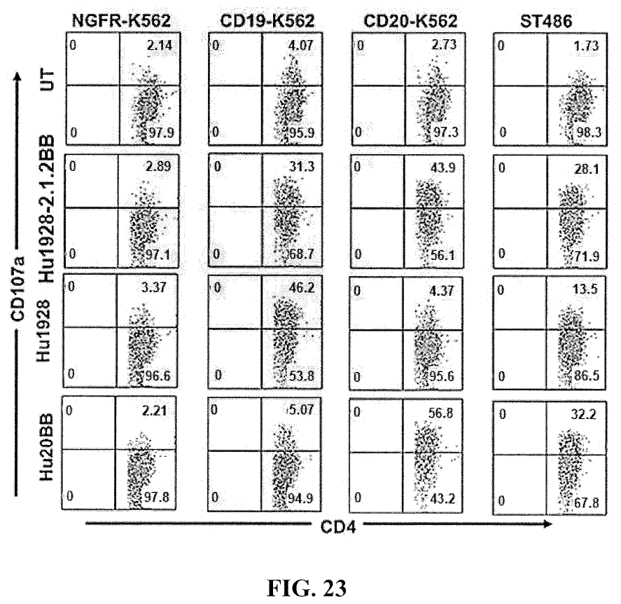

Results from a dose-titration experiment are shown in FIGS. 27 and 28. In this study, 4 million ST486 cells were injected 6 days to establish palpable intradermal tumors prior to CAR T cell infusion. Mice were then treated with a single infusion of graded doses of Hu1928-2.1.2BB T cells as shown in FIGS. 27 and 28. Tumor eradication was dose-dependent, and doses of 2 and 4 million CAR T cells had clear anti-tumor activity.

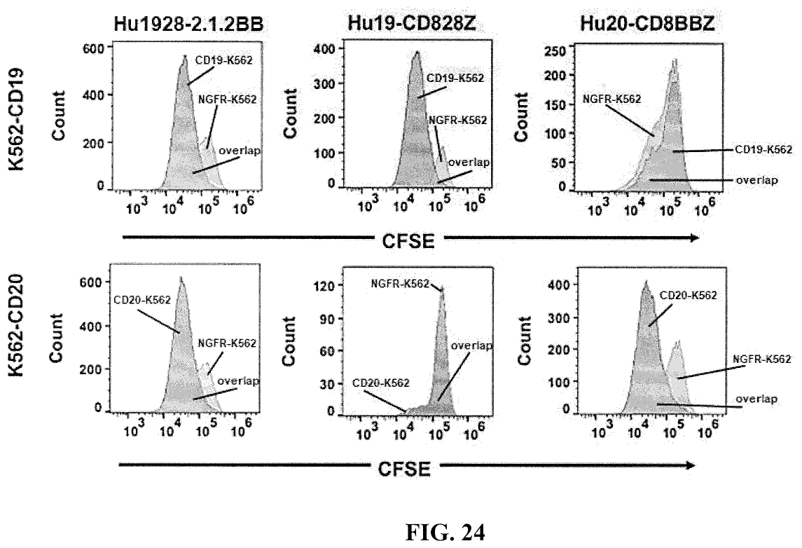

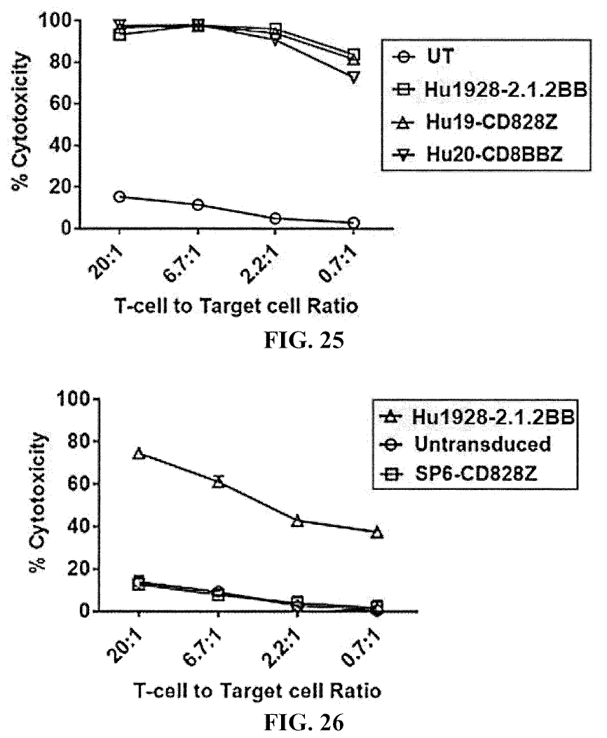

The anti-tumor activity of T cells expressing Hu1928-2.1.2BB and its constituent CARs was compared to the ST486 null (CD19−/−, CD19 expression was abrogated by CRISPR/Cas9). Four million ST486 (CD19-/−) cells were injected 6 days prior to CAR T-cell infusion to establish palpable intradermal tumors prior to CAR T-cell infusion. In this model, Hu1928-2.1.2BB and Hu20-CD8BBZ were much more effective than Hu19-CD828Z, which was expected because ST486 (CD19−/−) expresses CD20, but has very low levels of CD19 expression. The modest anti-tumor activity of Hu19-CD828Z may have been caused by Hu19-CD828Z T cells reacting against some residual CD19 that was expressed on the ST486 (CD19-/−) cells despite the attempt at CD19 abrogation. Results from this study are shown in FIGS. 29 and 30.

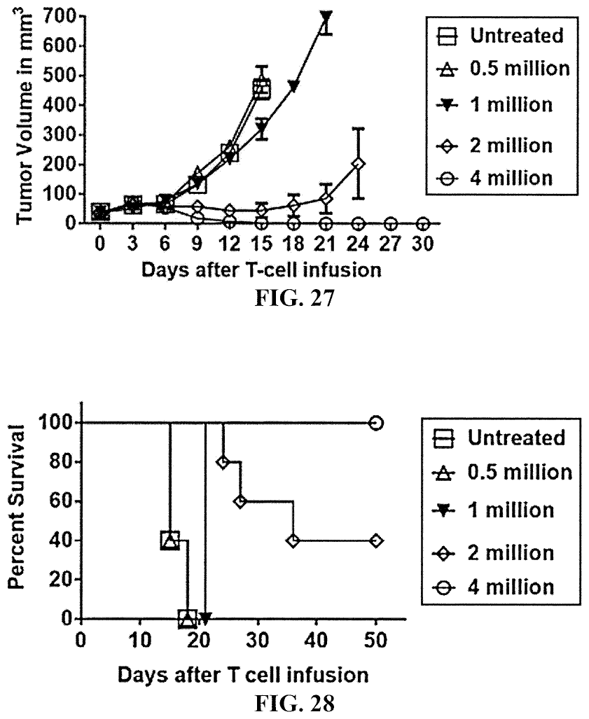

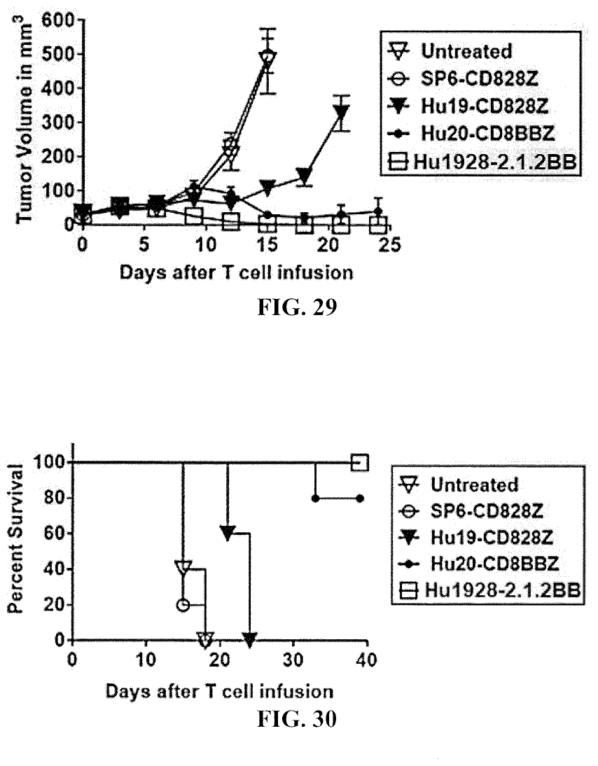

Hu1928-2.1.2BB CAR T cells were also tested against tumors of the NALM6 cell line NALM6 is CD19⁺ but CD20-negative. Four million NALM6 cells were injected intradermally into NSG mice to establish tumors. After 6 days, when palpable tumors were established, one group of mice was left untreated, and the other 3 groups were injected with 6 million CART cells. The T cells expressed either Hu1928-2.1.2BB, Hu19-CD828Z, or Hu20-CD8BBZ. Hu1928-2.1.2BB T cells eliminated the tumors in 5 of 5 mice, and Hu19-CD828Z-expressing T cells eliminated tumors in 4 of 5 mice with one mouse dying of a progressive tumor. In contrast, all of the Hu20-BBZ treated and untreated mice died. The lack of effectiveness of Hu20-CD8BBZ was expected due to the lack of CD20 expression on NALM6 cells. Results from this study are shown in FIGS. 31 and 32.

None of the mice receiving Hu1928-2.1.2BB T cells in these experiments exhibited signs of toxicity. The mice did not exhibit ruffled fur or decreased activity, and the mice died only when sacrificed at the end of the experiments or when sacrificed after large tumors developed.

These studies show that Hu1928-2.1.2BB-expressing T cells have dose-dependent activity against established tumors of human tumor cell lines. Hu1928-2.1.2BB-expressing T cells had strong anti-tumor activity against cells that lacked expression of either CD19 or CD20. Mice did not experience any signs of toxicity after the CAR⁺ T-cell infusions.

### Example 16

This example illustrates that anti-CD19/anti-CD20 bicistronic CAR constructs are non-toxic.

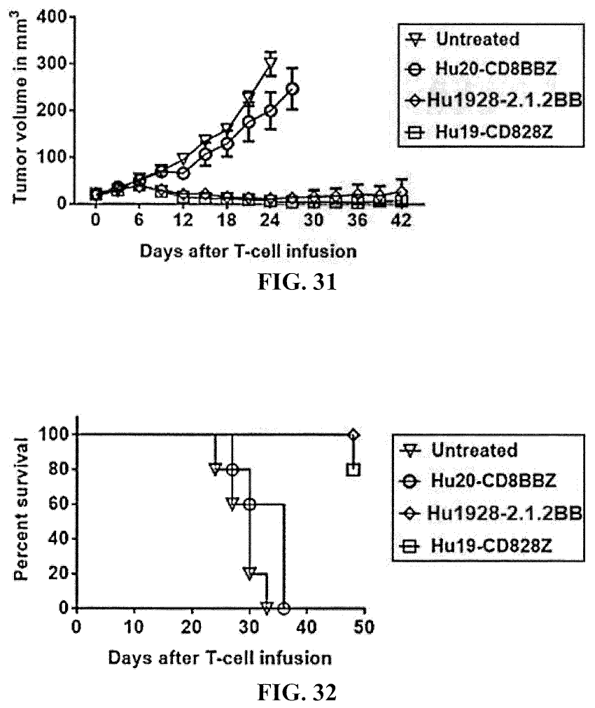

ST486 solid tumors were established in NSG mice, and then the mice were infused with untransduced T cells or 5×10⁶ CAR⁺ T cells. The T cells expressed either Hu1928-2.1.2BB, Hu20-CD8BBZ, or Hu19-CD828Z. The weight and serum interferon gamma (IFN-γ) of the mice (5 mice per group) were measured. The mean weight of the mice slightly increased during the period of measurement (see FIG. 33) and serum IFN-γ levels (see Table 14) in mice receiving Hu1928-2.1.2BB T cells were very similar to that of untreated mice.

This study did not provide any evidence of toxicity or high levels of IFN-γ in mice with solid tumors of ST486 cells when these mice were treated with Hu1928-2.1.2BB.

TABLE 14

| | Average IFN-gamma |
|---|---|
| Hu1928-2.1.2BB | 27 |
| Hu20-CD8BBZ | 43 |
| Hu19-CD828Z | 66 |
| Untransduced | 30 |

### Example 17

This example illustrates that anti-CD19/anti-CD20 bicistronic CAR constructs do not cause T cell immortalization.

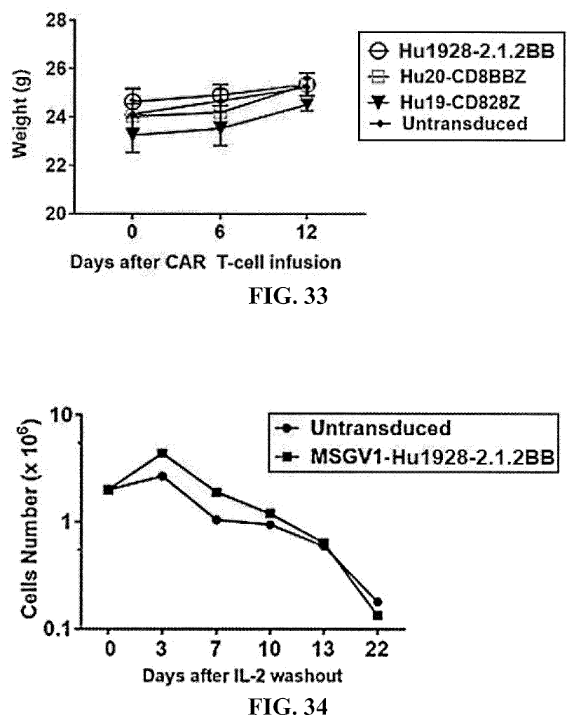

T cells transduced with MSGV1-Hu1928-2.1.2BB were observed in culture without exogenous IL-2. The study was performed on samples from 2 patients. Data from a representative sample is shown in FIG. 34. FIG. 34 shows that the transduced T cells were not immortalized because their numbers decreased steadily after IL-2 was washed out of the culture on day 0.

### Example 18

This example illustrates that anti-CD19/anti-CD20 bicistronic CAR constructs can be administered in combination with chemotherapy.

In this study, cyclophosphamide 500 mg/m² and fludarabine 30 mg/m² can be administered to a patient for 3 consecutive days. CAR T cells can be infused 3 days (approximately 72 hours) after the last dose of chemotherapy.

The administration of the conditioning chemotherapy regimen will allow for observation of enhanced effects of Hu1928-2.1.2BB-expressing T cells following the conditioning regimen. Administering chemotherapy or radiotherapy may enhance adoptive T-cell therapy with the anti-CD19/anti-CD20 bicistronic CAR constructs by multiple mechanisms including depletion of regulatory T cells and elevation of T-cell stimulating serum cytokines including interleukin-15 (IL-15) and interleukin-7 (IL-7), and possibly depletion of myeloid suppressor cells and other mechanisms. Removal of endogenous “cytokine sinks” by depleting endogenous T cells and natural killer cells caused serum levels of important T-cell stimulating cytokines such as IL-15 and IL-7 to increase, and increases in T-cell function and anti-tumor activity were dependent on IL-15 and IL-7 (see, e.g., Gattinoni, et al., Journal of Experimental Medicine, 202: 907-912 (2005)). Experiments in a murine xenograft model showed that regulatory T cells could impair the anti-tumor efficacy of anti-CD19 CAR T cells (Lee, et al., Cancer Research, 71: 2871-2881 (2011)). Myeloid suppressor cells have been shown to inhibit anti-tumor responses (Dumitru, et al., Cancer Immunology, 61: 1155-1167 (2012)). Experiments with a syngeneic murine model showed that lymphocyte-depleting total body irradiation (TBI) administered prior to infusions of anti-CD19-CAR-transduced T cells was required for the T cells to cure lymphoma. In these experiments, some mice received TBI, and other mice did not receive TBI. All mice were then challenged with lymphoma and treated with syngeneic anti-CD19-CAR T cells. Mice receiving TBI had a 100% cure rate, and mice not receiving TBI had a 0% cure rate (see Kochenderfer, et al., Blood, 116: 3875-3886 (2010).

Previous studies have provided strong suggestive evidence of enhancement of the activity of adoptively-transferred T cells in humans. Very few clinical responses have occurred and very little evidence of in vivo activity has been generated in clinical trials of anti-CD19-CAR T cells administered without lymphocyte-depleting chemotherapy. In contrast, many durable remissions of lymphoma and evidence of long-term B-cell depletion have occurred in clinical trials in which patients received anti-CD19-CAR T cells after lymphocyte-depleting chemotherapy. The chemotherapy regimen that best increases the anti-malignancy efficacy of CAR-expressing T cells is not known, but the chemotherapy regimens that have most convincingly been associated with persistence and in vivo activity of adoptively transferred T cells have included cyclophosphamide and fludarabine. Both cyclophosphamide and fludarabine are highly effective at depleting lymphocytes. One well-characterized and commonly used regimen is the combination of 300-500 mg/m² of cyclophosphamide administered daily for 3 days and fludarabine 30 mg/m² administered daily for three days on the same days as the cyclophosphamide. Multiple cycles of this regimen can be tolerated by heavily pretreated leukemia patients.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A nucleic acid comprising a nucleotide sequence encoding a chimeric antigen receptor (CAR) construct comprising:
(a) a first CAR comprising
a first antigen binding domain,
a first transmembrane domain, and
a first intracellular T cell signaling domain;
(b) a second CAR comprising
a second antigen binding domain,
a second transmembrane domain, and
a second intracellular T cell signaling domain; and
(c) cleavage sequence;
wherein the cleavage sequence is positioned between the first and second CARs,
wherein the first antigen binding domain of the first CAR has antigenic specificity for CD19, and
wherein the second antigen binding domain of the second CAR has antigenic specificity for CD20.

2. The nucleic acid according to claim 1, wherein the cleavage sequence comprises any one of the following: porcine teschovirus-1 2A (P2A) amino acid sequence, equine rhinitis A virus (E2A) amino acid sequence, thosea asigna virus 2A (T2A) amino acid sequence, foot-and-mouth disease virus (F2A) amino acid sequence, or a furin-cleavable amino acid sequence, modified versions of any of the foregoing, or any combination of the foregoing.

3. The nucleic acid according to claim 1, wherein the cleavage sequence comprises a foot-and-mouth disease virus (F2A) amino acid sequence.

4. The nucleic acid according to claim 1, wherein the cleavage sequence comprises an amino acid sequence comprising SEQ ID NO: 10.

5. The nucleic acid according to claim 1, wherein the first antigen binding domain comprises the six CDRs of Hu19.

6. The nucleic acid according to claim 1, wherein the first antigen binding domain comprises a first variable region comprising the amino acid sequence of SEQ ID NO: 4 and a second variable region comprising the amino acid sequence of SEQ ID NO: 6.

7. The nucleic acid according to claim 1, wherein the first antigen binding domain comprises single-chain variable fragment Hu19.

8. The nucleic acid according to claim 1, wherein the second antigen binding domain comprises the six CDRs of 11B8, C2B8, 2.1.2, 8G6, or GA101.

9. The nucleic acid according to claim 1, wherein the second antigen binding domain comprises an antigen binding domain of antibody C2B, 11B8, 8G6, 2.1.2, or GA101.

10. The nucleic acid according to claim 1, wherein one or both of the first and second transmembrane domain(s) comprises a CD8 transmembrane domain.

11. The nucleic acid according to claim 1, wherein one or both of the first and second CARs comprises a hinge domain.

12. The nucleic acid according to claim 1, wherein one or both of the first and second intracellular T cell signaling domain(s) comprises any one of the following: a human CD28 protein, a human CD3-zeta protein, a human FcRγ protein, a CD27 protein, an OX40 protein, a human 4-1BB protein, a human inducible T-cell costimulatory protein (ICOS), modified versions of any of the foregoing, or any combination of the foregoing.

13. The nucleic acid according to claim 1, wherein one or both of the first and second intracellular T cell signaling domain(s) comprises a CD28 intracellular T cell signaling sequence.

14. The nucleic acid according to claim 13, wherein the CD28 intracellular T cell signaling sequence comprises the amino acid sequence of SEQ ID NO: 8.

15. The nucleic acid according to claim 1, wherein one or both of the first and second intracellular T cell signaling domain(s) comprises a CD3 zeta (ξ) intracellular T cell signaling sequence.

16. The nucleic acid according to claim 15, wherein the CD3ξ intracellular T cell signaling sequence comprises the amino acid sequence of SEQ ID NO: 9.

17. The nucleic acid according to claim 1, wherein the CAR construct comprises a CD8 leader domain.

18. The nucleic acid according to claim 17, wherein the CD8 leader domain sequence comprises the amino acid sequence of SEQ ID NO: 3.

19. The nucleic acid according to claim 1, wherein the CAR construct comprises exactly two CARs being the first and second CARs, respectively.

20. The nucleic acid of claim 1, which encodes a CAR construct comprising the amino acid sequence of any one of SEQ ID NOs: 2, 16, 20, 24, or 29.

21. One or more polypeptide(s) encoded by the nucleic acid of claim 1.

22. A recombinant expression vector comprising the nucleic acid of claim 1.

23. An isolated host cell comprising the recombinant expression vector of claim 22.

24. A population of cells comprising at least one host cell of claim 23.

25. A pharmaceutical composition comprising the host cell of claim 23 or a population of cells thereof, and a pharmaceutically acceptable carrier.

26. A method of detecting the presence of cancer in a mammal, comprising:
(a) contacting a sample comprising one or more cells from the mammal with the host cell of claim 23 or a population of cells thereof, thereby forming a complex, and
(b) detecting the complex, wherein detection of the complex is indicative of the presence of cancer in the mammal.

27. A method of treating or preventing cancer in a mammal, the method comprising administering to the mammal an effective amount of the host cell of claim 23.

28. The method of claim 27, wherein the host cell is within a population of cells.

29. The method of claim 27, wherein the host cell is autologous in relation to the mammal.

30. The method of claim 27, wherein the host cell is allogeneic in relation to the mammal.

31. The method of claim 27, wherein the cancer is a hematological malignancy.


SEQUENCE LISTING (50 sequences)

SEQ ID NO: 1 - DNA, 3120 bp, Artificial Sequence
  Synthetic
atggccctgcctgtgacagctctgctgctgcccctggccctgctgctgcatgccgccagacctgagatcgtgctgacccagtctcccggcaccctgtctctcagcccaggagagagagccaccctgagctgcagagccagccagagcgtgtccagcagctacctggcctggtatcagcagaagcccggacaggcccccagactgctgatctacggcgccagctctagagccaccggcatccccgacagattcagcggcagcggcagtggcaccgacttcaccctgaccatcagcagactggaacccgaggacttcgccgtgtactactgccagcagtacggcagcagccggttcaccttcggccctggcaccaaggtggacatcaagggcagcacctccggcagcggcaagcctggctctggcgagggctctaccaagggccaggtgcagctggtgcagtctggcgccgaagtgaagaaacccggctctagcgtgaaggtgtcctgcaaggacagcggcggcaccttcagcagctacgccatcagctgggtgcgccaggccccaggacaggggctggaatggatgggcggcatcatccccatcttcggcaccaccaactacgcccagcagttccagggcagagtgaccatcaccgccgacgagagcaccagcaccgcctacatggaactgagcagcctgcggagcgaggacacagccgtgtattactgtgcccgcgaggccgtggccgccgactggctggatccttggggacagggcaccctggtgacagtgtccagcttcgtgcctgtgtttctgcctgccaagcccaccacaacccctgcccctagacctcctacacccgcccctacaatcgccagccagcctctgtctctgaggcccgaggcttgtagacctgctgctggcggagccgtgcacaccagaggactggatttcgcctgcgacatctacatctgggcccctctggccggcacatgtggcgtgctgctgctgagcctcgtgatcaccctgtactgcaaccaccggaacagaagcaagcggagccggctgctgcacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgatgtgctggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccgggccaagagaagcggcagcggagcccccgtgaagcagaccctgaacttcgacctgctgaaactggccggcgacgtggagagcaaccctggccccatggccctgcctgtgacagctctgctgctgcccctggccctgctgctgcatgccgccagacctgaaatcgtgcttacacaatcacctgcgacactttctctttcccccggtgaaagagcaaccctgtcttgtagagcaagtcaaagcgttagcagttatctcgcatggtaccaacagaagcccggtcaggcgcctcgcctgttgatatacgacgctagtaaccgagcaaccggaatccctgctagattcagtgggtctggaagcggcactgatttcactcttactatctcttctttggagcctgaggactttgcagtttattactgtcagcagagatcagattggcccctcactttcggtggaggcacaaaggttgaaataaaggggggcggaggttcaggcggcggaggaagcggcggggggggctccgaggttcaacttgtccaaagtggagggggactggtccatccgggaggtagcttgcggctctcttgtacaggcagtggattcacgttttcttaccacgcaatgcattgggtgagacaagcacccggtaagggcttggagtgggtatccatcattggcactggcggagtcacctattatgcggacagtgttaagggccgctttactatcagccgcgacaacgttaagaattctctgtatttgcagatgaattcactgagggcggaagacatggccgtttactattgcgccagagactactatggggctggttccttctacgacggtttgtatggtatggatgtctggggccagggcacgacggtaaccgtgtcaagtttcgttccggtttttctgcctgcaaagcctacaactacccccgcaccccggcccccaactcccgctccaacgatcgcatcacaaccactttcactccgaccagaggcttgtagaccggctgcgggaggcgcggtacacacgcgggggctcgattttgcttgcgatatttacatctgggctcctcttgccggcacatgcggtgtcttgctcctgtccctcgtcattactctgtattgcaaccataggaacaagcggggcagaaagaagctgctgtacatcttcaagcagcccttcatgcggcccgtgcagaccacccaggaagaggacggctgctcctgcagattccccgaggaagaagaaggcggctgcgagctgagagtaaaattttccaggtccgcagatgcacccgcttatcagcagggccaaaaccaactgtataatgagttgaacttggggaggcgagaagagtatgacgttttggataaaagacggggacgagaccccgagatgggtggaaagccacggcgcaagaacccgcaagaagggctctataatgaacttcaaaaagacaagatggccgaagcctactcagaaattggcatgaaaggtgagaggaggcgcgggaaaggccatgacgggctttatcaggggttgtcaacggccactaaggatacgtatgacgctctccacatgcaagcgttgcccccccgctaa

SEQ ID NO: 2 - protein, 1039 aa, Artificial Sequence
  Synthetic
MALPVTALLLPLALLLHAARPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRSGSGAPVKQTLNFDLLKLAGDVESNPGPMALPVTALLLPLALLLHAARPEIVLTQSPATLSLSPGERATLSCRASQSVSSYLAWYQQKPGQAPRLLIYDASNRATGIPARFSGSGSGTDFTLTISSLEPEDFAVYYCQQRSDWPLTFGGGTKVEIKGGGGSGGGGSGGGGSEVQLVQSGGGLVHPGGSLRLSCTGSGFTFSYHAMHWVRQAPGKGLEWVSIIGTGGVTYYADSVKGRFTISRDNVKNSLYLQMNSLRAEDMAVYYCARDYYGAGSFYDGLYGMDVWGQGTTVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 21 aa, Homo sapiens
MALPVTALLLPLALLLHAARP

SEQ ID NO: 4 - protein, 108 aa, Artificial Sequence
  Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIK

SEQ ID NO: 5 - protein, 18 aa, Artificial Sequence
  Synthetic
GSTSGSGKPGSGEGSTKG

SEQ ID NO: 6 - protein, 119 aa, Artificial Sequence
  Synthetic
QVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSS

SEQ ID NO: 7 - protein, 83 aa, Homo sapiens
FVPVFLPAKPTTTPAPRPPTPAPTIASQPLSLRPEACRPAAGGAVHTRGLDFACDIYIWAPLAGTCGVLLLSLVITLYCNHRN

SEQ ID NO: 8 - protein, 41 aa, Homo sapiens
RSKRSRLLHSDYMNMTPRRPGPTRKHYQPYAPPRDFAAYRS

SEQ ID NO: 9 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 32 aa, Artificial Sequence
  Sythetic
RAKRSGSGAPVKQTLNFDLLKLAGDVESNPGP

SEQ ID NO: 11 - protein, 107 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSDWPLTFGGGTKVEIK

SEQ ID NO: 12 - protein, 15 aa, Artificial Sequence
  Synthetic
GGGGSGGGGSGGGGS

SEQ ID NO: 13 - protein, 125 aa, Homo sapiens
EVQLVQSGGGLVHPGGSLRLSCTGSGFTFSYHAMHWVRQAPGKGLEWVSIIGTGGVTYYADSVKGRFTISRDNVKNSLYLQMNSLRAEDMAVYYCARDYYGAGSFYDGLYGMDVWGQGTTVTVSS

SEQ ID NO: 14 - protein, 42 aa, Homo sapiens
KRGRKKLLYIFKQPFMRPVQTTQEEDGCSCRFPEEEEGGCEL

SEQ ID NO: 15 - DNA, 3102 bp, Artificial Sequence
  Synthetic
atggccctgcctgtgacagctctgctgctgcccctggccctgctgctgcatgccgccagacctgagatcgtgctgacccagtctcccggcaccctgtctctcagcccaggagagagagccaccctgagctgcagagccagccagagcgtgtccagcagctacctggcctggtatcagcagaagcccggacaggcccccagactgctgatctacggcgccagctctagagccaccggcatccccgacagattcagcggcagcggcagtggcaccgacttcaccctgaccatcagcagactggaacccgaggacttcgccgtgtactactgccagcagtacggcagcagccggttcaccttcggccctggcaccaaggtggacatcaagggcagcacctccggcagcggcaagcctggctctggcgagggctctaccaagggccaggtgcagctggtgcagtctggcgccgaagtgaagaaacccggctctagcgtgaaggtgtcctgcaaggacagcggcggcaccttcagcagctacgccatcagctgggtgcgccaggccccaggacaggggctggaatggatgggcggcatcatccccatcttcggcaccaccaactacgcccagcagttccagggcagagtgaccatcaccgccgacgagagcaccagcaccgcctacatggaactgagcagcctgcggagcgaggacacagccgtgtattactgtgcccgcgaggccgtggccgccgactggctggatccttggggacagggcaccctggtgacagtgtccagcttcgtgcctgtgtttctgcctgccaagcccaccacaacccctgcccctagacctcctacacccgcccctacaatcgccagccagcctctgtctctgaggcccgaggcttgtagacctgctgctggcggagccgtgcacaccagaggactggatttcgcctgcgacatctacatctgggcccctctggccggcacatgtggcgtgctgctgctgagcctcgtgatcaccctgtactgcaaccaccggaacagaagcaagcggagccggctgctgcacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgatgtgctggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccgggccaagagaagcggcagcggagcccccgtgaagcagaccctgaacttcgacctgctgaaactggccggcgacgtggagagcaaccctggccccatggccctgcctgtgacagctctgctgctgcccctggccctgctgctgcatgccgccagacctcagatagttctttcccagtctcctgcaattttgagtgcttccccaggggagaaggtcactatgacctgtagggcaagttcctctgtatcatatattcactggttccagcaaaagcctggttcttcccccaaaccctggatttacgcgactagtaacctggcgtcaggtgtacctgtccggttcagcggaagtggttccgggactagctattctctgactattagcagagtggaggccgaagacgccgcaacctattactgccaacaatggacctcaaatcccccgacatttggcgggggtacaaaactggagatcaaagggggcggaggttcaggcggcggaggaagcggcggggggggctcccaagttcaactgcaacagccgggcgcggagctggtcaagccgggggcttctgtcaagatgagttgtaaggcgtctggctacacattcactagctataatatgcactgggtaaaacaaacgcctggccgcggccttgaatggataggtgccatatatcctggtaatggggatacgtcatacaaccaaaagttcaagggcaaagcgactctcacagcggataagtctagttccaccgcctatatgcagctcagtagtctcacaagtgaagattcagccgtttattattgtgccaggtcaacttactatggaggagattggtacttcaacgtatggggggcgggtactaccgtgaccgtcagcgcattcgttccggtttttctgcctgcaaagcctacaactacccccgcaccccggcccccaactcccgctccaacgatcgcatcacaaccactttcactccgaccagaggcttgtagaccggctgcgggaggcgcggtacacacgcgggggctcgattttgcttgcgatatttacatctgggctcctcttgccggcacatgcggtgtcttgctcctgtccctcgtcattactctgtattgcaaccataggaacaagcggggcagaaagaagctgctgtacatcttcaagcagcccttcatgcggcccgtgcagaccacccaggaagaggacggctgctcctgcagattccccgaggaagaagaaggcggctgcgagctgagagtaaaattttccaggtccgcagatgcacccgcttatcagcagggccaaaaccaactgtataatgagttgaacttggggaggcgagaagagtatgacgttttggataaaagacggggacgagaccccgagatgggtggaaagccacggcgcaagaacccgcaagaagggctctataatgaacttcaaaaagacaagatggccgaagcctactcagaaattggcatgaaaggtgagaggaggcgcgggaaaggccatgacgggctttatcaggggttgtcaacggccactaaggatacgtatgacgctctccacatgcaagcgttgcccccccgc

SEQ ID NO: 16 - protein, 1034 aa, Artificial Sequence
  Synthetic
MALPVTALLLPLALLLHAARPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRSGSGAPVKQTLNFDLLKLAGDVESNPGPMALPVTALLLPLALLLHAARPQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKGGGGSGGGGSGGGGSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 106 aa, Mus musculus [sequence omitted]

SEQ ID NO: 18 - protein, 121 aa, Mus musculus [sequence omitted]

SEQ ID NO: 19 - DNA, 3123 bp, Artificial Sequence
  Synthetic
atggccctgcctgtgacagctctgctgctgcccctggccctgctgctgcatgccgccagacctgagatcgtgctgacccagtctcccggcaccctgtctctcagcccaggagagagagccaccctgagctgcagagccagccagagcgtgtccagcagctacctggcctggtatcagcagaagcccggacaggcccccagactgctgatctacggcgccagctctagagccaccggcatccccgacagattcagcggcagcggcagtggcaccgacttcaccctgaccatcagcagactggaacccgaggacttcgccgtgtactactgccagcagtacggcagcagccggttcaccttcggccctggcaccaaggtggacatcaagggcagcacctccggcagcggcaagcctggctctggcgagggctctaccaagggccaggtgcagctggtgcagtctggcgccgaagtgaagaaacccggctctagcgtgaaggtgtcctgcaaggacagcggcggcaccttcagcagctacgccatcagctgggtgcgccaggccccaggacaggggctggaatggatgggcggcatcatccccatcttcggcaccaccaactacgcccagcagttccagggcagagtgaccatcaccgccgacgagagcaccagcaccgcctacatggaactgagcagcctgcggagcgaggacacagccgtgtattactgtgcccgcgaggccgtggccgccgactggctggatccttggggacagggcaccctggtgacagtgtccagcttcgtgcctgtgtttctgcctgccaagcccaccacaacccctgcccctagacctcctacacccgcccctacaatcgccagccagcctctgtctctgaggcccgaggcttgtagacctgctgctggcggagccgtgcacaccagaggactggatttcgcctgcgacatctacatctgggcccctctggccggcacatgtggcgtgctgctgctgagcctggtcatcaccctgtactgcaaccaccggaacagaagcaagcggagcagactgctgcacagcgactacatgaacatgacccctagacggcccggacctaccagaaagcactaccagccttacgctcctcctcgggactttgccgcctatcggagcagagtgaagttcagcagatcagccgatgctcctgcctaccagcagggccagaatcagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggcatgaagggcgagagaagaagaggcaagggccacgatggactgtaccagggactgagcacagccaccaaggatacctacgatgccctgcacatgcaggcccttccacctagaagggccaagagatctggatctggcgcccctgtgaagcagaccctgaatttcgacctgctgaagctggccggcgacgtggaatctaatcctggacctatggctctgcccgtgacagctttgctgctgcctctggctctgctgctgcatgccgctagacccgatatcgtgatgacccagacacctcacagcagccctgttacactgggacagcctgccagcatctcctgtagaagcagccagagcctggtgtccagagatggcaatacctacctgagctggctgcagcagaggcctggacaacctcctagactgctgatctacaagatcagcaaccggttcagcggcgtgcccaatagattttctggaagcggagccggcaccgacttcaccctgaagatttctagagtgaaggccgaggacgtgggcgtgtactactgtatgcaggccacacagttccctctgacctttggccagggcaccagactggaaatcaaaggtggcggaggttctggcggcggaggatcaggcggaggtggaagtgaagtgcagctggttcagtctggcgccgaagtgaagaagcctggcgagtctctgaagatcagctgcaaaggcagcggctacagcttcaccagctattggatcggctgggtccgacagatgcctggcaaaggactggaatggatgggcatcatctaccccggcgacagcgataccagatacagccctagctttcagggccaagtgaccatcagcgccgacaagagcatcagcacagcctacctgcagtggtctagcctgaaggccagcgacaccgccatgtactattgtgccagacagggcgacttttggagcggctatggtggcatggatgtgtggggacagggcacaacagtgaccgtgtctagcttcgtgcctgtgttcctgcctgccaagcctacaacaacccctgctcctagacctcctacaccagctcctacaatcgccagccagcctctgtctctgaggcctgaagcttgtagacctgctgctggcggagccgtgcataccagaggactggatttcgcctgcgacatctacatttgggcccctctggctggaacttgtggcgtgctgctgctgtctctcgtgatcacactgtattgcaatcataggaacaagcgaggccggaagaagctgctgtacatcttcaagcagcctttcatgcggcccgtgcagaccacacaagaggaagatggctgtagctgcagattccccgaggaagaagaaggcggctgcgagctgagagtgaaattctctagaagcgccgacgcacccgcataccagcaaggacaaaaccagctctataacgaactcaacctcggcagacgcgaggaatatgatgtgctggacaagaggcggggacgcgatccagaaatgggaggaaagcctcggagaaagaacccacaagagggactttacaacgaactccaaaaggataagatggcagaagcctattccgagattggaatgaagggcgaacgtcggagaggaaagggacacgacggcctttatcagggcctgtccaccgccacaaaagatacgtatgacgctctccacatgcaagcgttgcccccccgc

SEQ ID NO: 20 - protein, 1041 aa, Artificial Sequence
  Synthetic
MALPVTALLLPLALLLHAARPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRSGSGAPVKQTLNFDLLKLAGDVESNPGPMALPVTALLLPLALLLHAARPDIVMTQTPHSSPVTLGQPASISCRSSQSLVSRDGNTYLSWLQQRPGQPPRLLIYKISNRFSGVPNRFSGSGAGTDFTLKISRVKAEDVGVYYCMQATQFPLTFGQGTRLEIKGGGGSGGGGSGGGGSEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQGDFWSGYGGMDVWGQGTTVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 112 aa, Homo sapiens
DIVMTQTPHSSPVTLGQPASISCRSSQSLVSRDGNTYLSWLQQRPGQPPRLLIYKISNRFSGVPNRFSGSGAGTDFTLKISRVKAEDVGVYYCMQATQFPLTFGQGTRLEIK

SEQ ID NO: 22 - protein, 122 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQGDFWSGYGGMDVWGQGTTVTVSS

SEQ ID NO: 23 - DNA, 3111 bp, Artificial Sequence
  Synthetic
atggccctgcctgtgacagctctgctgctgcccctggccctgctgctgcatgccgccagacctgagatcgtgctgacccagtctcccggcaccctgtctctcagcccaggagagagagccaccctgagctgcagagccagccagagcgtgtccagcagctacctggcctggtatcagcagaagcccggacaggcccccagactgctgatctacggcgccagctctagagccaccggcatccccgacagattcagcggcagcggcagtggcaccgacttcaccctgaccatcagcagactggaacccgaggacttcgccgtgtactactgccagcagtacggcagcagccggttcaccttcggccctggcaccaaggtggacatcaagggcagcacctccggcagcggcaagcctggctctggcgagggctctaccaagggccaggtgcagctggtgcagtctggcgccgaagtgaagaaacccggctctagcgtgaaggtgtcctgcaaggacagcggcggcaccttcagcagctacgccatcagctgggtgcgccaggccccaggacaggggctggaatggatgggcggcatcatccccatcttcggcaccaccaactacgcccagcagttccagggcagagtgaccatcaccgccgacgagagcaccagcaccgcctacatggaactgagcagcctgcggagcgaggacacagccgtgtattactgtgcccgcgaggccgtggccgccgactggctggatccttggggacagggcaccctggtgacagtgtccagcttcgtgcctgtgtttctgcctgccaagcccaccacaacccctgcccctagacctcctacacccgcccctacaatcgccagccagcctctgtctctgaggcccgaggcttgtagacctgctgctggcggagccgtgcacaccagaggactggatttcgcctgcgacatctacatctgggcccctctggccggcacatgtggcgtgctgctgctgagcctggtcatcaccctgtactgcaaccaccggaacagaagcaagcggagcagactgctgcacagcgactacatgaacatgacccctagacggcccggacctaccagaaagcactaccagccttacgctcctcctagagacttcgccgcctaccggtccagagtgaagttcagcagatccgccgatgctcccgcctatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaagggccaagagatctggatctggcgcccctgtgaagcagaccctgaacttcgacctgctgaaactggccggcgacgtggaaagcaaccctggacctatggctctgcctgtgacagctctgctgctgcctctggctctgcttctgcatgccgccagacctgagatcgtgatgacacagtctcccgccacactgagcatgagccctggcgaaagagccacactgtcctgtagagccagccagagcgtgtccagaaacctggcctggtatcagcagaaagtcggacaggcccctcggctgcttatctctggcgctagcacaagagccaccggcattccagccagattttctggcagcggctccggcaccgagttcaccctgacaatcaatagcctgcagagcgaggatttcgccgtgtactactgccagcagagcaacgactggcctctgacctttggccagggcaccagactggaaatcaaaggcggcggaggaagcggaggcggaggttctggtggcggaggatctgaagtgcagctggctgaatcaggcggcgatctggtgcagtctggcagaagcctgagactgtcttgtgccgccagcggcatcaccttccacgattatgccatgcactgggtccgacagcctccaggcaaaggccttgaatgggtgtccggcatcagctggaactccgactacatcggctacgccgacagcgtgaagggcagattcaccatctccagagacaacgccaagaagtccctgtacctgcagatgaacagcctgcggcctgacgacacagccctgtactattgcgtgaaggacttccactacggcagcggcagcaactacggcatggatgtttggggccagggaaccaccgtgaccgtgtctagtttcgtgcccgtgttcctgcctgccaagcctacaacaacccctgctcctagacctcctacaccagctcctacaatcgccagccagcctctgtctctgaggccagaggcttgtagacctgctgctggcggagccgtgcatacaagaggactggacttcgcctgcgacatctacatctgggctcctctggccggaacatgtggcgtgctgttgctgtctctcgtgatcacactgtattgcaatcataggaacaagcgaggccggaagaagctgctgtacatcttcaagcagcccttcatgcggcccgtgcagaccacacaagaggaagatggctgctcctgcagattccccgaggaagaagaaggcggctgcgagctgcgcgtgaagttttctagaagcgctgacgcccctgcttaccagcagggccaaaatcagctctataacgaactgaatctcggcaggcgcgaggaatatgatgtgctggataagaggcgcggcagggacccagagatgggaggaaagcctcggagaaagaacccacaagaaggcctttacaacgaactgcaaaaggataagatggctgaagcctattccgagattggcatgaagggcgaacgtcggagaggaaaaggccacgacggactctatcagggactgtctacagccacaaaagatacgtatgacgctctccacatgcaagcgttgcccccccgc

SEQ ID NO: 24 - protein, 1037 aa, Artificial Sequence
  Synthetic
MALPVTALLLPLALLLHAARPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRSGSGAPVKQTLNFDLLKLAGDVESNPGPMALPVTALLLPLALLLHAARPEIVMTQSPATLSMSPGERATLSCRASQSVSRNLAWYQQKVGQAPRLLISGASTRATGIPARFSGSGSGTEFTLTINSLQSEDFAVYYCQQSNDWPLTFGQGTRLEIKGGGGSGGGGSGGGGSEVQLAESGGDLVQSGRSLRLSCAASGITFHDYAMHWVRQPPGKGLEWVSGISWNSDYIGYADSVKGRFTISRDNAKKSLYLQMNSLRPDDTALYYCVKDFHYGSGSNYGMDVWGQGTTVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 107 aa, Homo sapiens
EIVMTQSPATLSMSPGERATLSCRASQSVSRNLAWYQQKVGQAPRLLISGASTRATGIPARFSGSGSGTEFTLTINSLQSEDFAVYYCQQSNDWPLTFGQGTRLEIK

SEQ ID NO: 26 - protein, 123 aa, Homo sapiens
EVQLAESGGDLVQSGRSLRLSCAASGITFHDYAMHWVRQPPGKGLEWVSGISWNSDYIGYADSVKGRFTISRDNAKKSLYLQMNSLRPDDTALYYCVKDFHYGSGSNYGMDVWGQGTTVTVSS

SEQ ID NO: 27 - DNA, 3114 bp, Artificial Sequence
  Synthetic
atggccctgcctgtgacagctctgctgctgcccctggccctgctgctgcatgccgccagacctgagatcgtgctgacccagtctcccggcaccctgtctctcagcccaggagagagagccaccctgagctgcagagccagccagagcgtgtccagcagctacctggcctggtatcagcagaagcccggacaggcccccagactgctgatctacggcgccagctctagagccaccggcatccccgacagattcagcggcagcggcagtggcaccgacttcaccctgaccatcagcagactggaacccgaggacttcgccgtgtactactgccagcagtacggcagcagccggttcaccttcggccctggcaccaaggtggacatcaagggcagcacctccggcagcggcaagcctggctctggcgagggctctaccaagggccaggtgcagctggtgcagtctggcgccgaagtgaagaaacccggctctagcgtgaaggtgtcctgcaaggacagcggcggcaccttcagcagctacgccatcagctgggtgcgccaggccccaggacaggggctggaatggatgggcggcatcatccccatcttcggcaccaccaactacgcccagcagttccagggcagagtgaccatcaccgccgacgagagcaccagcaccgcctacatggaactgagcagcctgcggagcgaggacacagccgtgtattactgtgcccgcgaggccgtggccgccgactggctggatccttggggacagggcaccctggtgacagtgtccagcttcgtgcctgtgtttctgcctgccaagcccaccacaacccctgcccctagacctcctacacccgcccctacaatcgccagccagcctctgtctctgaggcccgaggcttgtagacctgctgctggcggagccgtgcacaccagaggactggatttcgcctgcgacatctacatctgggcccctctggccggcacatgtggcgtgctgctgctgagcctcgtgatcaccctgtactgcaaccaccggaacagaagcaagcggagccggctgctgcacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgatgtgctggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccgggccaagagaagcggcagcggagcccccgtgaagcagaccctgaacttcgacctgctgaaactggccggcgacgtggagagcaaccctggccccatggccctgcctgtgacagctctgctgctgcccctggccctgctgctgcatgccgccagacctgacatagtaatgacacaaactcctttgtctttgccagttactccgggggaaccagccagcatcagttgtcggtctagtaagtcactgttgcattccaacggtataacctatctttactggtatttgcagaaaccgggtcaatccccccagttgctcatttaccagatgagtaacctcgttagcggtgtccccgacaggttctcagggtcaggtagtgggacggatttcacgctgaaaatttccagagttgaggccgaggatgttggagtgtactattgtgcacagaatctcgaattgccatacacgtttggaggcggtacgaaagtagagataaaagggggcggaggttcaggcggcggaggaagcggcggggggggctcccaggtacaactcgtacaaagtggcgccgaagtcaagaaacctggctcctctgtaaaggtctcatgtaaagcatcagggtacgctttttcatacagttggatcaattgggtacgacaggctcctggccagggcttggagtggatgggccgcatctttccaggtgatggcgacacggattacaatgggaagttcaaagggcgagtaactatcacagcagataaaagcacttccacagcgtacatggaactctcctcactgaggtctgaggatacagcggtgtattattgtgcgcgaaatgtgttcgacggttattggttggtgtattggggacaaggcaccctcgttacggtgagtagcttcgttccggtttttctgcctgcaaagcctacaactacccccgcaccccggcccccaactcccgctccaacgatcgcatcacaaccactttcactccgaccagaggcttgtagaccggctgcgggaggcgcggtacacacgcgggggctcgattttgcttgcgatatttacatctgggctcctcttgccggcacatgcggtgtcttgctcctgtccctcgtcattactctgtattgcaaccataggaacaagcggggcagaaagaagctgctgtacatcttcaagcagcccttcatgcggcccgtgcagaccacccaggaagaggacggctgctcctgcagattccccgaggaagaagaaggcggctgcgagctgagagtaaaattttccaggtccgcagatgcacccgcttatcagcagggccaaaaccaactgtataatgagttgaacttggggaggcgagaagagtatgacgttttggataaaagacggggacgagaccccgagatgggtggaaagccacggcgcaagaacccgcaagaagggctctataatgaacttcaaaaagacaagatggccgaagcctactcagaaattggcatgaaaggtgagaggaggcgcgggaaaggccatgacgggctttatcaggggttgtcaacggccactaaggatacgtatgacgctctccacatgcaagcgttgcccccccgc

SEQ ID NO: 28 - protein, 1038 aa, Artificial Sequence
  Synthetic
MALPVTALLLPLALLLHAARPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRSGSGAPVKQTLNFDLLKLAGDVESNPGPMALPVTALLLPLALLLHAARPDIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKGGGGSGGGGSGGGGSQVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 112 aa, Artificial Sequence
  Synthetic
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 30 - protein, 119 aa, Artificial Sequence
  Synthetic
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 31 - DNA, 1521 bp, Artificial Sequence
  Synthetic
atggctctgcccgtgacagctttgctgctgcctctggctctgctgctgcatgccgctagacccgatatcgtgatgacccagacacctcacagcagccctgttacactgggacagcctgccagcatctcctgtagaagcagccagagcctggtgtccagagatggcaatacctacctgagctggctgcagcagaggcctggacaacctcctagactgctgatctacaagatcagcaaccggttcagcggcgtgcccaatagattttctggaagcggagccggcaccgacttcaccctgaagatttctagagtgaaggccgaggacgtgggcgtgtactactgtatgcaggccacacagttccctctgacctttggccagggcaccagactggaaatcaaaggtggcggaggttctggcggcggaggatcaggcggaggtggaagtgaagtgcagctggttcagtctggcgccgaagtgaagaagcctggcgagtctctgaagatcagctgcaaaggcagcggctacagcttcaccagctattggatcggctgggtccgacagatgcctggcaaaggactggaatggatgggcatcatctaccccggcgacagcgataccagatacagccctagctttcagggccaagtgaccatcagcgccgacaagagcatcagcacagcctacctgcagtggtctagcctgaaggccagcgacaccgccatgtactattgtgccagacagggcgacttttggagcggctatggtggcatggatgtgtggggacagggcacaacagtgaccgtgtctagcttcgtgcctgtgttcctgcctgccaagcctacaacaacccctgctcctagacctcctacaccagctcctacaatcgccagccagcctctgtctctgaggcctgaagcttgtagacctgctgctggcggagccgtgcataccagaggactggatttcgcctgcgacatctacatttgggcccctctggctggaacttgtggcgtgctgctgctgtctctcgtgatcacactgtattgcaatcataggaacaagcgaggccggaagaagctgctgtacatcttcaagcagcctttcatgcggcccgtgcagaccacacaagaggaagatggctgtagctgcagattccccgaggaagaagaaggcggctgcgagctgagagtgaaattctctagaagcgccgacgcacccgcataccagcaaggacaaaaccagctctataacgaactcaacctcggcagacgcgaggaatatgatgtgctggacaagaggcggggacgcgatccagaaatgggaggaaagcctcggagaaagaacccacaagagggactttacaacgaactccaaaaggataagatggcagaagcctattccgagattggaatgaagggcgaacgtcggagaggaaagggacacgacggcctttatcagggcctgtccaccgccacaaaagatacgtatgacgctctccacatgcaagcgttgcccccccgc

SEQ ID NO: 32 - protein, 507 aa, Artificial Sequence
  Synthetic
MALPVTALLLPLALLLHAARPDIVMTQTPHSSPVTLGQPASISCRSSQSLVSRDGNTYLSWLQQRPGQPPRLLIYKISNRFSGVPNRFSGSGAGTDFTLKISRVKAEDVGVYYCMQATQFPLTFGQGTRLEIKGGGGSGGGGSGGGGSEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQGDFWSGYGGMDVWGQGTTVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - DNA, 1509 bp, Artificial Sequence
  Synthetic
atggctctgcctgtgacagctctgctgctgcctctggctctgcttctgcatgccgccagacctgagatcgtgatgacacagtctcccgccacactgagcatgagccctggcgaaagagccacactgtcctgtagagccagccagagcgtgtccagaaacctggcctggtatcagcagaaagtcggacaggcccctcggctgcttatctctggcgctagcacaagagccaccggcattccagccagattttctggcagcggctccggcaccgagttcaccctgacaatcaatagcctgcagagcgaggatttcgccgtgtactactgccagcagagcaacgactggcctctgacctttggccagggcaccagactggaaatcaaaggcggcggaggaagcggaggcggaggttctggtggcggaggatctgaagtgcagctggctgaatcaggcggcgatctggtgcagtctggcagaagcctgagactgtcttgtgccgccagcggcatcaccttccacgattatgccatgcactgggtccgacagcctccaggcaaaggccttgaatgggtgtccggcatcagctggaactccgactacatcggctacgccgacagcgtgaagggcagattcaccatctccagagacaacgccaagaagtccctgtacctgcagatgaacagcctgcggcctgacgacacagccctgtactattgcgtgaaggacttccactacggcagcggcagcaactacggcatggatgtttggggccagggaaccaccgtgaccgtgtctagtttcgtgcccgtgttcctgcctgccaagcctacaacaacccctgctcctagacctcctacaccagctcctacaatcgccagccagcctctgtctctgaggccagaggcttgtagacctgctgctggcggagccgtgcatacaagaggactggacttcgcctgcgacatctacatctgggctcctctggccggaacatgtggcgtgctgttgctgtctctcgtgatcacactgtattgcaatcataggaacaagcgaggccggaagaagctgctgtacatcttcaagcagcccttcatgcggcccgtgcagaccacacaagaggaagatggctgctcctgcagattccccgaggaagaagaaggcggctgcgagctgcgcgtgaagttttctagaagcgctgacgcccctgcttaccagcagggccaaaatcagctctataacgaactgaatctcggcaggcgcgaggaatatgatgtgctggataagaggcgcggcagggacccagagatgggaggaaagcctcggagaaagaacccacaagaaggcctttacaacgaactgcaaaaggataagatggctgaagcctattccgagattggcatgaagggcgaacgtcggagaggaaaaggccacgacggactctatcagggactgtctacagccacaaaagatacgtatgacgctctccacatgcaagcgttgcccccccgc

SEQ ID NO: 34 - protein, 503 aa, Artificial Sequence
  Synthetic
MALPVTALLLPLALLLHAARPEIVMTQSPATLSMSPGERATLSCRASQSVSRNLAWYQQKVGQAPRLLISGASTRATGIPARFSGSGSGTEFTLTINSLQSEDFAVYYCQQSNDWPLTFGQGTRLEIKGGGGSGGGGSGGGGSEVQLAESGGDLVQSGRSLRLSCAASGITFHDYAMHWVRQPPGKGLEWVSGISWNSDYIGYADSVKGRFTISRDNAKKSLYLQMNSLRPDDTALYYCVKDFHYGSGSNYGMDVWGQGTTVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - DNA, 1500 bp, Artificial Sequence
  Synthetic
atggccctgcctgtgacagctctgctgctgcccctggccctgctgctgcatgccgccagacctcagatagttctttcccagtctcctgcaattttgagtgcttccccaggggagaaggtcactatgacctgtagggcaagttcctctgtatcatatattcactggttccagcaaaagcctggttcttcccccaaaccctggatttacgcgactagtaacctggcgtcaggtgtacctgtccggttcagcggaagtggttccgggactagctattctctgactattagcagagtggaggccgaagacgccgcaacctattactgccaacaatggacctcaaatcccccgacatttggcgggggtacaaaactggagatcaaagggggcggaggttcaggcggcggaggaagcggcggggggggctcccaagttcaactgcaacagccgggcgcggagctggtcaagccgggggcttctgtcaagatgagttgtaaggcgtctggctacacattcactagctataatatgcactgggtaaaacaaacgcctggccgcggccttgaatggataggtgccatatatcctggtaatggggatacgtcatacaaccaaaagttcaagggcaaagcgactctcacagcggataagtctagttccaccgcctatatgcagctcagtagtctcacaagtgaagattcagccgtttattattgtgccaggtcaacttactatggaggagattggtacttcaacgtatggggggcgggtactaccgtgaccgtcagcgcattcgttccggtttttctgcctgcaaagcctacaactacccccgcaccccggcccccaactcccgctccaacgatcgcatcacaaccactttcactccgaccagaggcttgtagaccggctgcgggaggcgcggtacacacgcgggggctcgattttgcttgcgatatttacatctgggctcctcttgccggcacatgcggtgtcttgctcctgtccctcgtcattactctgtattgcaaccataggaacaagcggggcagaaagaagctgctgtacatcttcaagcagcccttcatgcggcccgtgcagaccacccaggaagaggacggctgctcctgcagattccccgaggaagaagaaggcggctgcgagctgagagtaaaattttccaggtccgcagatgcacccgcttatcagcagggccaaaaccaactgtataatgagttgaacttggggaggcgagaagagtatgacgttttggataaaagacggggacgagaccccgagatgggtggaaagccacggcgcaagaacccgcaagaagggctctataatgaacttcaaaaagacaagatggccgaagcctactcagaaattggcatgaaaggtgagaggaggcgcgggaaaggccatgacgggctttatcaggggttgtcaacggccactaaggatacgtatgacgctctccacatgcaagcgttgcccccccgc

SEQ ID NO: 36 - protein, 500 aa, Artificial Sequence
  Synthetic
MALPVTALLLPLALLLHAARPQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKGGGGSGGGGSGGGGSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 11 aa, Homo sapiens
RASQSVSSYLA

SEQ ID NO: 38 - protein, 7 aa, Homo sapiens
DASNRAT

SEQ ID NO: 39 - protein, 9 aa, Homo sapiens
QQRSDWPLT

SEQ ID NO: 40 - protein, 6 aa, Homo sapiens
SYHAMH

SEQ ID NO: 41 - protein, 16 aa, Homo sapiens
IIGTGGVTYYADSVKG

SEQ ID NO: 42 - protein, 17 aa, Homo sapiens
DYYGAGSFYDGLYGMDV

SEQ ID NO: 43 - protein, 16 aa, Artificial Sequence
  Synthetic
RSSKSLLHSNGITYLY

SEQ ID NO: 44 - protein, 7 aa, Artificial Sequence
  Synthetic
QMSNLVS

SEQ ID NO: 45 - protein, 9 aa, Artificial Sequence
  Synthetic
AQNLELPYT

SEQ ID NO: 46 - protein, 6 aa, Artificial Sequence
  Synthetic
GYAFSY

SEQ ID NO: 47 - protein, 8 aa, Artificial Sequence
  Synthetic
FPGDGDTD

SEQ ID NO: 48 - protein, 10 aa, Artificial Sequence
  Synthetic
NVFDGYWLVY

SEQ ID NO: 49 - protein, 9 aa, Artificial Sequence
  Synthetic
  misc_feature (5)..(5): Xaa can be any naturally occurring amino acid
GDVEXNPGP

SEQ ID NO: 50 - protein, 4 aa, Artificial Sequence
  Synthetic
SGSG